(19) United States
(12) Patent Application Publication
(10) Pub. No.: US 2003/0059827 A1
(43) Pub. Date: Mar. 27, 2003

(54) Engineered protein binding domains and methods and systems for their design and use

(76) Inventors: Cayetano Gonzalez, Heidelberg (DE); Emmanuel Lacroix, Paris (FR); Jose Reina, Heidelberg (DE); Luis Serrano, Heidelberg (DE)

Correspondence Address: PENNIE AND EDMONDS, 1155 AVENUE OF THE AMERICAS, NEW YORK, NY, 100362711

(21) Appl. No.: 09/805,353

(22) Filed: Mar. 13, 2001

(51) Int. Cl. C12Q 1/68; G01N 33/53; G06F 19/00

(52) U.S. Cl. 435/7.1; 435/6; 702/19

(57) ABSTRACT

The present invention rapidly and efficiently provides proteins engineered to bind to arbitrary target proteins requiring only knowledge of the amino acid sequences of short portions of the target proteins (for example, either the amino or the carboxy termini). This invention provides such proteins as well as methods and systems for their design, synthesis and use, and especially provides for use of a plurality of binding proteins in array format. The engineering methods of the present invention take a precursor protein known to already bind to a short peptide and engineer alterations in precursor proteins so that it binds to a new target peptide by using computer-assisted molecular design techniques and optional assay for actual binding. The invention also provides arrays and libraries of binding proteins and methods of using binding proteins.

### FIELD OF THE INVENTION

The present invention relates to biotechnology, specifically to protein technology, and provides binding polypeptides engineered from precursor polypeptides that bind to selected, preferably short, target polypeptides. Characteristically, only the primary amino acid sequences of the short targets, for example, sequences of their N- or C-termini, are used for this engineering; knowledge of the target's spatial structures is not needed or used. This invention also provides methods and systems for this polypeptides engineering, as well as devices and methods for use of the polypeptides, especially as spatial arrays or recombinant libraries.

### BACKGROUND OF THE INVENTION

Availability of naturally-occurring proteins (or non-natural polypeptides in general) that specifically bind or interact with target proteins or molecules has for some time been of importance in biology and medicine. For example, medical diagnosis has been revolutionized by assays using high-affinity proteins, mainly antibodies, that bind to disease markers. High-affinity antibodies to disease-causing agents are of increasing importance in medical therapeutics. In biological research, high affinity proteins, also mainly antibodies, have found use in the purification of rare proteins, in the localization of proteins or other antigens in cells such as by immuno-histochemical techniques, and in countless other applications. High-affinity proteins are likely to assume increasing research importance in the future. For example, the emerging field of proteomics seeks to understand the patterns of expression and interaction of a substantial fraction of the proteins encoded in a cell's genome. However, progress in proteomics will be severely hindered without economical and efficient assays for substantial fractions of cellular proteins. Polypeptides that specifically bind to large numbers of individual cellular proteins will make such economical proteomic assays possible, as have oligonucleotides that bind to large numbers of cellular nucleic acids (for example, in array formats) have done for genomics.

However, with existing methods, providing binding proteins or polypeptides that bind with affinity and specificity to selected targets, especially to large number of selected targets, has been and continues to be too difficult and too expensive.

One well-known existing method is to raise antibodies, either monoclonal or polyclonal, against a target molecule. Although well known, this strategy has several limitations and disadvantages. First, to raise an antibody requires in the first place either a sufficient amount of the purified target or a chemically synthesized target having an epitope known to be shared with the desired target. Second, raising an antibody normally requires use of living animals, and due to species incompatibilities, it is not always possible to raise a specific antibody against a particular target, much less against large numbers of targets, such as a significant fraction of the proteins in an organism. Third, production of antibodies is expensive and time consuming (usually requiring at least two months). Finally, antibodies cannot be routinely expressed in vivo, because the antigen-binding regions of the antibody heavy and light chains must be sequenced, and then the binding regions of both chains must then be simultaneously expressed. Even if expression is achieved, antibodies normally do not fold properly in the reductive cell environment to result in a functional intracellular antibody. Therefore, they are usually micro-injected if needed intracellularly.

Instead of raising antibodies to the target itself, a further known method for obtaining binding proteins is known as epitope tagging. See, e.g., Jarvik et al., 1988, Ann. Rev. Genet. 32:601-18. In this strategy, antibodies are raised against an epitope defined by small, known peptide sequence. Then using known recombinant techniques, which do not necessarily require isolation of the target protein, the gene for the target protein is modified by attaching a DNA sequence for the small target protein to the N- or C-terminus of the target, so that the expressed protein is a fusion of the target with the epitope recognized by the antibodies. Alternatively, if the small peptide is a nine amino acid peptide that binds specifically to streptavidin in the pocket normally occupied by biotin, then a resulting fusion protein may be a target for streptavidin. See, e.g., Skerra et al., 1999, Biomol. Eng. 16:79-86. Further similar known methods are described in Rigaut et al., 1999, Nat. Biotech. 17:1030-32. Among the disadvantages of these strategies are that the target protein needs to be modified, thus requiring additional time and expense, and possibly impairing its normal properties, roles, and expression levels in the cell.

Another known method relies on recombinant techniques to alter the binding specificity of known naturally-occurring proteins that are known to bind to determined targets. In this method, a known protein is randomly mutated by a chemical or biotechnological mutagenesis techniques, for example, by PCR-based mutagenesis. Then a library of the resulting mutated proteins is screened for affinity to a new target, for example, by a yeast two-hybrid assay. See, e.g., Schneider et al., 1999, Nature Biotechnology 17:170-175 (altered binding specificity of a naturally-occurring PDZ domain). Instead of a two-hybrid method, phage display may be used as to assay the library produced by random mutagenesis. See, e.g., Fuli et al., 2000, J. Biol. Chem. 275:21486-91. The experimental work required, random mutagenesis and library screening, for this method to find even a single binding polypeptide for a new target is also often tedious and costly.

Alternatively, binding protein design may be attempted by computational methods, for example, by known computational methods for ab initio protein structure prediction. These computational methods seem to offer several apparent benefits, such as reduced cost and time by avoiding experimental effort, scalability for determining binding proteins to multiple targets, and a broad range of applications. On the other hand these methods have certain notable drawbacks, the principal of which being the well-known extreme difficulty of computing protein structures ab initio. Therefore, computational methods often begin with known three-dimensional structures for the binding protein, the bound target protein, and the unbound target protein. However, determination of these three-dimensional protein structures, using either X-ray diffraction or nuclear magnetic resonance techniques, remains quite time-consuming, costly, and even not always possible.

In summary, polypeptides capable of binding to specific targets, especially to naturally peptide sequences, are useful in biology and medicine, and are expected to be of increasing usefulness in the future. But the current art offers no methods sufficiently efficient and economical to meet demands for large numbers of binding proteins. Existing methods are time consuming, often costly, and may have additional drawbacks. Therefore, cheap and efficient methods for providing plentiful binding proteins to large numbers of arbitrarily selected target proteins are needed.

Citation or identification of any reference in this Section (or in any section of this application) shall not be construed that such reference is available as prior art to the present invention.

### SUMMARY OF THE INVENTION

The present invention has for its objects, and solves the problems of, rapidly and economically providing macromolecules that are capable of specifically binding with selected target molecules, and that may be used as a replacement for current binding macromolecules. These objects are achieved, according to the present invention, by systematically modifying, or engineering according to rational methods, a precursor macromolecule that initially binds with a known target so that it binds with a new, selected target instead of its initial, known target. In its most general embodiments, the present invention applies to “macromolecules”, which refer herein to polymeric molecules (also called “polymers”), either naturally occurring or artificially synthesized and which consist of a sequence of identifiable, structurally-similar subunits (also called “building blocks” or “monomers”; bound forms of building blocks often being referred to differently, for example, the bound form of an amino acids being termed residues).

In preferred embodiments, the macromolecules and their building blocks are naturally occurring monomers, such as naturally occurring amino acids, nucleosides, carbohydrates (saccharides), lipids, and so forth, but the invention is not so limited. Macromolecules may include non-naturally occurring monomers. Therefore, macromolecules engineered according to the invention may be long and short linear polypeptides, cyclic polypeptides, polynucleotides, lipids, polysaccharides, and so forth. In the more preferred embodiments, the present invention applies to polypeptide macromolecules, preferably to polymers of the naturally occurring amino acids. Such polypeptides, especially naturally occurring polypeptides, are called “proteins” herein. However, this invention also includes polypeptides with non-natural, or artificial, amino acids, e.g., d-amino acids. In theses embodiments, the target macromolecules are also polypeptides, and more preferably proteins (naturally occurring polypeptides).

Applications of these more preferred embodiments are based on innovative combinations of certain observations characterizing proteins together with iterative use of rational, especially computer based and precursor-based, polypeptide design methods. First, the binding polypeptides are advantageously targeted to bind with peptides or with peptide sub-sequences of larger polypeptides. The term “peptide” is generally taken herein to refer to shorter polypeptides, for example, shorter than about 50, or 25, or 10, or 5 residues, and “peptide sequences” are sub-sequences of larger polypeptides of these short lengths. It has been observed that natural proteins, especially the proteins present in a single organism, have a very limited set of amino acid sequences. Indeed, most proteins can be uniquely distinguished by short peptide sub-sequences. Terminal peptide sequences, either C- or N-terminal, of 5-10 amino acid residues can uniquely identify most proteins present even in humans. Therefore, by targeting polypeptides to bind peptide sequences the present invention provides binding partners for virtually all (accessible) proteins in an organism.

Further, the present invention achieves significant efficiencies in providing polypeptides targeted to bind to peptide sequences by employing rational, especially computer based and precursor-based, polypeptide design methods. These methods first start with a precursor which already binds a target similar to the targeted peptide sequence. For example, if the targeted sequence is at a protein N-terminal, then the precursor preferably binds the N-terminal of a peptide of similar length. (Similarly, for macromolecules, targeting preferably starts with a macromolecule already binding a similar target macromolecule.) Next, the precursor is redesigned by rational methods so that it binds to the new target. Because this redesign preferably chooses new amino acids in the precursor's typically limited binding domain that interact specifically with the new target, most of the precursor's spatial structure may be assumed substantially fixed. The precursor spatial structure may therefore be obtained from protein structure databases, and since short peptides have little or no fixed structure, the rational methods employed in this invention avoid many difficult problems of protein structure determination. Thereby, they are more efficient.

This invention further preferably employs rational methods in an iterative fashion, using fast approximate to select and screen numerous candidates, before using more accurate but slower methods. After one or more “select and test” iterations, the redesigned candidate polypeptides are preferably focused, for example, by being limited to no more than 10,000, or preferably to no more than 1,000 (more preferably, to 100 or less) candidates. Preferably, but not in all cases, these focused candidates can then be screened by laboratory methods for actual binding to the new target peptide sequence or peptides. In laboratory screening, site-directed mutagenesis centered at the target binding region or pocket (defined subsequently) and limited to a few optimum amino acids mutations is preferably used to construct limited libraries for such actual screening.

Many rational protein design methods may be employed in this invention. For example, they may include application of heuristics or empirical rules for predicting protein structure known in the art, or structure approximation by homology to known structures, or use of known computational methods for ab initio predicting protein structure. These rational, computational methods offer several benefits, such as reduced cost and time, scalability for determining binding proteins to multiple targets, ease of automation, and a broad range of applications. The invention preferably applies, during one or more “select and test” iterations, computer-assisted molecular design (CAMD) methods that utilize approximations appropriate to the constraints of this application, for example, approximations such as the inverse folding approach in combination with rotamer libraries. Appropriate approximation achieve accuracy along with quite significant efficiencies with respect to more ab initio methods and with respect to in vitro methods of random mutagenesis.

The present invention also provides systems, especially computer systems, arranged to practice the disclosed methods.

Thus the present invention, by use rational, especially computer based and precursor-based, polypeptide design methods which redesign polypeptides to bind to target peptides, the present invention achieves great improvements and efficiencies over prior methods. For example, ab initio CAMD methods are simply too slow and costly to evaluate the millions of possible redesigned polypeptides that need to be evaluated by this invention. On the other hand, random mutagenesis, without direction by rational engineering/design methods, generate thousands of millions and millions of millions of candidate polypeptides that cannot be accurately screened. Similarly, antibody methods, which indirectly reflect largely in vivo mutagenesis unguided by reference to a new antigen, are also error prone. Therefore, it may be difficult, or even not possible, to raise antibodies, especially monoclonal antibodies, in the relevant species to new targets.

Because of these improvements and efficiencies, the present invention achieves the further objects of providing entirely new diagnostic and research applications that take advantage of large numbers of binding proteins (or, in general, macromolecules), each of which is specifically engineered to bind an individual protein in a cell or organism. These diagnostic and research applications, which heretofore were merely speculative, are enabled for the first time by the methods of this invention, which rapidly and economically provide such engineering binding proteins. In a preferred embodiment, these objects are achieved by engineering binding proteins to specifically bind to a short terminal portion of the target protein, for example the terminal 5 to 10 residues at the carboxy-(“C-”) or the amino-(“N-”) terminus, because it has been discovered that such short terminal peptides are sufficient to uniquely identify most proteins of a cell or organism. Binding domains of this invention are preferably engineered entirely without use of, or even knowledge of, of the secondary or tertiary structures of the target proteins, only knowledge of terminal sequences of the new targets being employed by the methods of this invention.

For example, one application now enabled is isolation and purification of arbitrary cellular proteins knowing only a terminal portion of its encoding DNA and nothing about the proteins themselves. A binding domain engineered by the present invention to recognize the terminal sequence encoded by the DNA may be used in standard affinity purification techniques to isolate the target protein for the first time. Previously, when using, e.g., antibodies, sufficient target protein must be first purified to at least allow raising necessary antibodies.

Another now enabled application is the preparation of any number of binding proteins targeted to numerous individual cellular proteins. These binding proteins may be prepared in library formats or in array formats, and used, for example, to screen cellular protein expression. One or more of these libraries and arrays may include 0.5%, 1%, 2%, 3%, 4%, 5%, 10%, 20%, 30%, 40%, 50%, 60%, 70%, 80%, 90% or 95% of the proteins, or of defined classes of proteins, expressed in a cell of an prokaryote or eukaryote or in an entire multicellular organism. In other words, this libraries and arrays, again either singly or as a plurality, may include 500, 1,000, 2,000, 3,000, 4,000, 5,000, 10,000, 20,000, 30,000, 40,000, 50,000, 60,000, 70,000, 80,000, 90,000 or 95,000 binding domains with distinct binding specificity. Defined protein classes may include DNA binding proteins, kinases, phosphatases, signaling pathway proteins, hormones, and so forth. It is particularly preferred to array the binding domains of this invention on a substrate to create a “protein chip” which may be expected to revolutionize proteomic analysis much as “DNA chips” have genomic analysis. These protein chips may be prepared merely from knowledge of the genomic DNA coding sequences, without prior isolation of the target proteins. Preparation of such libraries or arrays with pre-existing antibody technology would require a virtually inconceivable amount of laboratory effort.

In addition to in vitro uses, the engineered binding proteins of this protein also have in vivo uses, especially in that species from which the precursor binding protein originates. For example, the engineered binding domain may be expressed intracellularly and used to localize, or to interfere with the proper localization of, arbitrary proteins in a cell or organism, or they may be expressed as a fusion protein with an existing protein having particular functions in order to endow it with a new intra-cellular targets or properties (such as target protein, rate of degradation, and so forth). This localization can be in living cells or in cells fixed for microscopic investigation. Also, multivalent binding proteins may be formed according to this invention by synthesizing fusion proteins which include two or more engineered binding proteins, either to the same or new different targets. Further, a fusion protein of this invention may include one of more engineered binding proteins along with one or more existing cellular proteins.

The present invention also provides further uses of proteins with engineered binding specificity. For example, binding polypeptides can be labeled by methods routine in the art. Exemplary labeling methods include: conjugating a fluorescent moiety, expressing as a fusion with a protein that can be assayed, being epitope tagged, and so forth.

In more detail, the present invention has the following particular embodiments. In a first embodiment, the invention includes a method of engineering one or more binding macromolecules to adequately bind to a selected target macromolecule, wherein macromolecules comprise linear polymers of monomers, said method comprising: providing, as a first candidate binding macromolecule, a precursor macromolecule which binds to one or more terminal portions of an initial target macromolecule, selecting alternative candidate binding macromolecules by replacing one or more monomers of a current candidate with new monomers, wherein the new monomers are selected by rational engineering methods so that the alternative candidates are predicted to bind with one or more terminal portions of the selected target macromolecule, and wherein the rational engineering methods depend on (i) concerning the selected target macromolecule, input data comprising one or more of its terminal monomer sequences, and (ii) concerning the precursor macromolecule, input data comprising its monomer sequence and the monomer sequences of the terminal portions of the initial target macromolecule bound by the precursor, screening the alternative candidates for new candidates with improved estimated binding to terminal portions of the selected target, wherein the binding is estimated by rational methods in dependence on the input data, and repeating, if necessary, the steps of selecting and screening until the estimated binding of one or more candidates is adequate, whereby one or more candidate macromolecules are engineered to bind to one or more terminal portions of the selected target macromolecule.

In aspects of the first embodiment, the input data concerning the precursor macromolecule comprises its three-dimensional (3D) structure; the input data concerning the selected target macromolecule consists essentially of one or more of its terminal monomer sequences; the monomer are amino acids, and the macromolecules are peptides or polypeptides.

In a second embodiment, the invention includes a method of engineering one or more binding polypeptides to adequately bind to a selected target polypeptide comprising: providing, as a first candidate binding polypeptide, a precursor polypeptide which binds to one or more terminal peptide sequences of an initial target polypeptide, selecting alternative candidate binding polypeptides by replacing one or more amino acid residues of a current candidate with new residues, wherein the new residues are selected by rational engineering methods so that the alternative candidates are predicted to bind with one or more terminal peptide sequences of the selected target polypeptide, and wherein the rational engineering methods depend on (i) concerning the selected target polypeptide, input data comprising one or more of its terminal peptide sequences, and (ii) concerning the precursor polypeptide, input data comprising its amino acid sequence and the terminal peptide sequences of the initial target polypeptide bound by the precursor, screening the alternative candidates for new candidates with improved estimated binding to terminal peptide sequences of the selected target, wherein the binding is estimated by rational methods in dependence on the input data, and repeating, if necessary, the steps of selecting and screening until the estimated binding of one or more candidates is adequate, whereby one or more candidate polypeptides are engineered to bind to one or more terminal peptide sequences of the selected target polypeptide.

In aspects of the second embodiment, the polypeptides comprise peptides, having lengths of less than approximately 20, or 15, or 10, or 5 residues; the input data concerning the precursor polypeptide comprises its three-dimensional (3D) structure; the input data concerning the selected target polypeptide consists essentially of one or more of its terminal peptide sequences.

In further aspects of the second embodiment, the rational engineering or estimating methods for polypeptides comprise methods based on by a priori chemical or physical principles, or are based on rules derived from empirical knowledge, or are based on knowledge in the art; information relating to previously engineered binding polypeptides is stored to supplement the empirical knowledge of the knowledge in the art; the rational engineering or estimating methods that are based on principles comprise one or more computer-assisted molecular design (CAMD) methods for polypeptides; the CAMD methods for polypeptides comprise methods which approximate side-chain conformations by rotamers from a rotamer-library, and which approximate polypeptide backbone conformations by an inverse-folding approach in dependence on a known 3D structure; the CAMD methods may comprise a Perla method. In still further aspects of the second embodiment, the rational engineering or estimating methods that are based on rules further comprise rules derived from examples of sequence homology with known peptide-sequence-binding polypeptides, or derived from examples of polypeptides that bind to peptide sequences, or derived from examples of chimeric polypeptides formed from known peptide-sequence-binding polypeptides; the rules may express peptide-sequence-binding specificities of peptide-sequence-binding polypeptides; the rules may express how peptide-sequence-binding specificities of polypeptides may be modified; the rational engineering or estimating methods that are based on common knowledge further comprise classification of the amino acids into types with similar physical and chemical properties.

In a yet further aspect of the second embodiment, the precursor polypeptide binds to two or more terminal peptide sequences of the initial target polypeptide, and wherein the step of repeating, if necessary, repeats the steps of selecting and screening until the estimated binding of one or more candidates to two or more terminal peptide sequences of the selected target polypeptide is adequate.

In a third embodiment, the invention includes a method of engineering one or more binding polypeptides to adequately bind to a selected target polypeptide, wherein a peptide sequence has a length of less than approximately 20, or 15, or 10, or 5 residues, the method comprising: providing, as a first candidate binding polypeptide, a precursor polypeptide which binds to one or more N-terminal peptide sequences of an initial target polypeptide, selecting alternative candidate binding polypeptides by replacing one or more amino acid residues of a current candidate with new residues, screening the alternative candidates for new candidates with improved binding to N-terminal peptide sequences of the selected target polypeptide, and repeating, if necessary, the steps of selecting and screening until the binding of one or more candidates is adequate.

In aspects of the third embodiment, the steps of selecting and screening further comprise rational engineering or estimation methods for polypeptides that are based on by a priori chemical or physical principles, or that are based on rules derived from empirical knowledge, or that are based on knowledge in the art; and the precursor polypeptide has a known three-dimensional (3D) structure.

In a fourth embodiment, the invention includes a computer system for engineering one or more binding polypeptides from a selected precursor polypeptide, wherein the precursor polypeptide binds to one or more terminal peptide sequences of an initial target polypeptide, and wherein the binding polypeptides adequately bind to a selected target polypeptide, the system comprising: a processor, and a memory accessible to the processor, wherein the memory is configured with (a) data for representing the precursor polypeptide, the initial target polypeptide, the selected target polypeptide, and further candidate polypeptides, and wherein (i) the data for representing the selected target polypeptide comprises data representing one or more of its terminal peptide sequences, and (ii) the data representing the precursor polypeptide comprises data representing its amino acid sequence and the terminal peptide sequences of the initial target polypeptide bound by the precursor, and (b) instructions for causing the processor, in dependence on the represented data, to perform the steps of (i) rational engineering methods for selecting alternative candidate binding polypeptides by replacing one or more amino acid residues of a current candidate with new residues so that the alternative candidates are predicted to bind with one or more terminal peptide sequences of the selected target polypeptide, (ii) rational binding-estimating methods for screening the alternative candidates for new candidates with improved estimated binding to terminal peptide sequences of the selected target, and (iii) repeating, if necessary, the steps of rational engineering and estimating until the estimated binding of one or more candidates is adequate, whereby one or more candidate polypeptides are engineered to bind to one or more terminal peptide sequences of the selected target polypeptide.

In aspects of the fourth embodiment, the instructions for causing the processor to perform the steps of rational engineering or of rational binding-estimating comprise instructions for performing methods that are based on a priori chemical or physical principles, or that are based on rules derived from empirical knowledge, or that are based on knowledge in the art; the methods that are based on principles further comprise one or more computer-assisted molecular design (CAMD) methods for polypeptides; the CAMD methods for polypeptides comprise methods which approximate side-chain conformations by rotamers from a rotamer-library, and which approximate polypeptide backbone conformations by an inverse-folding approach in dependence on a known 3D structure; the CAMD methods comprise a Perla method; the instructions for causing the processor to perform a CAMD method comprise instructions for performing two or more CAMD methods of increasing accuracy.

In a fifth embodiment, the invention includes a computer-readable media with encoded instructions stored therein for causing a computer to perform the method of the fourth embodiment.

In a sixth embodiment, the invention includes a polypeptide for binding to a selected target polypeptide engineered according to the method of the second embodiment.

In a seventh embodiment, the invention includes a vector for causing expression in a host cell of a polypeptide engineered for binding to a selected target polypeptide according to the method of the second embodiment.

In an eighth embodiment, the invention includes a cell comprising a nucleic acid sequence encoding a polypeptide engineered for binding to a selected target polypeptide according to the method of the second embodiment. In aspects of the eighth embodiment, the engineered polypeptide is fused to a partner polypeptide comprising a peptide or a polypeptide; the partner polypeptide may comprise a polypeptide sequence causing the localization of the fusion to a selected intracellular compartment; the partner polypeptide may comprise a polypeptide sequence causing degradation of the fusion; the partner polypeptide may comprise a label.

In a ninth embodiment, the invention includes a method for altering the function of a first cellular protein, which does not naturally bind to a second cellular protein, comprising: providing a binding protein engineered according to the method of the second embodiment for binding to the second cellular protein, and expressing the binding protein fused to the first cellular protein so that first cellular protein as part of the fusion binds non-naturally to the second cellular protein.

In a tenth embodiment, the invention includes a cell comprising a nucleic acid encoding a cellular protein altered according to the ninth embodiment.

In an eleventh embodiment, the invention includes a method for altering the function of a selected cellular protein, which naturally binds to an initial polypeptide, comprising: engineering the selected cellular protein to bind to a new target polypeptide according to the method of the second embodiment, and expressing the engineered selected cellular protein.

In a twelfth embodiment, the invention includes a method for assaying for one or more target polypeptides in a sample comprising: contacting, in binding conditions, the sample with binding polypeptides, wherein one or more binding polypeptides are engineered by the method of the second embodiment to bind to one or more of the target polypeptides, and assaying for binding polypeptides bound to their respective target polypeptides, whereby the target polypeptides are assayed. In an aspect of the twelfth embodiment, one or more binding polypeptides are attached to a substrate.

In a thirteenth embodiment, the invention includes a method of determining the cellular localization of a target protein comprising: providing a binding protein engineered by the method of the second embodiment to bind to the target protein, contacting the cell with the binding protein under binding conditions, and assaying for the presence and location in the cell of the binding protein bound to the target protein.

In a fourteenth embodiment, the invention includes a method for assaying for target proteins in a sample from an organism comprising: contacting, in binding conditions, the sample with binding polypeptides, wherein the binding polypeptides bind to one or more terminal peptide sequences of a plurality of selected proteins expressed in the organism, and wherein the plurality of selected expressed proteins comprises more than 50 different proteins, and assaying for binding polypeptides bound to their respective target proteins, whereby the target proteins are assayed.

In aspects of the fourteenth embodiment, the binding proteins are engineered to bind to the terminal peptide sequences of the selected plurality of proteins by the method of the second embodiment; and the binding polypeptides are attached to one or more substrates. In further aspects of the fourteenth embodiment, the plurality of selected proteins comprises more than 500 or more than 5,000 different proteins; or the plurality of selected proteins comprises less than 5,000 or less than 50,000 different proteins; or the plurality of selected proteins comprises more than 0.5% of the proteins expressed in a cell of the organism; or the plurality of selected proteins comprises less than 50% or less than 80% of the proteins expressed in a cell of the organism.

In a fifteenth embodiment, the invention includes a library comprising recombinant entities expressing a plurality of binding polypeptides, wherein each binding polypeptide binds to one or more terminal peptide sequences of each of a plurality of selected proteins expressed in an organism, and wherein the plurality of selected expressed proteins comprises more than 50 different proteins.

In aspects of the fifteenth embodiment the plurality of selected proteins comprises more than 500 or more than 5,000 different proteins; or the plurality of selected proteins comprises less than 5,000 or less than 50,000 different proteins; or the plurality of selected proteins comprises more than 0.5% of the proteins expressed in a cell of the organism; or the plurality of selected proteins comprises less than 50% or less than 80% of the proteins expressed in a cell of the organism.

In a sixteenth the invention includes a polypeptide array comprising a substrate with at least one surface, and a plurality of binding polypeptides regularly arranged on the surface, wherein each binding polypeptide binds to one or more terminal peptide sequences of each of a plurality of selected proteins expressed in an organism, and wherein the plurality of selected expressed proteins comprises more than 50 different proteins.

In aspects of the sixteenth embodiment the binding polypeptides are covalently attached to the surface; the substrate comprises glass or plastic; the terminal peptide sequences are N-terminal sequences, or C-terminal sequences, or both, having lengths less than approximately 15 amino acids. In further aspects of the fifteenth embodiment the plurality of selected proteins comprises more than 500 or more than 5,000 different proteins; or the plurality of selected proteins comprises less than 5,000 or less than 50,000 different proteins; or the plurality of selected proteins comprises more than 0.5% of the proteins expressed in a cell of the organism; or the plurality of selected proteins comprises less than 50% or less than 80% of the proteins expressed in a cell of the organism.

In a seventeenth embodiment, the invention includes a polypeptide-RNA-fusion array comprising a substrate with at least one surface, and a plurality of binding-polypeptide-RNA fusions regularly arranged on the surface, wherein each binding polypeptide of the fusions bind to one or more terminal peptide sequences of each of a plurality of selected proteins, and wherein the RNAs of the fusions comprise sequences that encode for the corresponding fused binding polypeptides.

In an eighteenth embodiment, the invention includes a method of purifying one or more selected proteins from a sample comprising: providing one or more binding polypeptides that bind to the terminal peptide sequences of a one or more selected proteins, wherein the binding polypeptides are engineered by the method of the second embodiment, contacting the sample in binding conditions with the binding polypeptides so that selected proteins in the contacted sample are bound to the binding proteins, washing the contacted sample in washing conditions so that unbound proteins are removed while bound selected proteins remain, and eluting the washed sample in eluting conditions so that bound selected proteins are removed from the binding polypeptides, whereby the eluted selected proteins are purified from the sample.

### BRIEF DESCRIPTION OF THE FIGURES

The present invention may be understood more fully by reference to the following detailed description of the preferred embodiment of the present invention, illustrative examples of specific embodiments of the invention and the appended figures in which:

FIG. 1 illustrates an embodiment of the methods of the present invention.

FIGS. 2A-C illustrate a first exemplary precursor protein domain.

FIGS. 3A-B illustrate a second exemplary precursor protein domain.

FIGS. 4A-B illustrate a third exemplary precursor protein domain.

FIG. 5 illustrates exemplary systems of the present invention.

FIG. 6 illustrates a schematic description of the preferred computer-assisted molecular design software, known as Perla.

FIG. 7 illustrates plasmid pQEPDZ3, a plasmid containing a fusion between the third PDZ domain of PSD95 (amino acid 302-402) and the polyhistidine (6XHis).

FIG. 8 illustrates interactions between different PDZ domains and target peptides revealed by two-hybrid analysis. The plate labeled Eg5B contains the following results: (A) PDZEg5+EGFP; (B) PDZEb5+EGFP-tub; (C) PDZEg5+EGFP-pep; (D) PDZEg5+GalBd; (E) PDZEg5+EGFP-Eg5 and (F) PDZ-3+EGFP-eg5. Only the PDZEg5+EGFP-Eg5 combination results in viable cells showing that the re-designed PDZEg5 domain specifically interacts with its target peptide present in the EGFPP-Eg5 fusion protein. The plate labeled PDZ-3 contains the following results: (A) PDZ-3+EGFP; (B) PDZ-3+EGFP-tub; (C) PDZ-3EGFP-Eg5: (D) GaLAD+GalBD; (E) PDZ-3+EGFP-pep; (F) PDZ-3+EGFP-pep. This is the positive control showing that the PDZ-3 domain specifically interacts with its target peptide present in the EGFP-pep fusion protein.

FIG. 9 illustrates affinity chromatography. The original PDZ domain (PDZ-3) and the redesigned PDZ (PDZ-Eg5) were immobilized in a solid phase and their efficiency to bind non-modified GFP (A) and GFP fused to either the C-terminal peptide recognized by the original PDZ (B) or the C-terminal peptide of Eg-5 (C) was determined by Western Blot. PDZ-3 binds only to its naturally recognized target peptide. The re-designed PDZ-Eg5 binds only to the Eg-5 C-terminal peptide.

FIG. 10 illustrates sub-cellular localization of a protein fusion made of GFP and a PDZ domain that had been engineered to recognize the centrosome-associated protein Eg5. The redesigned PDZ domain that recognizes the C-terminus of Eg5 (PDZ-Eg5B) fused to GFP can be seen to accumulate around the microtubule organizing center where Eg5 is located (See, e.g., Cell 83:1159-1169 (1995); J Neurosciences 18:7822-7835 (1998)).

FIG. 11 illustrates combining two separate PCR products with overlapping sequence into one longer product. The two overlapping primers are shown containing a mismatched base to the target sequence.

FIG. 12 illustrates using inside primers for the creation of deletions (A) or small insertions (B).

FIG. 13 illustrates recombinant PCR. Primers and sequences are shown for the joining of gene and promoter sequences.

FIG. 14 illustrates determination of the affinity of a domain to a target by means of micro-calorimetry.

FIG. 15 illustrates the results of methods of the present invention applied to re-design a wild type PDZ domain (designated PDZ-Wt*) to bind to its natural target, the last nine amino acids of the protein CRIPT (designated as “pep”). In (A), the re-designed domain (PDZ-Wt*) is bound to a substrate for affinity purification of GFP (green fluorescent protein) alone (lane labeled “GFP”), GFP fused with last nine amino acids of CRIPT (lane labeled “GFP-Pep”), and GFP fused with the last nine amino acids of eg5 Kinesin (lane labeled “GFP-eg5”). (A) demonstrates that PDZ-Wt* binds only to GFP-pep. In (B), two-hybrid assay results of PDZ-Wt* fused to the activation domain of gal4 (labeled pGAD wt*) with different GFP-fusions: “pGBKT7-GFP” designating GFP fused to the binding domain of GAL4; “pGBKT7-GFP-pep” designating GFP-pep fused to the binding domain of GAL4; and pGBKT7-GFP-eg5 designating GFP-eg5 fused to the binding domain of GAL4. (B) demonstrates cell viability occurs only when pGAD wt* binds to pGBKT7-GFP-pep.

### DETAILED DESCRIPTION OF THE PREFERRED EMBODIMENT

The detailed description of the present invention is presented herein, where: Sec. 5.1 describes domain engineering/redesign methods; Sec. 5.2 describes screening and selection of candidate domains; Sec. 5.3 describes synthesis of engineered domains; Sec. 5.4 describes systems for practicing the invention; and Sec. 5.5 describes uses of engineered binding domains; Sec. 5.6 describes other embodiments and aspects of the invention. Sec. 6 presents examples of the methods of this invention.

### Domain Engineering/Redesign Methods

Before describing domain engineering methods of this invention, preferred structures of precursor domains are described.

### Preferred Precursor Domains

As is conventionally known, macromolecules, especially preferred linear polypeptides and polynucleotides, are characterized by the hierarchy of their primary, secondary, and tertiary structures. (Quaternary structures are not generally of interest in the present invention; but see protein PDB id. 1IDP subsequently.) Primary structure is the sequence of monomer building blocks, for example, the amino acid sequence of a protein. Secondary structures are organized spatial structures formed by several monomers along the molecular backbone. In the case of proteins, α-helices and β-sheets are well known and important secondary structures. Especially in proteins, two or more secondary structures adjacent along the backbone can form organized “super-secondary” spatial structures, also sometimes known as motifs, such as β-hairpins or helix-turn-helices. Finally, tertiary structure describes the overall folding of the secondary structures of a protein into its overall three-dimensional (3D) conformation stable at physiologic conditions. Tertiary structures can include spatially adjacent secondary structures which are far apart in the primary sequence.

Although discrete tertiary (and assemblies of super-secondary) structures may be referred to as “domains”, especially using functional classification (proteinases, transcription factors, and so forth), the term “domain” is taken herein in a distinct and precise sense. As used herein, a “domain”, either an initial “precursor domain” or an engineered/redesigned “binding domain”, is macromolecule that binds to selected target macromolecule and has stable secondary and tertiary structure sufficient so that binding specificities may be engineered by replacement of a few monomer building blocks (preferably, less than 5 or 10 or 15). In preferred embodiments, the binding macromolecules are proteins (or generally polypeptides having optional non-natural amino acids), and the selected target macromolecules are preferably proteins, or polypeptides, or peptides. In more preferred embodiments, the selected targets are terminal (either the C- or the N-terminus) peptide sequences of naturally-occurring proteins. Herein, a “peptide” is a short polypeptide molecule that is devoid of fixed structure in water (in the absence of tertiary contacts), while a “peptide sequence” is a contiguous short portion of a polypeptide. Peptide sequences are typically less than about 20 residues, or less than about 15 residues, or preferably less than about 10 residues, or less than about 5 residues.

Preferred protein, or polypeptide, or peptide domains may have further advantageous properties. Such domains are preferably water soluble and generally largely globular with a solvent-accessible surface on which the target peptide sequence binds, preferably the domain surface present no steric hindrances that would prevent binding of a terminal peptide sequence of a larger protein or polypeptide. Preferably, the peptide binds in a substantially extended configuration in a groove, pocket, cleft or other spatial structure (“binding region” or “binding pocket”) which is in proximity to the peptide backbone, or to the protein's amino acid side chains, or to both. The binding region/pocket may typically be defined by secondary structures which are spatially adjacent to a bound target peptide. Because of this proximity, the peptide interacts with the domain by hydrogen bonds, electrostatic potentials (including charge and dipole interactions), van der Waals forces, hydrophobic interactions (such as hydrophobic pockets and surfaces), and other non-bonding interactions (generally referred to as “contacts”). Such contacts may arise from the residues in two or more secondary structures of the domain adjacent to two or more sides of the target peptide, for example, two α-helices laterally adjacent to the bound peptide. Advantageously, contacts between the domain and the target peptide sequence encourages peptide binding in a generally extended configuration (generally linear without secondary structures). Contacts between the domain and the amino acid side chains of the target peptide sequence, which are easily accessible in an extended conformation, encourage sequence specificity of binding. Further, it is preferred that precursor domains for in vivo intracellular use have no disulfide bridges, which may prevent intra-cellular use. For in vitro extracellular use, disulfide bridges may advantageously provide extra stability to a precursor.

For a specific example, where a C-terminal peptide sequence of a protein is bound, the domain preferably includes (either naturally or after engineering) positively-charged amine groups and/or hydrogen-bond-donor residues to interact with the negatively-charged target C-terminal hydrogen-bond-accepting carboxy groups. Conversely, where an N-terminal peptide sequence is bound, the domain should contain negatively-charged carboxyl groups and/or hydrogen-bond acceptors to interact with the target positively-charged hydrogen-bond-donor N-terminal amine groups. Where the target peptide has one or more hydrophobic amino acid residues, the domain should present an adjacent hydrophobic region or hydrophobic cleft to sequester the hydrophobic peptide residues out of solvent contact. These domain residues should be suitably oriented towards the interior of the binding region and not have hydrogen bond partners elsewhere in the protein.

Preferably, the non-covalent bonding between the domain and a bound peptide should be sufficient so that the free-energy of binding leads to a dissociation constant (K_{d}) of preferably less than about 1 mM, or more preferably less than about 100 μM, or less than about 1 μM or 500 nM, or smaller.

Therefore, engineering the binding specificity of a binding domain protein may lead to replacing or substituting or mutating between 10 and 20 amino acid residues of the domain. It is preferable for the methods of this invention that such replacement or substitutions not substantially disturb existing secondary and tertiary structures, which bring binding domain residues into geometric arrangements for interacting with residues of the bound peptide. Stated differently, it is preferable that engineered amino acid substitutions or mutations alter the spatial configuration of the domain's peptide backbone by approximately 1 Å (10⁻⁸ cm) or less (on average), or at least not substantially disturb the spatial configuration of the backbone structures defining the binding region or pocket. This stability is preferable it limits the number of residue substitutions or mutations that must be examined to those in the vicinity on the target peptide sequence only.

One rule-of-thumb for substantially preserving existing secondary and tertiary structure of a binding domain is that the number of substituted or changed or mutated amino acid residues be less than about 10% (or less preferably 20%) of the total number of residues of the binding domain protein. The unchanged residues then may provide sufficient stabilizing intra-molecular interactions to maintain overall spatial structures. Thus a preferred binding domain has a total number of residues exceeding 5, 10, 40, or 100, or 200; and in particular exceeding 50, 60, 70, 80, 90, 100, 110, 120, 130, 140, 150, 160, 180, or even 200 so that approximately 5, or 10, or 20 residues may be available for substitution. However, these rules-of-thumb are not limiting, what is preferable is only that the substitutions or changes, however numerous, do not substantially disturb (less than about 1 Å on average) existing secondary and tertiary structures (at least do not disturb existing secondary and tertiary structures to the extent that the binding region of the domain is substantially disturbed).

Precursor binding domains satisfying most or substantially all of the above preferences can be readily and routinely determined from the rapidly accumulating data on the functional and structural properties of naturally occurring proteins. This data is becoming available in various publically accessible databases. For example, the secondary and tertiary structures of proteins already known to selectively bind to peptides can be routinely determined in sufficient resolution (for example approximately 2×10⁻⁸ cm or less) by the methods of X-ray crystallography (“XRD”) and nuclear magnetic resonance (“NMR”), both free and as bound to their target. It is less preferable, though possible, for these spatial structures to be entirely determined by molecular modeling methods. From these available structures, more preferred precursor domains or proteins can be routinely determined by inspection.

One of skill in the art will recognize that the invention is not limited to these preferred domains, but that is methods readily apply to domains within the general meaning set forth above.

### General Engineering/Redesign Methods

The methods of the present invention rapidly and economically engineer or redesign preferred precursor domains of known binding specificity (as described above) into engineered/redesigned binding domains capable of specifically and selectively binding to a broad range of selected smaller macromolecule targets, either a free smaller macromolecule or a smaller portion of a larger macromolecule. Smaller macromolecules refer here to those macromolecules built from a small number of building blocks, for example, from 1 to 10 (to 15 or 20) building blocks. Because naturally-occurring macromolecules do not have random sequences, they may often be individually identified by knowing sequences of short portions. Therefore, by being able to specifically bind smaller portions of larger macromolecules, this invention actually provides specific binding domains for an immense number of naturally-occurring macromolecules, in particular for recognizing a substantial fraction of all the proteins in the cells of a species, or all the proteins of selected types, such as phosphatases, kinases, signaling proteins, and so forth. And because of its efficiency and economy, this invention also provides these engineered binding domains for the first time in large numbers.

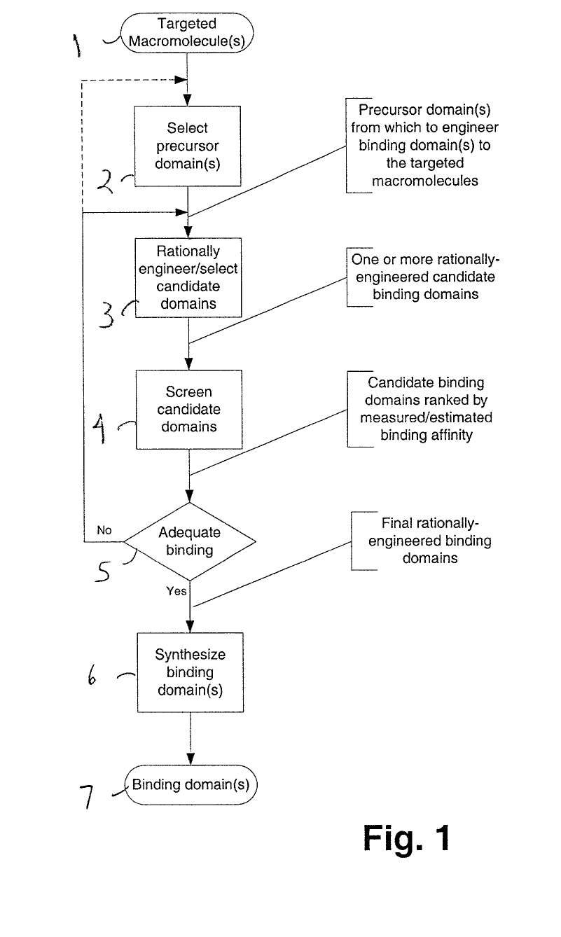

FIG. 1 illustrates an embodiment of the domain engineering/redesign methods of the present invention. Binding domain design begins with the identification in step 1 of the one or more targeted macromolecules, which can be, inter alia, peptides, proteins, polynucleotides, polysaccharides, lipids, or other natural polymers. (Also, polymers with non-naturally occurring monomer building blocks, or entirely non-naturally occurring classes of polymers, may be targeted.) It is advantageous, in order to achieve particular benefit of this invention, that the targeted smaller macromolecules, or the targeted smaller portions of larger macromolecules, have substantially no secondary, tertiary or higher structures at the temperature of interest (particularly, structures that are stable enough to prevent being bound to a binding domain in a substantially extended conformation). Instead, it is preferable that they may readily flex into alternative conformations at small energetic cost (in comparison to free energies of binding necessary to achieve desired binding affinities and the temperature).

Step 2 selects one or more precursor domains from which to engineer/redesign new binding domains specific to the new molecular targets. Preferable precursor domains are macromolecules which are known to bind to at least one macromolecule of the same type as the targeted macromolecule, i.e., a precursor for targeted peptide sequences of a certain length preferably binds at least one peptide sequence of similar length as described above. At the completion of these steps, one or more targeted macromolecules have been selected along with one or more precursor domains to be engineered to bind to the targeted macromolecules.

Engineering/redesign of the precursor domains selected in step 2 is preferably performed by a nested and iterative “select and test” process that is illustrated in steps 3, 4, and 5. Generally, step 3 provides a plurality of candidate domains for binding to the new target that are derived from the precursor domain, or from the candidate domains resulting from a prior “select and test” iteration, by replacements of amino acid residues with different “mutated” residues chosen according to selected criteria or methods to likely result in better binding on average to the new target (an “improved” binding domain). The criteria and methods employed in this step, according to the present invention, are based on principles of “rational design/selection”. Briefly, “rational design” principles derive from physical and chemical principles that are used in approximate form in computer-based modeling, or from databases of known protein structures that are used in homology modeling, sequence alignment, and the like, or from empirical rules derived from examples of polypeptide-ligand binding known in the art, of from common chemical knowledge (such rules also including tables of amino acid properties, such as indices of hydrophobicity), or so forth. Additionally, rational design principles may derive from sequence comparisons and mutation preferences of certain residue positions (such as residues defining a binding pocket), or from covariance analysis of mutations between a precursor protein and its natural targets. In general, these principles may be based on any approach where a rational decision (as opposed to a random or unguided decision) to select mutations at a small number of residues in a target or precursor protein is used in order to modify its properties.

Rational design does not include laboratory techniques, such as random mutagenesis, which are unguided by a priori principles, or knowledge, or rules. Such random and unguided techniques cannot choose candidates which on average are likely to result in improved candidates with better binding; instead such “non-rational design/selection” methods rely on subsequent screening to select improved candidate domains. Rational design/selection is often performed by computer-implemented methods (otherwise known in the art as in silico methods) operating on data representing the precursor domain or the candidate domains to the extent possible and reasonable for a particular engineering problem. But even visual inspection, aided by well-known polypeptide visualization tools and guided by empirical polypeptide-ligand binding rules and chemical knowledge common in the art, of a few (5 or 10) candidates demonstrating superior binding in a prior CAMD method may be used in a final selection step.

In more detail, a first rational selection iteration may use less-accurate rational selection tools that are nevertheless fast, easy to apply, and capable of exploring larger candidate-domain spaces. These tools include more approximate and less accurate but faster CAMD methods, or empirical rules derived from known examples, or sequence alignment and homology to other precursor domains with different target specificities, or chimeric modeling, or so forth. A second iteration may then explore the possible candidates selected from this larger space by using slower but more accurate CAMD tools to select the one or more highly promising candidates. In the case of proteins, tools for the first sub-step could use rapid geometric considerations and a rotamer library (see subsequent) to select candidate binding domains, the residues of which have sufficient contacts with a target peptide for binding affinity, and discard those candidates that have steric hindrances or insufficient contacts with a target peptide. Tools for the second sub-step could then explore the detailed binding of selected candidates by, for example, also considering limited perturbations of the domain backbone. Such an iterative use of CAMD tools may improve both breadth and accuracy sufficient to permit elimination of screening step 4, as suggested above. Further alternatives will be immediately apparent to one of skill in the art in view of the subsequent description, and the present invention is intended to encompass such alternatives.

Step 4 screens the chosen “improved” binding domains. The tools used for this screening preferably depend on the rational design methods used in the previous iteration of step 3. For example, where the previous rational selection was made by rules known to those of skill in the art, the subsequent screening step may be a CAMD process which uses a computer program based on physical and chemical principles to evaluate the binding of the chosen candidates to the new target. Where the previous rational selection was an accurate CAMD step resulting in a few candidates, the subsequent screening step may involve actual laboratory determination of affinity. Also, where the previous rational selection was a accurate CAMD process known to be reliably and predicably accurate, the subsequent screening step may be omitted. In other words, step 4 is not limited to laboratory screening using yeast two-hybrid, phase-display, or other known screening methods. Instead, a subsequent iteration of the screening step may also be depend on a priori principles, or knowledge, or rules not requiring laboratory determination. It is preferable that screening step be more certain, or more accurate, or more reliable than the previous selection step so that the iterative “select and test” process progresses to candidates of steadily increased binding affinity.

Step 5, then, completes the “select and test” process, returning for another iteration when test-step 4 does not indicate adequate binding. It is preferable that step 5 only signal final adequate binding and pass to step 6 when the screening involves laboratory methods.

Steps 3, 4, 5 and may also be advantageously viewed and performed as a search process. For example, the search space may include candidates generated by selecting one or more sites along the precursor domain macromolecule for mutation and by selecting one or more alternative building block monomers at each selected site, each alternative candidate in the search space differing from the precursor domain by having mutated monomers at selected sites. Sites and mutated monomers may be selected by rational design methods, for example, by rules known in the art, so that the generated domains are a priori more likely than not to be an acceptable candidates, or alternatively are predicted to bind with some, although small affinity (e.g., 100 mM, or 1000 mM, or greater dissociation constant). This candidate-domain space is then searched for those candidates that optimize an objective function, such as a predicted binding of a candidate domain to the targeted macromolecule. Preferably, domain binding may be predicted by CAMD or other computer-based rational techniques. In one embodiment, candidate domains in the space may first be generated, and second, the binding of all the domains then predicted. In another embodiment, as soon as a candidate domain is generated, its binding may be predicted. In this latter embodiment, search heuristics known in the computer arts may be used to prevent the domain space search from pursuing branches known to not contain optimal domains.

Preferably, step 3 selects a limited number of candidates, more preferably 1, but also preferably approximately 10, approximately 100, approximately 1,000, or so promising candidate domains so that the screening of step 4 can be focused and efficient with a minimum errors. This is especially preferably where laboratory methods are used for screening. For example, a limited number of candidates permit construction of small constrained libraries, greatly increasing the probability of finding a binding domain. Only selected precursor residues need be mutated in a restricted fashion. In contrast, prior chemical or biological mutagenesis techniques that lead to random libraries having unrestricted mutations at many positions, including those entirely irrelevant to target binding, are so large, approximately 10⁶ to approximately 10¹⁰ or more members, that their screening is difficult, time consuming, and most likely to false negative errors and to mutations compromising the viability or stability of the precursor protein (i.e., missing promising candidate domains and reducing binding domains even if present).

A final iteration of step 4 screens the most promising candidate binding domains for actual binding to the targeted macromolecule. The candidate binding domains are synthesized by known techniques, for example by recombinant technology, and used in various known assays for macromolecular interaction, for example the yeast two-hybrid assay. (If a candidate binding domain includes a non-natural monomer building block, such as a non-natural amino acid, then chemical synthesis may be necessary.)

Step 5 then determines whether the assay results demonstrate adequate binding. Adequate binding may be determined by selection criteria, including for example, semi-qualitative inspection of assay results, or determination of approximate affinity or dissociation constants, or screening for false-positive binding to non-targeted macromolecules, or confirmation of intended binding domain function, or so forth. An adequately engineered binding domain may be one that demonstrates adequate binding affinity with a substantial absence of false-positive binding to non-targeted macromolecules.

If one of more of the candidate binding domain meets the step-5 criteria, then they can be synthesized in step 6, usually by recombinant technologies but also direct chemical synthesis, and then used in step 7 for their intended purposes. Step 6 may also include intra-cellular expression alone or as part of a fusion protein. On the other hand, if no candidate binding domains meet the step-5 criteria, or if a final screening is not yet complete then steps 3 and 4 may be repeated with rational selection principles that are preferably more accurate. Indeed, in after several iterations, candidate binding is not improving, the present invention contemplates returning to step 2 to select additional precursor domains. Or a more accurate but possibly more time consuming CAMD, or other rational selection, techniques may be used to explore the original or an enlarged space of candidate domains.

In the preferred embodiment of the present invention, precursor domains are proteins, either naturally occurring or artificially synthesized in whole or in part (perhaps including non-naturally occurring amino acids). Proteins are attractive precursors, because they are known to be capable of binding a broad range of targets, from small molecules to biological macromolecules of all types, and because extensive prior work on protein structure and engineering has resulted in many modeling techniques useful in this invention, for example, numerous rational selection tools, such as numerous CAMD techniques, empirically derived rules, and so forth. In a further preferred embodiment, the targeted molecules are peptides, possibly peptides modified by, inter alia, phosphorylation or glycosylation or including non-naturally occurring amino acids. Not only are short peptides generally flexible at physiological temperatures, but also as discussed above and below, recognition of peptides permits targeting of a substantial fraction of, or substantially all of, the proteins naturally occurring in an organism. This ability has great use, at least, in the fields of medicine, biology and biochemistry, similar to vital advances due to the ready availability of artificial polynucleotides complementary to, and thus recognizing, virtually any naturally occurring polynucleotide.

Finally, it is noted that important efficiencies of this invention derive at least in part from the iterative “select and test” loop in which rational selection techniques, such as CAMD techniques, are repeatedly used before more cumbersome and expensive laboratory techniques. Thus, combinatorially-large spaces of alternative, candidate domains may be searched rapidly and efficiently in silico (that is by computer-based methods) without any need for in vitro or in vivo experimentation. Only when a few particularly promising candidate domains have been identified are actual in vitro or in vivo binding assays performed, if they need be performed at all. Such assays, screening only a few candidates, are inherently less error prone that previous methods which necessarily had to screen very large random libraries. This efficiency leads to the surprising result that this invention now makes possible selective binding domains targeted to a substantial fraction, substantially all, or all the proteins of a selected class, or of all proteins, expressed in the cells of an organism. Therefore, this invention makes possible new directions in the field of proteomics, such as by providing for the simultaneous and parallel screening for a substantial fraction of all the proteins expressed in a cell.

In the following, although the present invention is primarily described in terms of its preferred embodiment, this description is not intended to be, and will be recognized by one of skill in the art not to be limiting. FIG. 1 illustrates but one embodiment of the present invention. The selection of precursor domains in step 2 can be eliminated if one or more precursor domains suitable to the targeted macromolecules are already known. As already described, the “select and test” steps, steps 3, 4, and 5, have many embodiments. Step 6 may be eliminated if adequate amounts of the binding domain have already been synthesized in connection with screening step 4. Further, the principles inventively employed to rationally engineer protein precursors are immediately applicable to engineer precursor macromolecules of other types that, at least, have regular structures capable of limited, controlled and predictable modifications. Further, the principles employed are also applicable to engineer precursor domains of one type to bind a macromolecule of a different type or to bind a macromolecules of a type different from its natural target. Additionally, it will be apparent that this invention can be applied to a precursor domain binding a small (non-polymeric) molecule to bind another structurally-related small molecule.

This invention is now described in more detail in its preferred embodiments applied to rationally engineering polypeptides binding domains targeted to short peptide sequences, which are preferably C- or N-termini of proteins. This embodiment has the varied utilities already discussed.

### Preferred Targets

Short peptide sequences, of from 2 to 10, and also up 15 to or up to 20, residues, are preferred targets, because they may generally be bound in an extended conformation (or other non-hindered conformations). Any secondary spatial structures short peptides may assume free in solution easily alters to an extended bound conformation in physiological condition with at most a small expenditure of free energy (sufficiently small compared to their free energy of binding that desired binding affinities are not substantially disturbed). Therefore, only short amino acid sequences need be considered (along with the substantially fixed backbone structure of the precursor protein domain), which greatly simplifies CAMD and other rational selection techniques. See, e.g., Cregut et al., 1999, J. Mol. Biol. 292:389-401; Minor et al., 1999, Nature 380-730-735.

Alternatively, selected target macromolecules may have spatial structures that may be accurately predicted from their primary structures with little effort (compared to exploring candidate domains). For example, the structures of A-DNA of B-DNA are of this type. In this case, the primary structure of the short sequence determines the secondary structures than must be bound by an engineered domain.

As described, preferred precursor protein domains are those whose secondary or higher order spatial structures are substantially independent of the binding of their natural target, which is a short peptide sequence. This structure stability may readily be ascertained by comparing experimentally determined structures for the free precursor domain with structures for the precursor domain bound to its natural target. Also, if the residues in contact with the bound peptide are embedded in a larger enclosing polypeptide chain which possesses secondary, tertiary, and high-order structures stable under physiological conditions, then engineering the amino acids responsible for target binding may be expected to have little or substantially no effect of higher order structures of the enclosing protein, their structure being primarily fixed by the surrounding three-dimensional environment. See, e.g., Baker et al., 1996, Folding & Design 1:R71-R77. This is advantageous because the spatial structure of the precursor protein need only be determined once, by experimental or ab initio means. Then subsequent rational design techniques, such as CAMD techniques, may approximate the binding-domain backbone as substantially fixed (being perturbed by less than about 10⁻⁸ cm). Alternatively, precursor domains may have substantially stable spatial structure because they include peptide analogues or peptides “scaffolded” by alternative means, for example, by disulfide bonds, to other polypeptides or to an external scaffold (known as “template-assisted design”).

Targeting peptide sequences is of broad usefulness, because recognition it permits ultimate targeting most, or nearly all, of the naturally occurring proteins in a cell or organism. First, sequence studies of the N-terminus, or C-terminus, or both, of proteins mycoplasma genitalium, bacillus subtilis, escherichia coli, saccharomyces cerevisiae and of humans, show that these termini are highly specific. Depending on the species, N-terminal peptides of only four amino acid residues uniquely identify between 43% and 83% of proteins, and C-terminal peptides also of only four amino acid residues uniquely identify between 74% and 97% of proteins. Sequence tags of five amino acid residues are even more specific. See e.g., Wilkins et al., 1996, Protein identification with N- and C-terminal sequence tags in proteome projects, J. Mol. Biol. 257:175-187. If all 20 amino acids were randomly present, terminal peptide sequences of 4 to 5 residues would suffice to identify approximately 10⁵ to 10⁶ proteins, the order of magnitude of the estimated number of human expressed proteins. Random terminal peptide sequences of 7 residues would suffice to identify approximately 10⁹ proteins. Thus, targets are preferably short peptide sequences of at least 4-5 residues long, more preferably 6-7 residues, and even more preferably 8-10 (or more) residues.

In addition to being specific, terminal peptides are usually accessible in a generally extended conformation (or at least a conformation determined by the binding domain) for binding with a binding domain. Most protein structures solved to date indicate that the N- and C-termini tend to be solvent-exposed, to have little stable secondary structure, adopting a rather extended conformation and having little interaction with or contacts to the rest of the protein structure. See, e.g., Thornton et al., 1997, Amino and carboxy-terminal regions in globular proteins, FEBS Lett 404:140-2. Therefore, they can be bound by domains in dependence on only their primary structure. Further, binding terminal peptide sequences should interfere only minimally, if at all, with the protein's normal biological function, or life cycle (except when these termini are targeted natural proteins or otherwise involved in natural interactions).

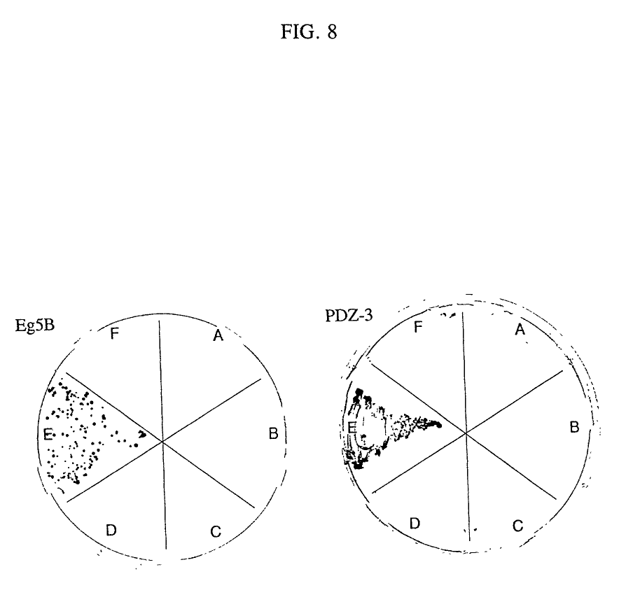

Further, it has been observed that N- and C-termini are usually spatially close to each other under physiological condition. See, e.g.,Thornton et al., 1983, Implications of N and C-terminal proximity for protein folding, J. Mol. Biol. 167:443-60 (FIG. 8). Therefore, a suitable bivalent binding domain binding simultaneously to both termini may be of greatly increased specificity and affinity compared to a univalent domain.

This invention may also recognize non-terminal peptide sequences in certain conditions. If non-terminal peptide sequences can be exposed externally, for example, in denaturing conditions, in a substantially flexible and extended conformation, then such sequences may also be accessible to engineered binding domains that are stable in these conditions. Such recognition is less preferable because denaturing conditions, even if nominally reversible, followed binding of a domain to a normally hidden peptide sequence may disrupt secondary and tertiary structures sufficiently to impair biological function. Alternatively, if a non-terminal peptide sequence is normally externally exposed, then it may also be accessible to domain binding. This is less preferable, since such a short sequence will generally have a relatively fixed conformation, which must be known in advance (or modeled or predicted in advance) and met by any engineered binding domain.

The present invention may also start with precursor binding domains targeted to small molecules, for example, binding domains for non-peptide hormones. The methods of this present invention may then engineer binding domain to other small molecules of the same general shape. Such binding domain can find use in, for example, rapid and inexpensive assays for their target small molecules, which might be environmental toxins, or drugs of abuse, or so forth.

### Preferred Precursors

A preferred precursor domain is a protein which, inter alia, has stable spatial structures and binds to one or more short peptide sequences, or to one or both terminal peptide sequences of another protein (the “native” or “initial” or “natural” targets). Preferably, the precursor's spatial structure is already known, because the rational design methods of this invention may employ the precursor's spatial structures (both bound to its target peptide and free) to great advantage. If not known, this structure may be determined by the well-known experimental means of X-ray crystallography (“XRD”) or nuclear magnetic resonance (“NMR”), or less preferably by modeling.

A precursor is preferably a naturally-occurring protein or derived from an naturally-occurring protein, such as by being one chain or a fragment of one chain of a naturally-occurring protein, or a naturally-occurring protein without its normal post-translational processing, or so forth. Where the use of the engineered domain is in a biological system, e.g., intracellularly, or in a model of a biological system, then the precursor domain may also be preferably selected for certain relevant functional properties. A precursor domain preferably plays a role in the biological function being investigated, for example, by being part of transport, signaling, synthetic or other type of protein in which it serves to bind (or release) a natural target. Therefore, a precursor is preferably selected in view of its intended use, and is preferably engineered to preserve its functions.

Finally, it is also advantageous that the precursor be capable of ready synthesis by recombinant or synthetic chemical technologies. Non-naturally-occurring protein precursors are equally usable if they satisfy the same preferences and guidelines and may be chemically synthesized.

Numerous possible precursor protein domains may be routinely obtained upon searching well-known and publically-accessible databases, for example the Protein Data Bank (“PDB”) or the Molecular Modeling Database (“MMDB”). See, e.g., Berman et al., 2000, The Protein Data Bank, Nucl. Acids Res. 28:235-242 and www.rcsb.org (web site of the Research Collaboratory for Structural Bioinformatics); Molecular Modeling Database, National Center for Biotechnology Information, Bethesda, Md. and www3.ncbi.nlm.nih.gov/Structure/. These databases generally include primary structures, spatial structures, and associated functional information for numerous proteins. These databases also make publically available computer tools for viewing and investigating the structures of the stored proteins.

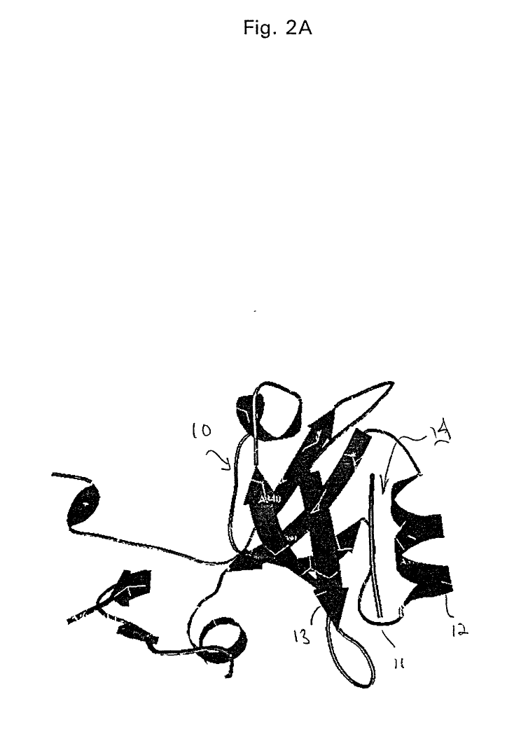

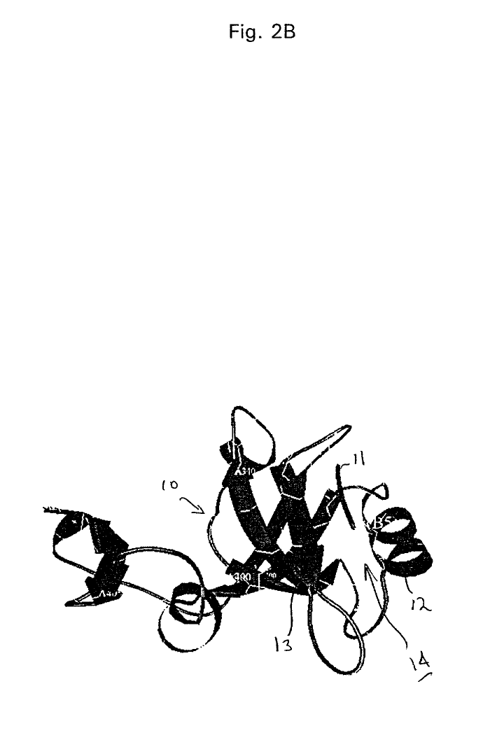

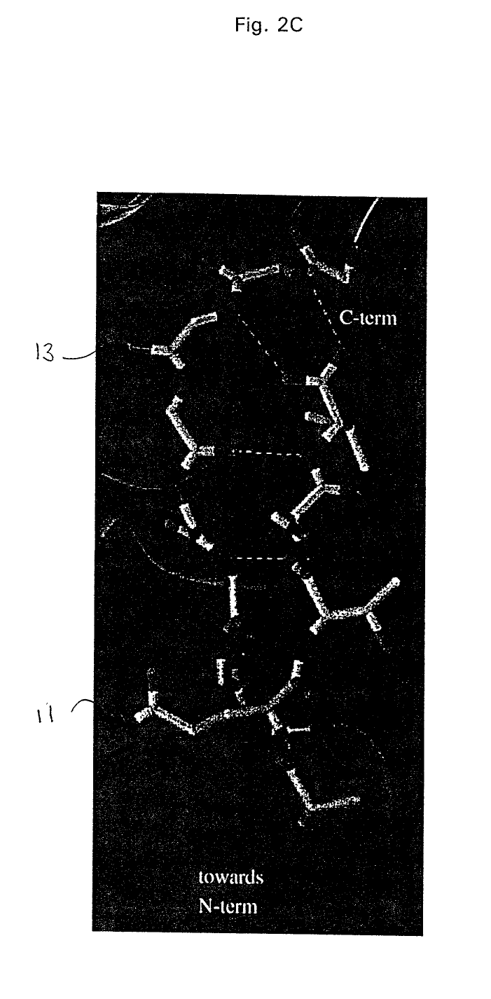

In the following, three specific exemplary precursor domains are described as illustrations of routine selections from the above protein databases. Further possible precursors are also enumerated. FIGS. 2A-C illustrate spatial structure of a first exemplary precursor domain, which is a fragment of chain A of the protein Psd-95, with a C-terminal peptide, part of the protein Cript, its natural target, bound in its binding region. See, e.g., Doyle et al., 1996, Cell 85:1067; PDB id. 1BE9. The sequence of bound C-terminal peptide part of Cript is the following:

Lys Gln Thr Ser Val (SEQ ID NO: 1)

FIG. 2A is a plan view illustrating spatial conformation 10 of this protein fragment with α-helices and β-sheets conventionally illustrated, and with bound C-terminal peptide 11. The binding region here is groove/pocket 14 bounded on two sides by α-helix 12 and β-sheet 13 of conformation 10. Side chains of α-helix 12 and β-sheet 13 residues contact and interact with the side chains of the bound peptide residues to specifically bind it in the illustrated substantially extended conformation. FIG. 2B is a perspective view of binding groove 14 which clearly demonstrates how the bound peptide fits between α-helix 12 and β-sheet 13. FIG. 2C further illustrates a detail of the interaction of the bound peptide and a portion of segment 13. Part of this interaction are the hydrogen bonds illustrated as dashed lines between amide nitrogens of the domain backbone and carboxyl oxygens of the bound C-terminus. For example, to engineer this domain to bind a different peptide sequence, one or more of the amino acids of segment 13 may need to be changed to appropriately interact with the residues of the new sequence.

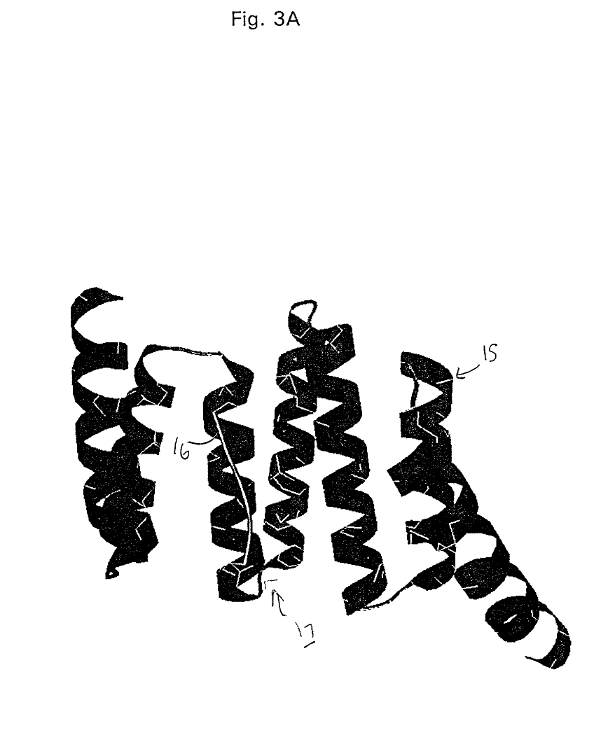

FIGS. 3A-B illustrate spatial structure of a second exemplary precursor domain, a fragment of chain A of the Tpr2A domain of Hop, with a C-terminal pentapeptide part of its natural target, the protein HSP-90, bound to its binding region. See, e.g., Scheuffler et al., 2000, Cell 101:199; PDB id. 1ELR. FIG. 3A is again a plan view of spatial conformation 15 with bound C-terminal peptide 16. Here the spatial structure is quite different from the preceding example, consisting of 7 generally parallel α-helices, certain of which forming binding groove 17. Perspective view FIG. 3B illustrates with greater clarity how binding groove 17 is substantially bounded by the four nearest α-helices. To engineer binding to a different peptide, the residues of these α-helices facing the bound peptide can be altered. Further, since these α-helices are stabilized by H-bonds between backbone atoms and since changed residues are widely spaced apart on the α-helices, substituting residues facing the binding groove can be expected to have substantially no effect on the spatial structure of this protein.

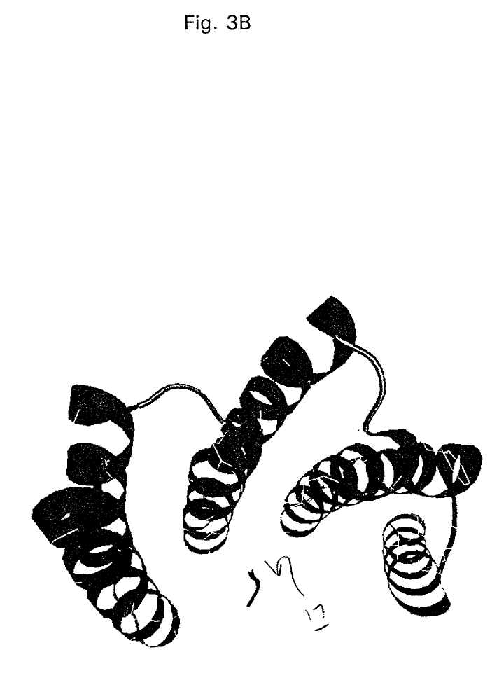

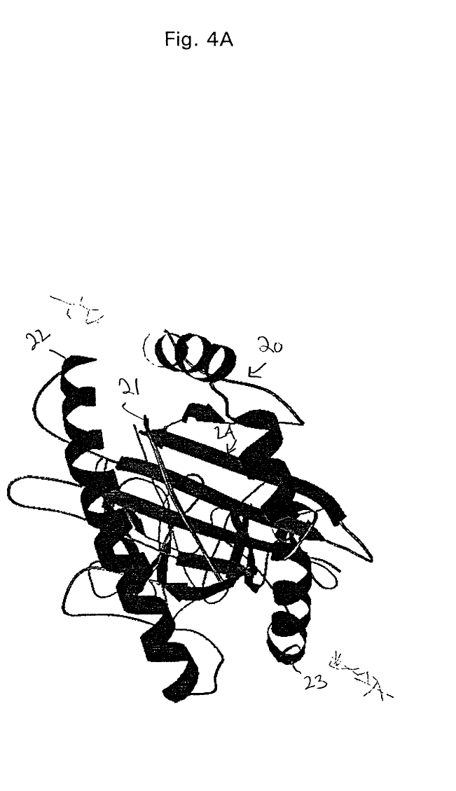

FIGS. 4A-B illustrate the spatial structures of a third exemplary precursor domain, fragments of chains H and L of Mhc class I H-2Ld, with a peptide modeling the protein Q19 bound to its binding region. See, e.g., Speir et al., 1998, Immunity 8:553; PDB id. 1LDP. FIG. 4A, another plan view, illustrates how the two chain fragments associate to form conformation 20 which defines yet another type of binding groove/pocket for bound peptide 21. This groove is laterally bounded by α-helices 22 and 23 and has a floor formed by five anti-parallel β-sheets, one of which is β-strand 24. FIG. 4B illustrates, in a partial perspective view, the binding groove bounded by (α-helices 22 and 23 and anti-parallel β-strands, such as β-strand 24. As with the previous precursors, the present precursor is a favorable engineering target because the binding groove is bounded by numerous amino acid residues that are widely spaced apart on separately stabilized secondary structures.

The precursor domains illustrated in FIGS. 2A-4B are but examples of the many precursor domains for use in this invention that can be found among the families of domains well known to bond to terminal peptide sequences of proteins, such as amino-peptidases, carboxy-peptidases, heat-shock proteins and chaperones, PDZ domains. For example, PDZ domains recognize the following amino acid consensus sequence X-Thr/Ser-X-Val, where X is any amino acid. See, e.g., Doyle et al., 1996, Cell, 65, 1067-1076. Several structures describing a complex of a PDZ domain with a bound peptide are available from the PDB, some of which are listed in Table 1. In these structures, the C-terminal carboxyl group of the peptide is strongly bound to the PDZ domain by means of hydrogen bonds.

TABLE 1
Exemplary PDZ domains

| | Protein | Experimental | |
|---|---|---|---|
| PDB id. | Description | Technique | PDB Reference |
| 1B8Q | Neuronal Nitric | NMR, 15 structures | Tochio et al., 1999 |
| | Oxide Synthase | | |
| 1BE9 | PSD-95, Cript | X-ray, 1,82 A | Doyle et al., 1996 |
| 1KWA | HCASK/LIN-2 | X-ray, 1.93 A | Doyle et al., 1996 |
| 2PDZ | Syntrophin | NMR, 15 structures | Schultz et al., 1998 |

Specific binding to the C-terminal region of a protein is also observed with other protein binding domains, where binding to the terminal carboxy group is essential. Table 2 lists exemplary domains of this type.

TABLE 2
Exemplary C-Terminal Binding Domains

| | Protein | Experimental | |
|---|---|---|---|
| PDB id. | Description | Technique | PDB Reference |
| 1ELR | TPR2A-Domain of HOP | X-ray, 1.9 A | Scheufler et al., 2000 |
| 1ELW | TPR1-Domain of HOP | X-ray, 1,6 A | Scheufler et al., 2000 |

Specific binding to the N-terminal region of a protein is observed with still other protein binding domain where binding to the terminal amino group is essential. Table 3 lists exemplary domains of this type.

TABLE 3
Exemplary N-Terminal Binding Domains

| | Protein | Experimental | |
|---|---|---|---|
| PDB code | Description | Technique | Reference |
| 1A16 | Aminopeptidase p from e. coli | X-ray 2.3 | Wilce et al |
| 1B59 | Human aminopeptidase-2 | X-ray 1.8 Å | Liu et al. |
| 1B11 | Leu aminopeptidase | X-ray 2.4 Å | Kim et al. |
| 1C21 | E. coli Met aminopeptidase | X-ray 1.8 Å | Lowther et al. |

Alternatively, specific binding to a terminal region of a protein may be obtained using precursors where binding to the amine or carboxyl groups is not essential. Table 4 lists exemplary domains of this type.

TABLE 4
Exemplary N- or C-Termini Binding Domains

| PDB | Protein | Experimental | |
|---|---|---|---|
| code | Description | Technique | Reference |
| 1AQD | HLA-DR1 Class II | X-ray, 2.45 A | Murthy et al., |
| | Histocompatibility/HLA-A2 | | 1997 |
| 1LDP | Complex MHCI/Peptide | X-ray, 3.1 A | Speir et al., 2000 |

Many further possible and preferred precursor domains are readily available in the public protein databases. The previous specific and general examples of precursor domains usable in this invention are not to be taken as limiting. Any domain satisfying the above preferences and guidelines can be used in the present invention.

### Select Candidate Domains

From a selected precursor domain, or from candidate domains selected in a previous iteration (collectively designated “precursor candidates”), new alternative candidate domains (collectively designated as a “space of alternative domains”) are selected by rational design/selection methods. This space is then screened, such as by the preferable CAMD techniques. The alternative-domain space is preferably selected it is likely to contain substantially all of improved candidate binding domains that can be engineered from the precursor candidates. In a preferred embodiment, candidates in this space are select by, first, choosing residues that are likely to be involved in contacting or interacting with the new target peptide sequence, and, second, by choosing possible amino acid substitutions or mutations at those chosen residues that are expected to positively contribute to the binding or stability of the new target-candidate complex.

These choices are made according to rational selection techniques, for example, by rules known in the art, by rules supplemented by computer calculations, or entirely by computer-based techniques. In one basic embodiment, likely interacting residues are chosen by visual inspection and manipulation of a computer-generated model (as is well known in the art) of a precursor candidate bound to the target. Those residues of the precursor candidate that form a first structural layer adjacent to or around the bound peptide, for example, by having side chains in proximity to the side chains of the bound peptide, can be visually determined from this model and selected for mutation or substitution. In further embodiments, this selection may be made by computer assisted. For example, side chains of the precursor candidate residues and of target peptide residues may be modeled geometrically, and those residues contacting target peptide residues chosen for substitution. One geometric model models amino acid side chains as cones having an axis along the C_{α}-C_{β} vector and an appropriate width, with the C_{α}-C_{β} vector inclination being determined from a rotamer library (see subsequently). A more elaborate model, instead of looking for geometric contacts, approximates the energy of interaction of precursor candidate and target peptide backbone side chains, for example by a known energy function including van der Waals terms, H-bonding terms, electrostatic terms, and solvation terms such as is used in molecular dynamics. All residues of the precursor candidate for which the interaction action energy with the target peptide is sufficiently high, for example, being more than approximately 20% of their interaction energy with neighbors, may be chosen for substitution.

In certain situations it may be necessary to chose additional residues that, although not adjacent or around the bound initial peptide, are likely to be affected by the new target. For example, during initial choice, size differences between the initial peptide and the new target peptide may suggest that additional changes to more remote residues may be advantageous to accommodate needs for new space or to occupy newly empty space. Also, more remote amino acids may need to be substituted to create hydrophobic surfaces or pockets for hydrophobic target peptide residues. Importantly, if an initial choice led to candidate domains that did not adequately bind to the new target, then further visual inspection or quantitative analysis of the most promising of the candidate of the prior iteration may suggest additional more remote residues which should be included in defining the space of alternative domains.

Next, for each residue selected for substitution, one or more new amino acids are chosen, which are expected to positively influence binding of the new target. A random mutagenesis which substitutes all 19 other amino acids at each selected interacting residue is much less preferable. Preferably, for each selected residue, a subset of the possible amino acids is carefully selected by rational techniques that is expected a priori suitable, for example, both in terms of their interaction with the new target when bound and also with other domain residues adjacent to a binding site. In one embodiment, the rational techniques simply employ well-known classifications of new amino acids to be “like” amino acids being replaced in the candidate, or to interact favorably with amino acids in the new targets. The following table presents one such classification (in which an amino acid may be multiply classified).

TABLE 5
Exemplary Amino Acid Classification

| Amino Acid Classification | Amino Acids |
|---|---|
| Hydrophobic | Ala, Val, Ile, Leu, Phe, Met, |
| | Trp |
| Hydrophilic | Ser, Thr, Asn, Gln, Asp, Glu, |
| | His, Lys, Arg, Tyr |
| Basic | Arg, Lys, His |
| Acidic | Asp, Glu |
| H-bond participants | His, Ser, Thr, Asn, Gln, Tyr, |
| | Trp |
| Aromatic | Tyr, Phe, Trp |
| Small | Ala, Gly |
| Bulky | Trp, His, Phe, Tyr |
| Secondary structure “breakers” | Gly, Pro |
| β-branched chain | Ile, Val, Thr |
| Metal chelators | Cys, Met, His |

In view of such a classification, an exemplary amino acid substitution rule chooses basic amino acids for substitution for residues contacting (or interacting) with acidic residues of the target peptide, and vice versa. Another rule may substitute hydrophobic amino acids at residues contacting hydrophobic target peptide residues. At target peptide residues where an H-bond with a side chain is possible, amino acids capable of H-bonding (either donor or acceptor) should be chosen (for example, polar, basic, or acidic amino acids). Amino acid substitutions that clearly lead to overlap or other negative steric hindrances should be avoided a priori. For example, where space available in a binding region is limited, perhaps because the new target peptide has a bulky residue, substitutions in the precursor should avoid bulky amino acids. Thus, Trp, Phe, and possibly Leu, should not be substituted where hydrophobic contacts are needed in limited available space, and vice versa.

More elaborate rational techniques may quantitatively determine spatial and steric considerations by evaluating precursor and target residue side-chain geometry using models similar to those described above for selecting precursor residues for substitution in the first place. Still further rules derive from more quantitative consideration of the interaction of a changed binding domain with its new target. To minimize negative solvation effects, hydrophobic residues should not be solvent exposed. On the other hand, at solvent exposed sites, polar or H-bond capable residues should be favored for substitution. The degree of solvent exposure can be simply ranked knowing the environment and orientation of the selected residue, alternatively supplemented by general ranking of the size and shape of the considered amino acid, for example quantitated as a solvent exposed surface area.

Finally, in the case of proteins with post-translational modifications at the N- or C-termini, i.e., formylation of the N-terminal Met, the rules used for selecting alternative domains may be adapted in order to take the modification into account. For example, the interaction properties and the size of the modification may change the amino acids advantageously selected for a particular residue.

Further still more elaborate embodiments of these rational techniques may, instead of the above rules, systematically choose amino acids based on more accurate models of side-chain interactions. For example, amino acids may be ranked in order of increasing suitability as a replacement at a selected residue with an efficient protein model, so that the subsequent step of screening the space of alternative domains can proceed from the most suitable amino acid mutations to the less suitable. Such an efficient ranking model may, for example, be limited to fixed precursor domain and target backbone geometries, amino acid models limited to simple geometries and net interaction (that is, for example, net electrostatic, van der Waals, hydrophobic interactions), and side chain orientations limited to those in a simple rotamer library.

Further, the steps of defining a space of alternative domains and then exploring domains in the defined space may be combined so that the alternative domains are defined as they are explored. That is, these actions may be linked in a single “define and explore” loop.

The example illustrated in FIGS. 2A-C illustrates selection of precursor-domain residues for substitution and then selection of amino acids to substitute. By visual inspection of the conformations at higher resolution than illustrated, it was determined that the residues forming the binding groove and interacting with the initial peptide target are L323, F325, N326, I327, I328, E331, H372, E373, A376, L379 and K380 (with residue numbering relative to the protein chain of Psd-95 only a fraction of which appears in PDB id. 1BE9). The three additional residues, I338, L342 and I359, were further determined to possibly affect binding of certain new target peptides. Table 6 below summarizes the binding region interactions in this example. (Residue numbering of the initial target peptide begins at 5.)

TABLE 6
Interacting Residues of an Exemplary PDZ Domain

| Residue | Secondary | Residue | Contacting |
|---|---|---|---|
| Position | Structure | Buried/ | Residue(s) of |
| in Domain | at Residue | Exposed | Bound Peptide |
| 323 | β-strand | Buried | 9 |
| 325 | β-strand | Buried | 7, 9 |
| 326 | β-strand | Exposed | 6, 8 |
| 327 | β-strand | Buried | 5, 7, 9 |
| 328 | β-strand | Buried | 6, 8 |
| 331 | Loop | Exposed | 5 |
| 372 | α-helix | Exposed | 5, 6, 7 |
| 373 | α-helix | Exposed | 5, 7 |
| 376 | α-helix | Exposed | 5, 7, 8, 9 |
| 379 | α-helix | Buried | 7, 9 |
| 380 | α-helix | Exposed | 7, 8, 9 |

Guided by this table, amino acids for substitution at specific residues may be selected in accordance with above-described rules and preferences to favorably interact with a new target peptide sequence. This table indicates the buried/exposed status of the interacting residues, and preferably hydrophobic amino acids should be substituted at buried sites and polar or H-bonding amino acids should be selected for exposed sites. For example, if the new target has a terminal Leu in place of Lys of the initially target, then hydrophobic replacement(s) should be selected for the residues in the buried binding domain contacting residue 5 of the bound peptide, namely I327; the remaining contacting residues, E331, H372, E373 and A376, are exposed and should preferably not be hydrophobic.

### Screening Candidate Domains

The screening step (step 4 in FIG. 1) screens or explores a space of alternative candidate domains provided by a previous rational selection step from the original precursor domain, or from a previously screened space of precursor candidate domains provided by a previous iteration of the “select and test” process of this invention. The screening methods employed in a particular iteration are preferably selected in view of the method of rational selection employed by the immediately preceding selection step.

Briefly, this invention contemplates at least one screening (or exploration) step employing rational screening techniques, that is techniques that are based on a priori physical or chemical principles, or knowledge of those of skill in the art, or rules codifying such knowledge (the latter two are called “empirical” rational methods herein). After defining a space of precursor candidate domains, either from the initial precursor of from previous candidate domains, the present invention screens (or “explores”) this defined space to find one or a few candidate domains that are predicted to bind to the new target with improved specificity and affinity (“improved” candidates). The invention further contemplates a final step that screens a reduced space of alternative candidates by employing laboratory techniques to determine actual experimental affinities, or to check that the candidates serve for their intended functions, or so forth. However, it is preferable that laboratory techniques are only employed when prior rational techniques have narrowed the space of candidate binding domains to a small number of members, for example, less than 10,000, or more preferably 1,000, or even 100 or fewer members. Thereby, the present invention is more efficient than prior techniques relying on random mutagenesis, which provides enormous number of candidate domains for exploration. More efficient and rapid rational techniques replace more costly and slower laboratory methods. Moreover, if the rational selection and screening employed are of proven accuracy, then laboratory screening may be dispensed with entirely, resulting in even greater relative efficiencies.

The present sub-section describes exemplary rational screening techniques, while the subsequent section describes laboratory techniques (reserved for a final focused screening) for exploration of alternative candidates. Preferred empirical rational techniques are based on empirical rules that summarize experience in the art relating to actual examples of binding of peptide ligands to polypeptide domains. Experimental studies concerning the binding of various ligands to members of protein-binding-domain classes have been increasing in the art. This experimental information may be summarized as formal or informal rules, and applied to select precursors from the studied classes and to screen candidate domains derived from the studied classes for binding to new peptide ligands. These preferred rational methods are more rapid but less accurate than computer-assisted molecular design methods, and may therefore be advantageously employed in early iterations of the methods of this invention. Examples of empirical rules can be found in, e.g., Brannnetti et al., 2000, SH3-SPOT: an algorithm to predict preferred ligands to different members of the SH3 gene family, J. Mol. Biol. 298(2): 313-28; Baxter et al., 1998, Flexible docking using Tabu search and an empirical estimate of binding affinity, Proteins 33(3): 367-82; Bohm, 1998, Prediction of binding constants of protein ligands: a fast method for the prioritization of hits obtained from de novo design or 3D database search programs, J. Comput. Aided Mol. Des. 12(4): 309-23; Eldridge et al., 1997, Empirical scoring functions: I. The development of a fast empirical scoring function to estimate the binding affinity of ligands in receptor complexes, J. Comput. Aided Mol. Des. 11 (5): 425-45; Kauvar et al. 1995, Predicting ligand binding to proteins by affinity fingerprinting, Chem. Biol. 2(2): 107-18; Murray et al., 1998, Empirical scoring functions. II. The testing of an empirical scoring function for the prediction of ligand-receptor binding affinities and the use of Bayesian regression to improve the quality of the model, J. Comput. Aided Mol. Des. 12(5): 503-19. The following references describes uses of empirical rules and chemical knowledge common in the art for modifying ligand binding specificity and affinity, e.g., DelValle et al., 1995, Construction of a novel bifunctional biogenic amine receptor by two point mutations of the H2-histamine receptor, Mol Med 1(3): 280-6; Riechmann et al., 1992, Improving the antigen affinity of an antibody Fv-fragment by protein design, J. Mol. Biol. 224(4): 913-8.

Other exemplary rational techniques are based on methods of homology modeling known in the art. Homology modeling methods generally approximate the structure or properties of a candidate polypeptide domain by the structures of homologous proteins and protein fragments found in protein structure databases. Homologous proteins preferably have statistically-significant amino-acid-sequence similarities, and optionally similar biological derivations. Approximate structure for an alternative candidate may be obtained by homology modeling, and then used to estimate the binding of the new target peptide, by, for example, use of docking tools that estimate new target binding by searching for a lowest energy alignment of the new target in the approximate structure determined for the binding pocket of the alternative candidate. Candidates with the best estimated binding energies are selected for subsequent processing. Conversely, as described below, homology modeling may be used to select new candidates. For example, proteins found by modeling to be homologous to the certain structural alternatives may provide sequence substitutions defining improved candidate domains. Homology has other application in the present invention. For example, consensus binding sequences in protein structure databases that bind to short peptide sequence fragments (for example, of 1-4 amino acids) may be combined in “chimeras” that are likely to be binding candidates for longer target peptide sequences. Homology modeling may also be used to improve the stability of newly found candidate (perhaps even one with adequate binding). Tools for homology modeling include WHATIF (Vriend, 1990, Mol. Graph. 8:52). Improving candidate stability by sequence comparison or empirical approximation are described in, e.g., Wang et al., 2000, Stabilization of GroEL mini-chaperones by core and surface mutations, J Mol Biol 298(5): 917-26; and Lopez-Hernandez et al., 1995, Empirical Correlation for the Replacement of Ala by Gly: Importance of amino acid secondary intrinsic propensities, PROTEINS: Struct. and Function 22: 340-349. Methods for producing chimeric proteins with synergistic target-binding properties are described in, e.g., Campbell et al., 1997, Chimeric proteins can exceed the sum of their parts: implications for evolution and protein design, Nat. Biotechnol. 15(5): 439-43; Guerrini et al., 1998, Rational design of dynorphin A analogues with delta-receptor selectivity and antagonism for delta- and kappa-receptors, Bioorg. Med. Chem. 6(1): 57-62; Shimoji et al., 1998, Design of a novel P450: a functional bacterial-human cytochrome P450 chimera, Biochem. 37(25): 8848-52.

Further, empirical rational methods used in this invention may be improved as a consequence of the operation of the invention itself. Simply, operation of the present invention engineers polypeptides with new binding specificities having known affinities from precursor polypeptides with known binding specificities and affinities. Comparison of engineered polypeptides with their precursors may then directly yield information on the influence of the amino acid substitutions on binding specificity and affinity. This information may then be fed back to improve the accuracy of empirical rational methods originally used in its derivation. Therefore, in preferred embodiments, the cumulative results of operation of this invention are stored for such comparison and improvement, and succeeding applications of this invention make use of the thereby improved empirical rational methods.

It is preferred that at least one step of exploring/screening employ computer-assisted molecular design (“CAMD”) methods, which are taken herein to be methods which are based on physical and chemical principles advantageously approximated to achieve decreased computation time but adequate accuracy in determining small changes to a largely unperturbed polypeptide. A wide range of CAMD methods may be effectively used in the present invention. Ideally, a CAMD method would provide substantially precise binding free energies of the new target to an alternative candidate domain. Step 4 then selects those candidate domains having binding free energies sufficient for acceptable binding affinities. Since general methods for computing free energies are not yet available for general protein structures, or if available, require great computation resources, it is preferable to choose approximate CAMD which, after reasonable computation times, provide a scoring function (“free energy scoring function”) that is substantially related to, or approximates, the actual free energy of binding, or at least substantially preserves the relative ranking of the alternative domains according to the free energy of binding (that is substantially monotonically related to the actual free energy). Then, selected candidate domains are those alternative domains that have reach some, possibly empirically-determined, level of the scoring function. In practice, the accuracy of the approximations underlying a particular CAMD method and its resulting free energy scoring function can be determined by comparing the results of the predicted scoring with the actual binding affinity (in view of the intended use of the engineered binding domain). If a particular method is proven to be sufficiently accurate, then experimental confirmation of actual binding can be dispensed with, resulting in considerable efficiencies. Since accuracy of a particular CAMD method may generally be related to the computational effort required to practice the method, this invention contemplates a tradeoff between a particular CAMD method and prior more approximate rational selection and screening steps and a subsequent laboratory confirmation or screening. Different tradeoff points may be appropriate in different applications or embodiments of the present invention.

Preferred CAMD methods rely on two basic structural approximations that decrease computational requirements: first, an “inverse folding” approach for modeling the backbones of the precursor domain and target peptide; and second, a rotamer library approach for modeling side-chain conformations. Inverse folding techniques (as this term is used herein) consider protein backbones (defined by, for example, the phi and psi angles that specify the orientation of the C_{α} and (C═O)NH groups), both the backbone of the candidate domain and that of the new target, as substantially unchanged by the substitution of new residues, or as substantially unchanged throughout the CAMD methods. Therefore, the free energy scoring function does not depend on the peptide backbones. Small backbone displacements as discussed subsequently are consistent with such substantially fixed backbones.

Next, all precursor-domain residues are classified as either “fixed” or “non-fixed”. A fixed residue has a fixed side chain conformation. All residues are fixed, with the exceptions of new target residues, of mutated residues in the candidate domain, and of residues strongly interacting with one of the non-fixed residues, e.g. by being a nearest neighbor of one of the non-fixed residues, or if not a nearest neighbor, by being expected to have significant interaction with a changed residue. This interaction can be evaluated by a molecular energy function used (described below). Side chain conformations of the non-fixed residues are allowed to vary, but assuming only the configuration of rotamers in an appropriate rotamer library. See, e.g., Janin & Wodak, 1978, J. Mol. Biol. 125:357-386; Ponder & Richards, 1987, J. Mol. Biol. 193:775-791.

A rotamer library is a compilation, for each amino acid, of statistically common conformations assumed by its acid side chain in a wide variety of proteins, for example those protein structures from those deposited in the PDB. Side chain conformations for each amino acid may be by dihedral angles (χ₁, χ₂, χ₃, χ₄) between adjacent the side chain carbon atoms (the number of dihedral angles is appropriate to the particular side chain). These dihedral angles can be compiled independently for each amino acid (backbone-independent rotamer library) or may be conditional on the backbone configuration (backbone-dependent rotamer library). It is preferably to also explore small perturbations to the rotamers from the library in order to more accurately model side-chain behavior. Generally, given sufficient computational resources, the more extensive backbone-dependent rotamer libraries are preferred.

Next, using these two structural approximations, the thermodynamic free energy of a new target bound to an alternative domain is generally approximated as a sum of the enthalpic and entropic components (the latter multiplied by an “effective” temperature) due to (i) the precursor backbone and the non-mutated residues (“the template”), (ii) the mutated residues interacting with the template, and (iii) pair-wise interactions among the mutated residues. Each component is evaluated against a suitable reference state, for example the precursor domain bound to its initial target. In this case, the resulting free energy function scores the affinity of the alternative domain relative to the affinity of the precursor domain. Here, the template terms become small because the template is approximated as substantially unchanged. A more complex reference is a denatured state, in which case the resulting free energy provides an approximate score of the absolute affinity of the new target and alternative domain.

Each enthalpic component, generally the molecular energy, is preferably evaluated by including the well-known non-bonded interactions: van der Waals energies, hydrogen bond energies, and electrostatic energies. These interactions may be evaluated by any of the well known molecular energy potentials: for example, AMBER, CHARMM, ECEPP2, MM2, CVFF, all of which are commercially or freely available. ECEPP/2 is preferred.

Each entropic component is generally divided into a solvation term, a template term, and a side chain term. The solvation term is estimated by multiplying for each atom an empirical solvation energy per solvent-accessible surface area (“SAS”) and a change in the SAS. The changes in SAS in the template atoms are due to effects of the changed residues; further the changed residues have a changed SAS with respect to the original residues; finally pairs of changed residues change each other's SAS. Next, the template and side-chain entropies are essentially rotational entropies. Fixed template entropy is evaluated with respect to an “ensemble” representing accessible conformations (in terms of available ψ-φ dihedral angle space) for the particular sequence, this ensemble being derived by assuming configurations of non-homologous proteins in a protein library are all equi-probable. This entropy preferably takes into account changes in the available dihedral angle space between the precursor and the alternative domain due to the residue substitutions. Similarly, the entropy increase represented by a particular side chain conformation is evaluated with respect to an “ensemble” representing accessible side-chain conformations (in terms of available χᵢ dihedral angle space) for the particular sequence, this ensemble being derived from rotamer libraries providing a statistical distribution of side-chain conformations (either unconditional and conditional on the identity of neighboring residues).

Using such a suitably approximated free-energy scoring function, the inverse-folding method then determines scores for alternative domains (“alternative-domain score”) by searching among the side-chain conformations of the non-fixed residues as allowed by the structural approximations for conformations that minimize the approximated free energy. Many techniques may be used for this search. Simple direct techniques include the steepest-descent or the conjugate-gradient methods. More complex techniques may include stimulated annealing, in which the effective temperature is gradually lowered during the search. Also certain algorithmic techniques known from computer science may by advantageously applied. See e.g., Horowitz et al., 1978, Fundamentals of Computer Algorithms, Computer Science Press. For example, branch-and-bound methods may be used to avoid searching those regions of the alternative domain space certain to have no useful structures. Alternatively, certain approximate search techniques, such as a greedy method, may be sufficient. Similar search techniques, especially branch-and-bound, may also be applied to explore the space of alternative domains.

In an alternative embodiment, where acceptable candidate domains are difficult to find using the above approximations, it may be advantageous in further iterations of the “select and test” process of this invention to allow selected perturbations in precursor domain structure. One such perturbation includes small backbone displacements (i.e., less than or approximately equal to 1-1.5×10⁻⁸ cm), either throughout the entire precursor domain backbone, or limited (or somewhat larger) in the backbone of the new target and the backbone of the precursor domain near interacting residues, e.g. withing 4 residues of the interacting residues. These perturbations may be made by allowing alterations in the phi-psi angles of certain backbone residues, such as those described above, during minimization of the free-energy scoring function. The phi-psi angles of altered residues should be coupled in well known manners so that backbone integrity is preserved. Here, the search to find the minimum approximated free energy would be expanded to include such backbone perturbations which change the enthalpic terms since atomic distances change, and preferably also the entropic terms since allowed perturbation space may differ for the alternative and precursor domains.

A further perturbation, as part of CAMD screening or as part of a subsequent rational selection step, is candidate domain remodeling by inserting, deleting, or substituting short (for example, between and residues) peptide fragments (as well as the single residues previously described) may be advantageous. Domain remodeling of a selected domain fragment, perhaps defining part of a domain binding groove or a loop linking domains defining secondary structures, can make use of fully or partially homologous fragments found in protein structure databases. For example, the relative three dimensional positions of the C_{α}'s of the selected fragment (equivalently the C_{α}-C_{α} distance and orientation) can be used to search a protein structure database for fragments of stored structure having relative positions that are the same within 1×10⁻⁸ (or 2×10⁻⁸) cm. Retrieved fragment can then be substituted or inserted at the position of the selected fragment, and the “mutated” domain then used to engineer further candidate domains. Such remodeling may be carried out with existing modeling tools, such as the WHATIF tool. See, e.g., Vriend, 1990, Mol. Graph. 8:52. This process may also be applied to domain fragments longer than 20 residues, optionally up to 25 or 50 residues. Such remodeling makes changes to all free energy components as is readily apparent from the above descriptions.

Allowing these additional perturbations may increase the computation time. The present invention may tradeoff such increased computation where the candidate domain space to search is reasonably restricted. Such costs may be acceptable in order to find suitable or improved candidate domains by CAMD techniques.

A preferred inverse-folding method incorporating the above preferences and guidelines is disclosed in U.S. patent application Ser. No. 09/387,741, filed Aug. 31, 1999, entitled “Computer-based method for macromolecular engineering and design”, and by one or more of the inventors of the present application. This application is hereby incorporated herein by reference for all purposes; the method described therein is referred to as “Perla”. Perla as preferably practiced includes both selection of candidate domains and screening of the selected candidates for binding to the new target. By combining these steps, Perla achieves algorithmic efficiencies not otherwise possible. Thus, Perla identifies amino acid sequences capable of folding into a desired peptide/protein three-dimensional structure and can be used to rational engineer/redesign amino acid sequences that fit optimally into peptide/protein and protein/protein binding interfaces. The computer program evaluates the ability of an amino acid sequence to fit a specified three-dimensional structure through a complex scoring function based on an all-atom molecular mechanics force field (van der Waals, electrostatic and hydrogen bonding) and a combination of statistical terms (entropy and solvation). This scoring function is determined with respect to a reference state that simulates denatured proteins. Structural modeling is performed generally in an inverse folding approach: a specified backbone structure is decorated with amino acid side chains taken from a rotamer library. Local energy optimization is achieved via the generation of sub-rotamers. Combinatorial problems in sequence and side chain conformation spaces are solved using dead-end elimination and mean field theory, respectively. Perla has been successfully applied in various fields of protein design.

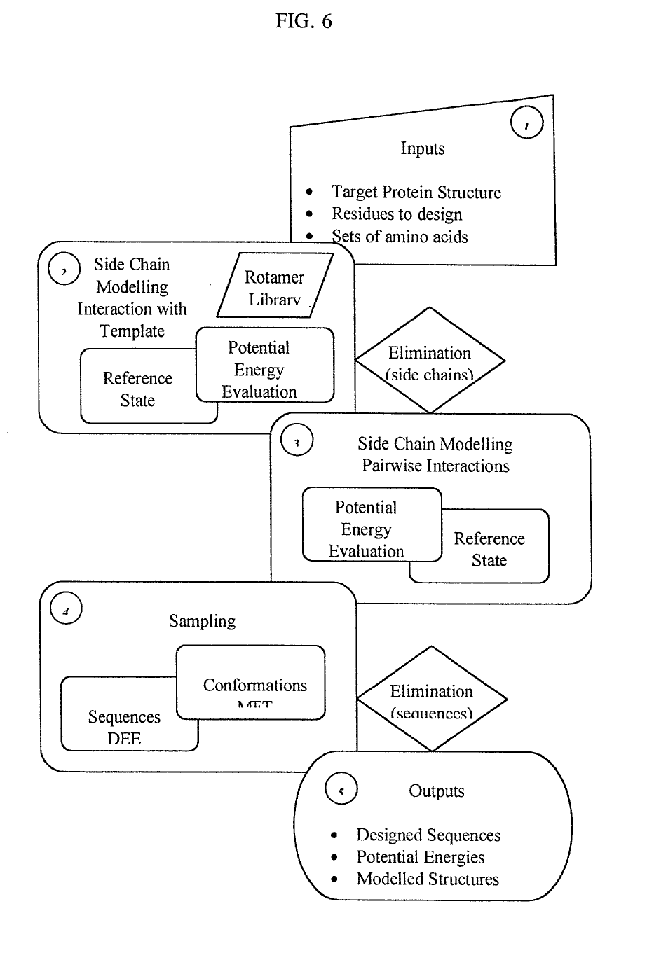

FIG. 6 illustrates Perla in detail. Side chains that correspond to the list of amino acids to model (variable and flexible residues) are obtained from a rotamer library and their interaction with the template or fixed protein conformation is computed. Van der Waals, electrostatic and hydrogen bonding are summed and optimized, and backbone entropy costs are added. Side chain conformers that are not compatible with the template structure are eliminated. Pairs of rotamers are then considered to evaluate the side chain-side chain component of the scoring function. Again, van der Waals, electrostatic and hydrogen bonding are considered and optimized. A pair-wise solvation contribution is added. No elimination is performed, since the identification of an energetically disfavored pair does not imply that the participating side chains are incompatible with the target protein fold.

The optimization of van der Waals, electrostatic and hydrogen bonding interaction energies is done by averaging the interaction energies measured for an ensemble of side chain conformers. This ensemble includes “sub-rotamers” which are generated by small rotations of the rotamer conformations taken from the rotamer library. Two rotation schemes are tried in different runs of computation: only rotations around the alpha-beta (corresponding to the χ1 dihedral angle) and beta-gamma (corresponding to the χ2 dihedral angle) bonds are considered, all rotations are by degrees, and either one or two steps are performed (in either direction). A first design with Perla is thus conducted creating for each side chain rotamer an ensemble of 3 (Cys, Ser, Thr, Val) or 9 (Arg, Asn, Asp Gln, Glu, His, Ile, Leu, Lys, Met, Phe, Trp, Tyr) sub-rotamers (for each side chain rotamer conformation) and a second design is done with ensembles of 5 or 25 sub-rotamers.

The different sequence variants modeled by Perla are evaluated in terms of van der Waals, electrostatic and hydrogen bonding interactions, plus the change in entropy and solvation upon folding the protein. Molecular mechanics energy terms (van der Waals, electrostatic and hydrogen bonding) are measured using the parameterization of the ECEPP/2 energy force field (Nemethy et al.,(1983). J. Phys. Chem. 87, 1883-1887). Entropy and solvation are statistical terms, the parameterization of which was obtained by analyzing the protein structure database. All these terms are weighed by a factor 0.5, except for the solvation energy (factor1).

Since the lower the energy the better the sequence variant, Perla searches for sequences with minimal energy. To reduce the number of sequences to sample, and eventually to find that which has the optimal sequence-to-structure relationship (lower score), the dead-end elimination (“DEE”) (Desmet, et al., (1992) Nature, 356, 539-542) is preferably used to mark and discard the amino acid that cannot belong to the minimum of energy of the sequence space. Alternatively standard branch and bound methods, as well known in the art, may be used. See, e.g., Gordon et al., 1999, Structure, Folding, and Design vol. 7:1089-1098. Then, for all remaining sequences, the mean field theory (“MFT”) (Koehl,. & Delarue, (1994) J Mol Biol 239, 249-275), or other optimization method, enables the estimation of weights for all side chain rotamers. Those weights are used to compute the score of each sequence. Sequences that do not score well are rejected, while for others, the solvation term is re-evaluated. Some sequences might then be eliminated if they have poor solvation.

The output of Perla is a set of rank ordered sequences, with a description of the energy terms that participate to the scoring function used by Perla along PDB-formatted coordinate files of the modeled structures. Selection of the sequence variants to be used in further iterations or to be assayed experimentally is done choosing from the best sequence candidates, usually after careful visual inspection of the various modeled structures. Moreover, depending on available computer resources, Perla can take from hours to as little as 10 minutes (depending on the number of residues mutated and the number of rotamers per new amino acid).

This invention is to be understood as not limited to Perla or to CAMD methods satisfying the above described preferences and guidelines, at least the following alternatives are apparent. Especially in connection with improved or heuristic search algorithms, generation of alternative domains can be coupled with their exploration, instead of generating the space of alternatives in advance of its exploration. Where efficient screening for actual binding is possible, e.g., by using laboratory robots in connection with array-formatted synthesis arrangements, less accurate but more rapid CAMD techniques can be used, generating more candidate domains. Further alternatives readily apparent to one of skill in the art in view of this description are within the scope of the present invention.

Finally, output of the CAMD methods can take several forms. In one embodiment, the alternative domains can simply be accepted or rejected as candidates. It is preferable for the output to include a ranked list of alternatives with an indication of the final minimum score so that an improved selection of candidates is possible. For example, molecular models of the few best ranked alternatives can be examined, perhaps manually perhaps by comparison with protein database, for reasonableness as part of candidate selection. However presented, the selected candidate domains are then assayed for actual binding. As noted, this assay may be optional if the CAMD results are sufficiently accurate for the intended purposes.

### Screening Methods

Having one or more candidate binding domains engineered for binding to a new target, laboratory screening methods may be employed to assay the actual binding affinity and specificity. A number of screening methods may be, for example, methods depending on reconstitution of a transcriptional activator, such as yeast two hybrid systems, and methods depending on directly observing binding, such as affinity chromatography and biosensor analysis, and the like, and phage display. Although, the assay methods described herein are the methods generally preferred, other methods known in the art that assay binding specificity and affinity can be employed in the assays of this invention.

### Reconstitution Methods

These are methods in which binding of a first test protein to a second test protein reconstitutes a transcriptional activator which causes expression of a reporter. For application in the present invention, one of these test proteins includes a candidate binding domain, and the other test protein includes the new target. Where the methods permit screening libraries of first or second test proteins (or of both) for binding, then one library may include all the candidate binding domains engineered to a new target so that they may be all assayed at once and the acceptable candidates selected. Alternatively, one library may include a plurality of other targets (perhaps combinatorially determined) along with the selected target so that specificity of binding may be assayed. Preferably, when both types of libraries can be simultaneously screened, then both assays can be efficiently performed at once.

### Protein-Pair Reconstitution Methods

The following reconstitution methods are primarily adapted to screen a single pair of proteins for interaction.

A first example of a reconstitution method is the now well known yeast two-hybrid system in the yeast Saccharomyces cerevisiae (Fields and Song, 1989, Nature 340:245-246; U.S. Pat. No. 5,283,173 by Fields and Song). This assay detects an interaction between two known proteins by utilizing the reconstitution of a transcriptional activator like GAL4 (Johnston, 1987, Microbiol. Rev. 51:458-476) through the interaction of two protein domains that have been fused to the two functional units of the transcriptional activator: the DNA-binding domain and the activation domain. This is possible due to the bipartite nature of certain transcription factors like GAL4. Being characterized as bipartite signifies that the DNA-binding and activation functions reside in separate domains and can function in trans (Keegan et al., 1986, Science 231:699-704). The reconstitution of the transcriptional activator is monitored by the activation of a reporter gene like the lacZ gene that is under the influence of a promoter that contains a binding site (Upstream Activating Sequence or UAS) for the DNA-binding domain of the transcriptional activator.

To use the standard yeast two-hybrid assay in this invention, one of the domains of the transcriptional activator is fused to the candidate engineered binding domain and the other domain is fused to the new target, either the peptide or to the protein having the peptide for a C-terminus (with appropriate vectors, N-terminal peptides can also be assayed). This standard assay may also identify interacting proteins from a population that would bind to a known protein (Durfee et al., 1993, Genes Dev. 7:555-569; Gyuris et al., 1993, Cell 75:791-803; Harper et al., 1993, Cell 75:805-816; Vojtek et al., 1993, Cell 74:205-214), and in this invention, the population may be either several engineered candidate binding domains or a population of potential targets.

There are many versions and improvements to the standard two-hybrid assay that can be usefully applied. One version is the “Interaction-Trap system” devised by Brent and colleagues (Gyuris et al., 1993, Cell 75:791-803), which is similar to the Two-Hybrid system except that it uses both a LEU2 reporter gene and a lacZ reporter gene. Thus protein-protein interactions leading to the reconstitution of the transcriptional activator also allow cells to grow in media lacking leucine and enable them to express β-galactosidase. The DNA-binding domain used in this system is the LexA DNA-binding domain, while the activator sequence is obtained from the B42 transcriptional activation domain (Ma and Ptashne, 1987, Cell 51:113-119). The promoters of the reporter genes contain LexA binding sequences and hence will be activated by the reconstitution of the transcriptional activator. Another feature of this system is that the gene encoding the DNA-binding domain fusion protein is under the influence of an inducible GAL promoter so that confirmatory tests can be performed under inducing and non-inducing conditions.

In another version of this system developed by Elledge and colleagues, the reporter genes HIS3 and lacZ (Durfee et al., 1993, Genes Dev. 7:555-569) are used. The transcriptional activator that is reconstituted in this case is GAL4 and protein-protein interactions allow cells to grow in media lacking histidine and containing 3-aminotriazole (3-AT) and to express β-galactosidase. 3-AT inhibits the growth of his3 auxotrophs in media lacking histidine (Kishore and Shah, 1988, Ann. Rev. Biochem. 57:627-663).

In another version of the two-hybrid assay, a URA3 reporter gene under the control of Estrogen Response Elements (ERE) has been used to monitor protein-protein interactions. Here, the DNA-binding domain is derived from the human estrogen receptor. The authors of the ERE assay propose that inhibition of the protein-protein interactions can be identified by negative selection on 5-FOA medium (Le Douarin et al., 1995, Nucleic Acids Res. 23:876-878), but do not provide any details.

A mammalian version of the two-hybrid approach called the “Contingent Replication Assay” that is applicable in mammalian cells has also been reported (Nallur et al., 1993, Nucleic Acids Res. 21:3867-3873; Vasavada et al., 1991, Proc. Natl. Acad. Sci. USA 88:10686-10690). In this case, the reconstitution of the transcription factor in mammalian cells due to the interaction of the two fusion proteins leads to the activation of the SV40 T antigen. This antigen allows the replication of the activation domain fusion plasmids. Another mammalian version of two-hybrid approach is the “Karyoplasmic Interaction Selection Strategy” that also uses the reconstitution of a transcriptional activator (Fearon et al., 1992, Proc. Natl. Acad. Sci. USA 89:7958-7962). Reporter genes used in this case have included the gene encoding the bacterial chloramphenicol acetyl transferase, the gene for cell-surface antigen CD4, and the gene encoding resistance to Hygromycin B. In both of the mammalian systems, the transcription factor that is reconstituted is a hybrid transcriptional activator in which the DNA-binding domain is from GAL4 and the activation domain is from VP16.

The next variations of the two-hybrid assays are adapted to examine the specificity of the binding by examining the reconstitution of a transcriptional activator having one domain fused to the candidate engineered binding domain and the other domain fused to a wide variety of other possible target proteins. In a variation of the “Interaction Trap” system, a “mating-grid” strategy has been used to characterize interactions between proteins that are thought to be involved in the Drosophila cell cycle (Finley and Brent, 1994, Proc. Natl. Acad. Sci. USA 91:12980-12984). This strategy is based on a technique first established by Rothstein and colleagues (Bendixen et al., 1994, Nucleic Acids Res. 22:1778-1779) who used a yeast-mating assay to detect protein-protein interactions. Here, the DNA-binding and activation domain fusion proteins were expressed in two different haploid yeast mating strains, a and α, and the two were brought together by mating. Thus, interactions between a candidate domain and other proteins can be studied in this method.

### Library-Pair Reconstitution Methods

The following reconstitution methods are primarily adapted to screen one library for interaction with a selected protein, or to screen two libraries for all pair-wise interactions.

Libraries for specificity screening can be constructed by the methods of Stanley Fields and coworkers who have recently performed an analysis of all possible protein-protein interactions that can take place in the E. coli bacteriophage T7 (Bartel et al., 1996, Nature Genet. 12:72-77). Randomly sheared fragments of T7 DNA were used to make libraries in both the DNA-binding domain and the activation domain plasmids and a genome-wide two-hybrid assay was performed by use of a mating strategy. The DNA-binding and the activation domain fusions were transformed into separate yeast strains of opposite mating type. The DNA-binding domain hybrids containing yeast transformants were then divided into groups of 10. The groups were screened against a library of activation domain hybrids numbering around 10⁵ transformants. By this method, 25 interactions were characterized among the proteins of T7. Such studies provide a method to screen more than one DNA-binding domain hybrid against more than one activation domain hybrid.

The following methods characterize a population or library of proteins by comparing all detectable protein-protein interactions that occur in a population or library with those interactions that occur in another population or library. These methods are described in U.S. Pat. No. 6,083,693, by Nandabalan and Rothberg, and U.S. Pat. No. 6,057,101, by Nandabalen, Rothberg, Yang, Knight, and Kalbfleisch. In the following, one of the populations may be fusions of the candidates to be assayed and the other population may be fusions of the new target peptides or proteins as well as including fusions of other cellular proteins. These methods can be applied to the parallel screening of a plurality of candidate binding domains for binding to a plurality of targets. Thereby both affinity and specificity may be determined in a single assay.

In these methods, protein-protein interactions are detected by measuring transcriptional regulation (preferably activation) that occurs upon interaction of proteins between the two populations being tested (referred to hereinafter merely for purposes of convenience as the M population and the N population). Proteins of each population (M, N) are provided as fusion (chimeric) proteins (preferably by recombinant expression of a chimeric coding sequence carried in a vector) containing each protein contiguous to a preselected sequence. For one population, the preselected sequence is a DNA binding domain. The DNA binding domain can be any available, as long as it specifically recognizes a DNA sequence within a promoter. For example, the DNA binding domain is of a transcriptional activator or inhibitor. For the other population, the preselected sequence is an activator or inhibitor domain of a transcriptional activator or inhibitor, respectively.

In one example, each protein in one population (e.g., M) is provided as a fusion to a DNA binding domain of a transcriptional regulator (e.g., activator). Each protein in the other population (N) is provided as a fusion to an activator domain of a transcriptional activator. The regulatory domain alone (not as a fusion to a protein sequence) and the DNA-binding domain alone (not as a fusion to a protein sequence) generally do not detectably interact (so as to avoid false positives in the assay). When binding occurs of a fusion protein in M to a fusion protein in N, reconstitution of a transcriptional activator occurs such that transcription is increased of a gene (“Reporter Gene”) responsive to (whose transcription is under the control of) the transcriptional activator. Thus, the Reporter Gene comprises a nucleotide sequence operably linked to a promoter regulated by a DNA binding site for the DNA binding domain of the transcriptional activator. The activation of transcription of the Reporter Gene occurs intracellularly, e.g., in prokaryotic or eukaryotic cells, preferably in cell culture.

The Reporter Gene comprises a nucleotide sequence operably linked to a promoter that is operably linked to one or more nucleic acid binding sites that are specifically bound by the DNA binding domain of the fusion protein that is employed in the assay of the invention, such that binding of a reconstituted transcriptional activator or inhibitor to the one or more DNA binding sites increases or inhibits, respectively, transcription of the nucleotide sequence under the control of the promoter. The promoter that is operably linked to the nucleotide sequence can be a native or non-native promoter of the nucleotide sequence, and the DNA binding site(s) that are recognized by the DNA binding domain portion of the fusion protein can be native to the promoter (if the promoter normally contains such binding site(s)) or non-native. Thus, for example, one or more tandem copies (e.g., 4 or 5 copies) of the appropriate DNA binding site can be introduced upstream of the TATA box in the desired promoter (e.g., in the area of position −100 to −400). In one example, four or five tandem copies of the 17 bp UAS (GAL4 DNA binding site) are introduced upstream of the TATA box in the desired promoter, that is in turn upstream of the desired coding sequence that encodes a selectable or detectable marker. In an example, the GAL1-10 promoter is operably fused to the desired nucleotide sequence; the GAL1-10 promoter already contains five binding sites for GAL4. Thus, in a particular example, the transcriptional activation binding site of the desired gene(s) are deleted and replaced with GAL4 binding sites (Bartel et al., 1993, BioTechniques 14(6):920-924; Chasman et al., 1989, Mol. Cell. Biol. 9:4746-4749). Referring to use of a particular gene as a Reporter Gene herein thus means that, if the native promoter is not driven by binding site(s) recognized by the DNA binding domain used in the interaction assay of the invention, such DNA binding site(s) have been introduced into the gene.

The Reporter Gene preferably comprises a nucleotide sequence, whose transcription is regulated by the transcriptional activator, that is a coding sequence that encodes a detectable marker or selectable marker, facilitating detection of transcriptional activation, thereby detecting a protein-protein interaction. The assay is typically carried out in the absence of background levels of the transcriptional activator (e.g. in a cell that is mutant or otherwise lacking in the transcriptional activator). Preferably, more than one different Reporter Gene is used to detect transcriptional activation, e.g., one encoding a detectable marker, and one or more encoding different selectable markers. The detectable marker can be any molecule that can give rise to a detectable signal, e.g., an enzyme or fluorescent protein. The selectable marker can be any molecule that can be selected for its expression, e.g., which gives cells a selective advantage over cells not having the selectable marker under appropriate (selective) conditions. The Reporter Gene used can be a gene containing a coding sequence whose native promoter contains a binding site for the DNA binding protein. Alternatively, the gene can be a chimeric gene containing a sequence that is transcribed under the control of a promoter that is not the native promoter for the transcribed sequence.

To make the fusion constructs (encoding the fusion proteins such that the fusion proteins are expressed in the desired host cell) from each library of population, the activation domain and DNA binding domain of a wide variety of transcriptional activator proteins can be used, as long as these transcriptional activators have separable binding and transcriptional activation domains. For example, the GAL4 protein of S. cerevisiae, the GCN4 protein of S. cerevisiae (Hope and Struhl, 1986, Cell 46:885-894); the ARD1 protein of S. cerevisiae (Thukral et al., 1989, Mol. Cell. Biol. 9:2360-2369), and the human estrogen receptor (Kumar et al., 1987, Cell 51:941-951) have separable DNA binding and activation domains. The DNA binding domain and activation domain that are employed in the fusion proteins need not be from the same transcriptional activator. In one example, a GAL4 or LEXA DNA binding domain is employed. In another example, a GAL4 or herpes simplex virus VP16 (Triezenberg et al., 1988, Genes Dev. 2:730-742) activation domain is employed. Amino acids 1-147 of GAL4 (Ma et al., 1987, Cell 48:847-853; Ptashne et al., 1990, Nature 346:329-331) is the DNA binding domain, and amino acids 411-455 of VP16 (Triezenberg et al., 1988, Genes Dev. 2:730-742; Cress et al., 1991, Science 251:87-90) is the activation domain.

The host cell in which the interaction assay occurs can be any cell, prokaryotic or eukaryotic, in which transcription of the Reporter Gene can occur and be detected, including but not limited to mammalian (e.g., monkey, chicken, mouse, rat, human, bovine), bacteria, and insect cells, and is preferably a yeast cell. Expression constructs encoding and capable of expressing the binding domain fusion proteins, the transcriptional activation domain fusion proteins, and the Reporter Gene product(s) are provided within the host cell, by mating of cells containing the expression constructs, or by cell fusion, transformation, electroporation, microinjection, etc. For example, GAL4 and VP16 are functional in animal cells and thus the desired binding or activation domain thereof can be used in, e.g., yeast or mammalian cells. Various DNA binding domains, activation domains, promoters, and/or DNA binding sites can be used in these methods, as long as the DNA binding sites are recognized by the DNA binding domains, and the promoter is operative in the cells chosen in which to carry out the assay of the invention. The host cell used should not express an endogenous transcription factor that binds to the same DNA site as that recognized by the DNA binding domain fusion population. Also, preferably, the host cell is mutant or otherwise lacking an endogenous, functional form of the Reporter Gene(s) used in the assay.

In one example, transcription of the Reporter Gene is detected by a linked replication assay. For example, as described by Vasavada et al. (1991, Proc. Natl. Acad. Sci. USA 88:10686-10690), for use in animal cells, a Reporter Gene under the control of the E1B promoter, which promoter in turn is controlled by GAL4 DNA binding sites, encodes the SV40 T antigen. In the presence of reconstituted GAL4 DNA binding domain-activation domain (composed of two interacting fusion proteins), SV40 T antigen is produced from the Reporter Gene. If a plasmid is present that contains the SV40 origin of replication, this plasmid will replicate only upon the production of SV40 T antigen. Thus, replication of such a plasmid is used as an indicator of protein-protein interaction. Constructing one or both of the plasmids encoding the fusion proteins of the assay to contain an SV40 origin of replication means that replication of these plasmids will be an indication of Reporter Gene activity. Sensitivity to DpnI can be used to destroy unreplicated plasmids according to the methods described in Vasavada et al. (1991, Proc. Natl. Acad. Sci. USA 88:10686-10690). In an alternative example, alternatively to an SV40 origin of replication, a polyoma virus replicon is employed (id.)

Preferably, the protein-protein interactions are assayed according to the method of the invention in yeast cells, e.g., Saccharomyces cerevisiae or Schizo-saccharomyces pombe. Various vectors for producing the two fusion protein populations and host strains for conducting the assay are known and can be used (see, e.g., Fields et al., U.S. Pat. No. 5,468,614 dated Nov. 21, 1995; Bartel et al., 1993, “Using the two-hybrid system to detect protein-protein interactions,” in Cellular Interactions in Development, Hartley, D. A. (ed.), Practical Approach Series xviii, IRL Press at Oxford University Press, New York, N.Y., pp. 153-179; Fields and Sternglanz, 1994, TIG 10:286-292). Many other strains commonly known and available in the art can be used. If not already lacking in endogenous Reporter Gene activity, cells mutant in the Reporter Gene are selected by known methods, or the cells are made mutant in the target Reporter Gene by known gene-disruption methods prior to introducing the Reporter Gene (Rothstein, 1983, Meth. Enzymol. 101:202-211).

### Direct Observation of Binding

As used herein, a method directly observes binding if it does not depend on the intervention of any reporter gene or physiologic change. Certain of these methods physically observe binding between a pair of proteins (or a protein and a peptide, or a protein and a ligand), such as affinity chromatography or biosensor analysis; other of these methods use constructs linking a protein to its genetic description, such as phage display or protein-RNA hybrids. Included among assay methods depending on directly observing binding are primarily physical methods such as affinity chromatography and the like, isothermal calorimetry and the like, biosensor analysis, and so forth. Also included are more biological methods such as phage display, RNA-protein fusions, and so forth.

### Affinity Chromatography

Affinity chromatography (also known as bio-selective adsorption) is both a protein purification and assay technique, and may therefore be used not only for protein binding assays, but also for protein purification. Once a binding domain has been engineered to the N- or C-terminus of a (naturally-occurring) protein, it can be used in the methods of affinity chromatography to assay for and to purify that protein.

In general, affinity chromatography can be effectively used to assay for the affinity or specificity of a candidate domain for a new target. For assaying affinity, either the candidate domain or the new target is covalently attached to a column; then the other partner is added to this column; next the column is washed with buffers of varying ionic strengths to partially remove any unbound partner; next the remaining bound candidate domain is eluted by (for example) adding a high concentration of a buffer suitable to release the binding-domain-target complex (or a high concentration of the bound partner, or an analogue of the bound partner, or a binding partner with higher affinity); and finally, the presence of the candidate domain is analyzed by a method for detecting specific proteins, such as Western blot. In this manner, binding of the candidate domain and the new target can be assayed as a function of the strength of the buffer, yielding indications of the binding affinity. For assaying specificity, preferably the candidate domain is covalently attached to a column; then a mixture of proteins including the new target is added to this column, which is then washed with buffer to remove unbound proteins; next the bound protein is eluted; and finally, the presence of the new target is analyzed by a method such as Western blot. In this manner, the specificity of the binding of the candidate domain can by assayed.

The following key elements for the method include the matrix, the solvents, spacer arms (if any), matrix coupling methods, and the methods of pouring the column. These elements are described in turn. Samples of the candidate engineered binding domain and the new target, if necessary for analysis, may synthesized, for example, by the methods described in the next section.

A sound matrix is an essential part of affinity chromatography. A matrix, in its use here, is a substance, usually in bead form, to which a specific ligand is covalently bound. In order for the matrix to be effective it must have certain characteristics: 1) it must be insoluble in solvents and buffers employed in the process; 2) it must be chemically and mechanically stable; 3) it must be easily coupled to a ligand or spacer arm onto which the ligand can be attached; 4) it must exhibit good flow properties and have a relatively large surface area for attachment. It is common for matrices to be made out of agarose, glass, cellulose, or a dual composition polyacrylimide based compound. See generally, e.g., Scouten, 1981, Affinity Chromatography, New York: John Wiley & Sons.

The primary buffer in affinity chromatography is the one in which the matrix resides. This buffer should not degrade the matrix in any way. The buffer should also have a negligible effect on the sample. The ideal buffer minimizes nonspecific interactions while maximizing the specific interaction between the sample and the ligand. The other major solvent to consider in affinity chromatography is the elution buffer. The purpose of the elution buffer is to wash away unbound proteins initially and at higher concentration release the desired protein from the ligand. Salt solutions of various concentrations, various detergents, as well as buffers containing specific analogs for the sample can be used. It is important that the elution buffer work quickly and to not change the function or activity of the desired protein.

Spacer arms, though not always necessary, can improve binding probability. Spacer arms distance the ligand from the matrix reducing steric hindrance which can occur, especially when the new target is bound directly to the bead. Spacer arms should neither chemically or structurally affect the sample or the ligand, See, Scouten, supra. When coupling the spacer arm or ligand to the matrix, multipoint or single point attachment may be used. Single point attachment offers high ligand flexibility and easier ligand access to the sample's active site. Though single point coupling does provide better site recognition, it is not nearly as strong as a multipoint attachment. Multipoint coupling is stronger than single point attachment and will thus show less degradation. Unfortunately it can impede binding between the ligand and the sample. After the ligand has been bound to the matrix it is important as a final step to block all unreacted groups of the matrix. This provides a higher degree of certainty that all binding will be between the sample and the ligand. See, e.g., Deutscher, 1990, Methods in Enzymology: Guide to Protein Purification, San Diego: Academic Press Inc., 1990.

Coupling or immobilization of either a candidate domain or a target to the column matrix or beads (with or without spacer arms) can be accomplished by standard methods. For example, either the matrix or the candidate domain (or target) can be activated for mutual covalent coupling. Alternatively, the candidate domain (or target) can be labeled with biotin and the streptavidin coupled to the matrix for their (essentially irreversible) binding. See generally, e.g., Harlow et al., 1988, Antibodies A Laboratory Manual, Cold Spring Harbor, N.Y.: Cold Spring Harbor Laboratory (especially chapter 13). Similar coupling methods can be used as are employed for biosensor analysis. See infra.

Once the ligand (either the new target or a candidate binding domain) has been immobilized, the column packed, and the sample prepared the sample can finally be poured. Since ligand binding is based on hydrogen bonding, site/ligand motifs and other non-covalent interaction, a certain degree of care must be used to make binding as opportune as possible. Variations in flow rate of the sample, and of the wash and elution buffers can exhibit monumental effects on the success of affinity chromatography. If the sample is poured too quickly proper binding may not take place. If, when pouring the wash, the flow rate is too high the bound protein may release as well. And finally, though the elution may be at a higher flow rate, if it exceeds the rate used to pack the original matrix the entire complex may come apart. See, Deutscher, supra.

Just as important as flow rate is the concentration and acidity of the buffers used. As mentioned above, buffers should in no way chemically or structurally interfere with ligand binding. In the elution phase it is necessary for buffer to separate the ligand from the sample, but prior to that buffers should remain innocuous because of the sensitive ligand/sample interaction.

Biosensor Analysis

Optical biosensors are emerging as important tools for characterizing the interactions of biological macromolecules, and have immediate application in this invention to assaying for the specificity and affinity of interaction of a candidate engineered binding domain and its new target peptide or protein. Biosensors can provide qualitative information on the macromolecular interaction processes under a variety of conditions. Quantitative information, in the form of affinity constants for complex formation, can be obtained in a manner similar to conventional solid-phase assays. In this subsection, the term “macromolecule” is to be understood in this subsection to mean a candidate engineered binding domain or a new target for such a domain, either a peptide of a protein having that peptide as a terminal sequence; and the terms “analyte” and “target” can mean either the new target, or the candidate domain, or a mixture of proteins optionally including the candidate domain or the new target. For example, if a candidate domain is used as a “target”, then the new target is the “analyte”, and vice versa. The biosensor output of primary interest herein is the affinity and specificity of binding of a candidate domain and its new target; other parameters such as the kinetics of binding are of less interest.

The underlying principle of biosensors, developed earlier with such techniques as analytical affinity chromatography, is twofold: (i) the use of a ligand immobilized on a solid phase as an affinity surface to bind one or more soluble analytes; and (ii) the measurement of mass migration on the affinity surface to quantitate the analyte's ligand binding properties. Biosensors operate by optically detecting mass buildup on a sensor surface when the analyte binds to immobilized ligand. Depending on the objective of each study, there are fundamentally two levels of information that can be obtained from biosensor experiments. The first is whether a given set of molecules bind to each other and the relative affinity and specificity of the interaction. The second level is the quantitative analysis of specific interaction kinetics. For the majority of biosensor experiments, in particular to obtain kinetic information, the homogeneity of the samples is the key to success, as in many other biochemical assays.

The basic method of using biosensors involves the steps of ligand immobilization, analyte binding, analyte dissociation and regeneration to prepare the sensor surface for the next binding cycle. The solvent environment of the ligand and analyte, including the running buffer, plays a major role in modulating the specificity of the interactions and the resultant measured rate constants. The running buffer must enable association and dissociation to occur between analyte and ligand.

Changes in running buffer, such as additives in the analyte sample, may introduce changes in reflective index that are observed as bulk phase signal. The latter needs to be differentiated from binding signal. As a general rule, the generic nature of the optical detection system necessitates vigilance to minimize the contribution of buffer conditions to nonspecific refraction. For examples, samples stored in phosphate buffers with divalent cations such as Ca²⁺ may produce precipitates with detectable refractive index changes. Hepes-buffered saline (HBS) and phosphate-buffered saline (PBS) (pH 7.4) are typically recommended by biosensor manufacturers, but there are no fundamental restrictions to the use of other compatible buffers. In general, running buffer should be filtered (0.22-μm filter) and thoroughly degassed to avoid increased noise, spikes, and baseline fluctuations. The temperature of running buffer and that of analytes should remain constant throughout the whole binding interaction process. Low levels of nonionic detergent (i.e., 0.005% P-20 or Tween 20) are recommended to reduce adsorption of bulk-phase molecules to the integrated microfluidics cartridge (“IFC”). The use of components with high refractive index, such as glycerol and dimethylsulfoxide (DMSO), should be minimized. The buffer composition can be adjusted to reduce nonspecific interaction of analyte with the sensor surface, for example, by increasing NaCl concentration (by 50-100%) to reduce ionic interactions with a carboxydextran surface or by increasing surfactant content (by 10-fold) to reduce hydrophobic interactions with an alkyl surface.

The available immobilization methods are categorized into two general groups: direct covalent coupling and affinity capture via immobilized anchor. Covalent coupling effects a more stable baseline by ensuring that immobilized ligands do not dissociate from the surface or exchange with other proteins in solution. The amine coupling method is used most commonly as an initial approach, unless there is a reason to suspect that blocking critical amino groups during the procedure will inactivate the ligand. Covalent coupling generally is achieved in four steps: a preconcentration at the matrix to drive the ligand into the hydrogel; activation (3-8 min); covalent bond formation at the same pH and at low ionic strength (<50 mM NaCl or equivalent for other buffers); blocking of activated carboxyl groups that have not been hydrolyzed (3-8 min); and regeneration of the immobilized ligand sensor surface in preparation for adding analyte. Because the net charge of the most frequently used carboxydextran matrix (Biacore) is negative, ligands often are preconcentrated at a pH below their isoelectric point. Superactivation of surface carboxyls, by increasing the recommended concentrations of NHS/EDC 5- to 10-fold, reduces the net negative charge of the hydrogel and hence can regulate the preconcentration effect. The slope of the increasing signal due to accumulation of mass in the preconcentration phase should be linear; this slope enables an estimate to be made of the contact time required to obtain a predetermined number of resonance units of immobilized ligand. Whichever method is employed to coat the sensor, the coated surface should be washed thoroughly to eliminate non-specifically immobilized ligand and to ensure that the amount of immobilized ligand will remain relatively constant throughout the experiment. The constant response or R value of the coated and washed sensor surface becomes the baseline for subsequent binding experiments.

The concentration of immobilized ligand may be estimated from the dimensions of the flow cell, the thickness of the hydrogel, and the baseline signal change. The SPR detection area is 0.224 mm²(1.4×0.16 mm), and the carboxydextran hydrogel height is 100 nm (20 nm in 150 mM NaCl). The R-to-mass correlation is 1 response unit for each 1 pg/mm² bound to the surface. Thus, 1 RU is equivalent to a surface concentration of 10 mg/liter. A 30-kDa protein immobilized at a concentration of 333 nmol/liter or 333 nM would be expected to generate a signal of approximately 100 RU. The baseline signal after the immobilization, also referred to as the immobilization level, represents the binding capacity of the ligand. In general, to answer the “yes or no” question of whether the analyte binds specifically to a given ligand, high immobilization may be advantageous to increase the signal. However, for the purpose of kinetic analysis, low immobilization levels offer more reliable results for the kinetic analysis of the data.

The association and dissociation of an analyte with the immobilized ligand are monitored by changes in R that occur in real time once the analyte has been introduced into the flow upstream of the sensor surface and subsequently when the flow cell is washed with running buffer alone. In the association phase, the analyte sample is injected over the surface at a constant flow rate for a defined contact time. Any mass increase on the surface resulting from ligand-analyte interaction is detected as an increase in response. To obtain the maximum information about the interaction, a wide range of analyte concentrations should be used in each experiment. Testing different flow rates and contact times often provides additional valuable kinetic information.

In the dissociation phase, a wash with the running buffer is applied after the analyte sample injection has been completed. The dissociation of analyte from the ligand-analyte complex is detected as a decrease in signal due to loss of mass from the surface. The dissociation phase is intended to remove all the non-covalently bound material, allowing the baseline to return to the initial value before the analyte injection. Because the number of free sites increases with the time of dissociation, there may be a residual signal due to rebinding of the analyte. To test for this artifact, it is recommended that (a) excess soluble ligand be included in the running buffer during the dissociation phase, and (b) the surface density of covalently attached ligand be reduced. The return of the signal to baseline is a useful indicator that irreversible interaction with the hydrogel or the capture molecules has not occurred.

After an analyte association-dissociation cycle, the sensor surface is washed to regenerate the starting ligand-immobilized sensor surface to prepare for the next interaction cycle. Running buffer wash may not remove the analyte completely and return the signal to baseline. On the other hand, removing residual bound analyte and nonspecifically adsorbed molecules must be accomplished without affecting the activity of the immobilized ligand. Brief regeneration with a chaotropic agent, repeated if necessary, offers the best approach. A rule of thumb is to use the mildest possible regeneration that will return signal to baseline. Increased harshness of the regeneration procedure generally reduces the lifetime of the immobilized ligand sensor surface.

To ensure the reliability of biosensor data, binding experiments should be repeated with more than one condition. A dose response at concentrations of analyte over a range of several orders of magnitude will help to confirm the maximum binding capacity (or saturability) of the surface. Negative and positive controls are critical additional measures of the reliability of binding data. A reference surface is always needed to account for and to minimize artifacts. Reruns of analyte periodically during a binding series ensure the integrity of the sensor surface.

### Physical Methods

The present invention also encompasses the use of physical methods to measure and monitor binding of redesigned polypeptides to their new target peptides or peptide sequences.

Physical methods for measuring the dissociation (or affinity) constant include iso-thermal calorimetry, surface plasmon resonance, analytical ultra-centrifagation (where appropriate), and so forth. Iso-thermal calorimetry is the preferred method. Iso-thermal calorimetry measures the heat released when a binding polypeptide binds to its target under constant-temperature, constant-pressure conditions. From these measurements, the thermodynamic parameters of this binding can be routinely determined by the methods of physical chemistry. For example, it is routinely possible to obtain the binding affinity (or dissociation constant), the stoichiometry of binding (n), the heat (or enthalpy) of binding (H), the heat capacity of binding, the entropy of binding, and so forth. Current micro-iso-thermal machines are readily available commercially. Such machines are fully automatic, directly returning the above thermodynamic results. See, e.g., MicoCal, LLC, Northampton. Mass.; www.microcalorimetry.com.

Other physical methods may be of use in the present invention. For example, a spatial structure of the redesigned polypeptides in complex with its new target may serve as a starting point for its further redesign. Spatial structures can be obtained by X-ray crystallography or NMR (for smaller molecules). Further, instead of a complete structure determination, NMR may be used to determine the structure of only in the binding domain/pocket and surroundings, both in complex with the new target and free. Alternatively, NMR may determine changes in the position of binding domain residues upon binding. This additional spatial structure may be used in further iterations of the rational methods of this invention, for example, providing improved backbone structures for the binding polypeptide.

### Phage Display

Phage display techniques simply and directly link a phage genotype with the protein phenotype displayed on the phage surface. In phage display, bacteriophage particles are made by standard techniques with either a peptide or protein of interest fused to a capsid or coat protein and with an enclosed fusion DNA that encodes the coat protein fusion. Often, phage display uses the M13 filamentous, single-stranded DNA phage. Its replication with a fusion coat protein gene is induced with the aid of a helper phage with a wild-type coat protein gene but with deficient phage packing signals.

In practice, phage are prepared by standard methods expressing a library of “prey” proteins on their surface. Any ‘bait’ protein can then be immobilized to capture phage particles displaying interacting “prey” on their surfaces. The identity of the interacting “prey” can then be determined by, for example, culturing of bound phage followed by sequencing of their DNA, or by PCR amplification of bound phage DNA along with sequencing, or by other methods. Phage display is similar to the yeast two-hybrid system, in that it is simple and can be performed with throughput, but can have advantages in certain cases. Depending on the particular class of proteins being studied (such as cytoplasmic versus cell surface proteins), this method may be superior or inferior to the two-hybrid system because the interactions take place in vitro as opposed to the in vivo interactions in the nucleus of the yeast cell.

This technique has been used to screen for peptide epitopes, peptide ligands, enzyme substrates or single-chain antibody fragments. Although combinatorial peptide libraries have generally been used in most phage display-based studies, large-scale protein interaction studies can now be done if the products of cDNA libraries are displayed on phage particles. Furthermore, this method is applicable in principle to transcription factors, which are not amenable to the yeast two-hybrid system. Methods have recently been optimized to display cDNA libraries on phages to isolate signaling molecules in the EGF-receptor signaling pathway (See Zozulya, S. et al. Nature Biotechnol. 17, 1193-1198 (1999)) as well as to identify antigens that react with certain antibodies (See Hufton, S. E. et al. J. Immunol. Methods 231, 39-51 (1999)).

As applied in the present invention, phage display may be used to assess both affinity and specificity of candidate domain binding to new targets. In one embodiment, a combinatorial peptide library (including the target peptide) is displayed on the phage surfaces, and one or more (immobilized) candidate domains are contacted with the phage library to assay domain binding and binding specificity to library members. Alternatively, the phage may display a cDNA library products (including the target protein) in order to directly access binding to a target protein. In another embodiment, a library of engineered candidate domains may be screened against one or more (immobilized) new targets, or against a library of targets to assess binding (and in various binding conditions to assess binding affinity).

### RNA-Protein Fusions

RNA-protein fusions are similar to phage display techniques in that this fusion links a protein “phenotype” directly, even covalently, to its encoding “genotype”. A simpler and robust system (Roberts, R. W. & Szostak, J. W. (1997) Proc. Natl. Acad. Sci. USA 94, 12297-12302) is available for use with the current invention in which an mRNA would become directly attached to the peptide or protein it encodes by a stable covalent linkage. Covalent fusions between an mRNA and the peptide or protein that it encodes can be generated by in vitro translation of synthetic mRNAs that carry puromycin, a peptidyl acceptor antibiotic, at their 3′ end. The stable linkage between the informational (nucleic acid) and functional (peptide) domains of the resulting joint molecules allows a specific mRNA to be enriched from a complex mixture of mRNAs based on the properties of its encoded peptide. In this way, the sequence information present in the peptide would be encoded in the covalently attached mRNA. This joint molecule then could act as a molecular Rosetta stone, allowing information in the protein portion to be read and recovered via the attached mRNA.

This approach using covalent RNA-peptide fusions is expected to provide an additional route to the in vitro selection and directed evolution of proteins. Because proteins carry out a wider range of structural and catalytic roles in biology and are much more extensively used in diagnostic, therapeutic, and industrial applications, great interest has been generated in the development of methods for the in vitro selection and directed evolution of proteins. The main barrier to the development of effective methods for protein evolution has been the difficulty of recovering the information encoding a protein sequence after the protein has been translated. In particular, in vitro selection experiments using RNA and DNA have shown that nucleic acid molecules with specific molecular recognition and catalytic properties can be isolated from complex pools of random sequences by repeated rounds of selection and amplification (Breaker, R. R. (1997) Chem. Rev. (Washington, D.C.) 97, 371-390). Directed in vitro evolution, in which mutagenic amplification is combined with continued selective pressure, has been widely used to select for nucleic acids with altered or improved binding or catalytic properties (Bartel, D. P. & Szostak, J. W. (1993) Science 261, 1411-1418).

However, RNA-protein fusions may be applied in the present invention, similarly to phage displays, in order to assess both affinity and specificity of candidate domain binding to new targets. In one embodiment, a library of RNA-peptide fusions, where the peptides form a substantially combinatorial library including the target peptide, is contacted to one or more immobilized candidate domains. Any bound fusions are then released, and the RNA moieties are analyzed (for example, by PCR and/or sequencing) in order to determine target binding and candidate binding specificity. Alternatively, the fusions may form a library formed from expressed mRNA (including the target protein) in order to directly access binding to a target protein. In another embodiment, the fusions are formed from mRNAs encoding a library of two or more engineered candidate domains, and are contracted to one or more (immobilized) new targets, or against a library of targets, to assess binding (and in various binding conditions to assess binding affinity).

In a further application, mRNA encoding candidate domains, or domains determined to be acceptable, may include at the 5′ end a marker segment for identification by hybridization with DNA oligonucleotides. For example, a marker segment containing an oligo-ribonucleotide code for the attached binding domain may be attached 5′ to the 5′-untranslated region in order not to disturb domain translation. Alternately, it may be attached in-frame after the start codon in order to be translated into a leader peptide on the binding domain. Then selection, attachment, or identification of particular RNA-binding-domain fusions could use well known and reliable oligonucleotide technology.

### Domain Synthesis

The discussion below focuses principally on production of the precursor binding domain of interest (also referred to in this subsection simply as the “binding domain”) or candidate engineered binding domain (also referred to in this subsection as the “binding domain variant”) (collectively referred to as the “domains of interest”) by culturing cells transformed with a vector containing the nucleic acid encoding the binding protein of interest or binding protein variant and recovering the binding protein of interest or variant from the cell culture (generically referred to herein as “biotechnology” or “genetic engineering” methods). This preferred synthesis method includes the two basic steps of preparing DNA encoding the proteins of interest and then causing the expression of the prepared DNA

### Preparation/Mutagenesis of Encoding DNA

Simply, both DNA encoding the precursor portion and DNA encoding the candidate domains may be obtained by in vitro oligonucleotide synthesis. In this case, the following steps of gene selection and mutagenesis are not necessary. If in vitro oligonucleotide synthesis is not possible, practical, or economical, then the following processes are preferred.

Generally, the primary sequence of any binding domain, and of the protein including the binding domain (“precursor” protein) if the binding domain is a fragment, is known in advance (at least from protein databases consulted earlier). DNA encoding the “precursor” protein (the “precursor” gene) may preferably be obtained from a cDNA library prepared from tissue possessing mRNA encoding the precursor protein. DNA encoding the (precursor) binding domain may than be pulled from such a library with probes designed to identify the “precursor” gene, or more preferably to identify that portion of the precursor gene encoding the precursor binding domain (the “precursor” portion). In some embodiments, the nucleic acid sequence that includes the precursor protein is selected to include preceding signal sequences. Suitable probes are (synthesized) oligonucleotides of about 20-80 bases in length that are complementary to the initial and terminal pieces of the precursor portion, which are then used to PCR amplify the precursor. These probes may also be used to purify the amplified portion by an affinity process.

Oligonucleotide sequences selected as probes should be of sufficient length and sufficiently unambiguous that false positives are minimized. The oligonucleotide may be labeled such that it can be detected upon hybridization to DNA in the library being screened. The preferred method of labeling is to use ³²P-labeled ATP with polynucleotide kinase, as is well known in the art, to radio-label the oligonucleotide. However, other methods may be used to label the oligonucleotide, including, but not limited to, biotinylation or enzyme labeling.

Genomic libraries are less preferable for obtaining encoding DNA because it may be necessary to deal with introns in the precursor gene. Appropriate probes for screening genomic DNA libraries include, but are not limited to, oligonucleotides, cDNAs, or fragments thereof, that encode the same or a similar gene, and/or homologous genomic DNAs or fragments thereof.

Library screening is well known in the art. See, generally for these and other techniques described herein, Sambrook et al., 1989, Molecular Cloning: A Laboratory Manual, New York: Cold Spring Harbor Laboratory Press.

The variants of the binding protein, that is the various candidate binding domains of interest are suitably prepared by introducing the nucleotide changes determined by the methods of Sec 5.1 into the DNA encoding the binding protein of interest. Such variants are primarily substitutions of residues within the amino acid sequence of the binding protein of interest so that it contains an engineered domain for binding to its new target, but may also include insertions or deletions. Preferably precursor domains have no functionally significant post-translational modification. If such are present, amino acid changes that may unfavorably alter these post-translational processes, such as changing the number or position of glycosylation sites, should be avoided

In addition to the amino acid substitutions specifically related to engineering the new binding properties of the precursor domain, certain additional structural changes may be advantageous, including for example, the following. It may be desirable to inactivate one or more protease cleavage sites that are present in the molecule. These sites are identified by inspection of the encoded amino acid sequence, in the case of trypsin, e.g., for an arginyl or lysinyl residue. When protease cleavage sites are identified, they are rendered inactive to proteolytic cleavage by substituting the targeted residue with another residue, preferably a neutral polar residue such as glutamine or a hydrophilic residue such as serine, threonine, tyrosine or asparagine; by deleting the residue; or by inserting a prolyl residue immediately after the residue. Alternatively, about 1-3 residues are inserted adjacent to such sites. Any cysteine residues not involved in maintaining the proper conformation of the binding protein of interest also may be substituted, generally with serine or alanine, to improve the oxidative stability of the molecule and prevent aberrant cross-linking.

Nucleic acid molecules encoding amino acid sequence variants of the binding protein of interest are prepared by a variety of methods known in the art, which methods include, but are not limited to, preparation by oligonucleotide-mediated (or site-directed) mutagenesis, PCR mutagenesis, and cassette mutagenesis of an earlier prepared variant or a non-variant version of the binding protein on which the variant herein is based (“binding protein of interest”).

Oligonucleotide-mediated mutagenesis is a preferred method for preparing substitution, deletion, and insertion binding protein variants herein. This technique is well known in the art as described by Adelman et al., DNA, 2: 183 (1983). Briefly, the DNA is altered by hybridizing an oligonucleotide encoding the desired mutation to an oligonucleotide template, where the template is the single-stranded form of a plasmid or bacteriophage containing the unaltered or native DNA sequence of the binding protein to be varied. After hybridization, a DNA polymerase is used to synthesize an entire second complementary strand of the template that will thus incorporate the oligonucleotide primer, and will code for the selected alteration in the DNA. Generally, oligonucleotides of at least nucleosides in length are used. An optimal oligonucleotide will have 12 to 15 nucleosides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci. USA, 75: 5765 (1978).

The DNA template can be generated by those vectors that are either derived from bacteriophage M13 vectors (the commercially available M13mp18 and M13mp19 vectors are suitable), or those vectors that contain a single-stranded phage origin of replication as described by Viera et al. Meth. Enzymol., 153: 3 (1987). Thus, the DNA that is to be mutated may be inserted into one of these vectors to generate single-stranded template. Production of the single-stranded template is described in Sambrook et al., supra. Alternatively, single-stranded DNA template may be generated by denaturing double-stranded plasmid (or other) DNA using standard techniques.

For alteration of the original DNA sequence to generate the binding protein variants of this invention, the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of the binding protein, and the other strand (the original template) encodes the original, unaltered sequence of the binding protein. This heteroduplex molecule is then transformed into a suitable host cell, usually a prokaryote such as E. coli JM101. After the cells are grown, they are plated onto agarose plates and screened using the oligonucleotide primer radio-labeled with ³²P to identify the bacterial colonies that contain the mutated DNA. The mutated region is then removed and placed in an appropriate vector for protein production, generally an expression vector of the type typically employed for transformation of an appropriate host.

The method described immediately above may be modified such that a homoduplex molecule is created wherein both strands of the plasmid contain the mutation(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thio-deoxyribocytosine called dCTP-(.alpha.S) (which can be obtained from the Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(.alpha.S) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101, as described above.

DNA encoding binding protein mutants with more than one amino acid to be substituted may be generated in one of several ways. If the amino acids are located close together in the peptide chain, they may be mutated simultaneously using one oligonucleotide that codes for all of the desired amino acid substitutions. If, however, the amino acids are located some distance from each other (separated by more than about ten amino acids), it is more difficult to generate a single oligonucleotide that encodes all of the desired changes. Instead, one of two alternative methods may be employed.

In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions.

The alternative method involves two or more rounds of mutagenesis to produce the desired mutant. The first round is as described for the single mutants: wild-type DNA is used for the template, an oligonucleotide encoding the first desired amino acid substitution(s) is annealed to this template, and the heteroduplex DNA molecule is then generated. The second round of mutagenesis utilizes the mutated DNA produced in the first round of mutagenesis as the template. Thus, this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

PCR mutagenesis is also suitable for making amino acid variants of this invention. The PCR technique generally refers to the following procedure. See, e.g., Sciki et al, 1986, Nature 329 :163-166 . When small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into DNA, one of the primers is designed to overlap the position of the mutation and to contain the mutation; the sequence of the other primer must be identical to a stretch of sequence of the complementary strand. PCR amplification using a primer pair like the one just described results in a population of DNA fragments that differ at the position of the mutation specified by the primer, and possibly at other positions, as template copying is somewhat error-prone. If the ratio of template to product material is extremely low, the vast majority of product DNA fragments incorporate the desired mutation(s).

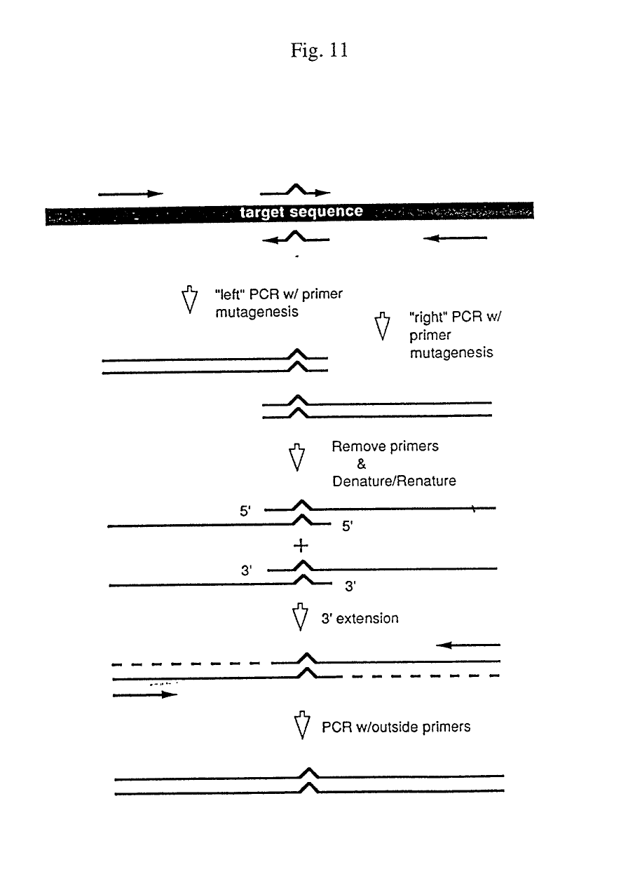

Shown in FIG. 11 are two PCR products that overlap in sequence; both contain the same mutation introduced as part of the PCR primers. These overlapping, primary products can be denatured and allowed to re-anneal together, producing two possible heteroduplex products. The heteroduplexes that have recessed 3′ ends can be extended by Taq DNA polymerase to produce a fragment that is the sum of the two overlapping products. A subsequent re-amplification of this fragment with only the right- and left-most primers (“outside” primers) results in the enrichment of the full-length, secondary product. In this way fragments containing the mutations far away from the fragment ends can be made by using PCR. DNA base methylation may also be used to control mutagenesis

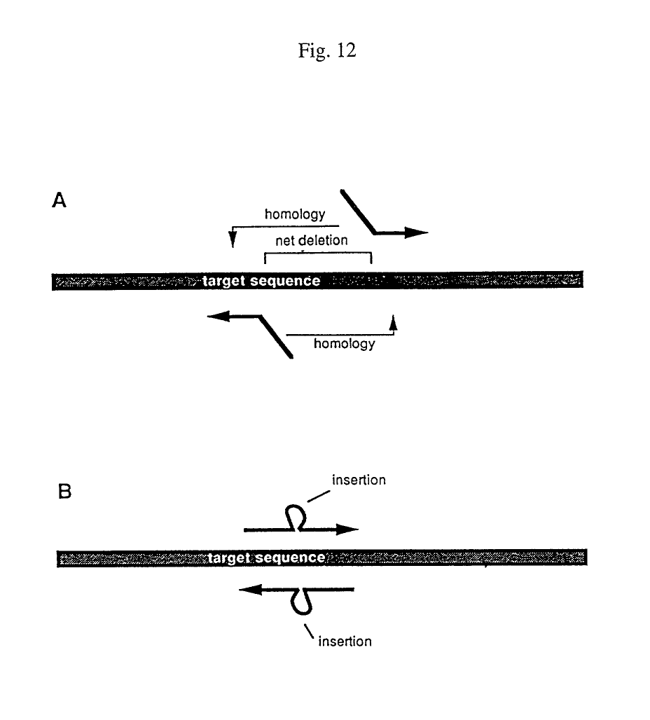

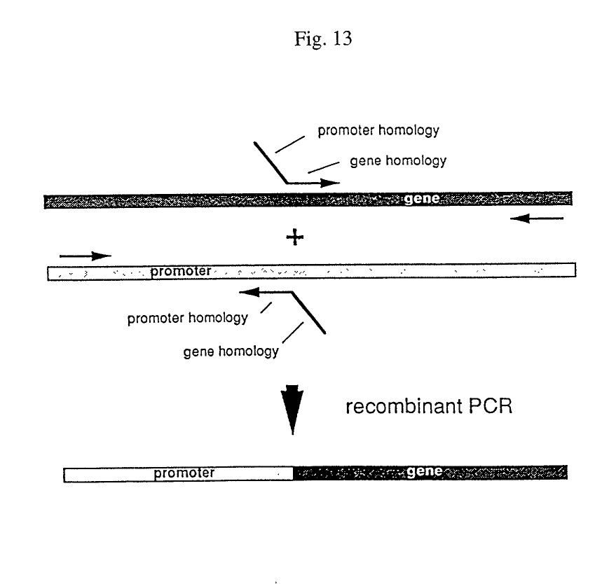

As with add-on mutagenesis, the mutation can be a base substitution, a small insertion, or a deletion. To produce a deletion, the primer sequence contains the deletion near the 5′ end such that anything 5′ of the deletion point is effectively add-on (see FIG. 12). The mutation needs only to be mirrored in each of the two primary PCR fragments. Note also that two previously unrelated DNA sequences can be joined this way at almost any position by using the 5′-add-on sequences as “adapters (FIG. 13), in which a hypothetical promoter sequence and gene are joined as precisely as desired by having the sequence of the junction region shared by the “inside” primers. The process has been called “recombinant PCR”.

To make a base substitution, inside primers are mismatched to the target sequence at the substituted base. The effect of this mismatch on PCR will be less the more 5′ the mismatch is. However, the more 3′ the mismatch is in one primer, the more 3′ it will be in the other inside primer (FIG. 11). Therefore the primers used to create substitutions in this way have the mismatch at their middle and have about nucleosides long.

To do add-on mutagenesis to create deletions or small insertions (see FIG. 12), or to combine sequences (FIG. 13), it is recommended to use inside primers with at least bases of target sequence homology that are 3′ to add-on sequences of to bases. A longer insertion may be possible with a longer add-on sequence. Very large insertions may be done by combining three PCR fragments, two flanking and one comprising the insertion sequence, using serial applications of recombinant PCR.

Another method for preparing variants, cassette mutagenesis, is based on the technique described by Wells et al., Gene, 34: 315 (1985). The starting material is the plasmid (or other vector) comprising the DNA to be mutated. The codon(s) in the DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are compatible with the ends of the linearized plasmid, such that it can be directly ligated to the plasmid. This plasmid now contains the mutated DNA sequence.

### Protein Synthesis

In a simple embodiment, candidate binding domains may also be synthesized by solid phase method (if they are not too long, approximately 100-150 residues or less). If directly synthesized, the preceding genetic and nucleic acid manipulations may be dispensed with. Protein synthesis is well known and even automated or commercially available, and is generally described by the following. Amino acids are added step-wise to a growing peptide chain that is linked to an insoluble matrix, such as polystyrene beads. A major advantage of this solid-phase method is that the desired product at each stage is bound to beads that can be rapidly filtered and washed and so the need to purify intermediates is obviated. All of the reactions are carried out in a single vessel, which eliminates losses due to repeated transfer of products. The carboxyl-terminal amino acid of the desired peptide sequence is first anchored to the polystyrene beads. The t-POC (or equivalent) protecting group of this amino acid is then removed. The next amino acid (in the protected t-POC form) is added together with dicyclohexylcarbodiimide, the coupling agent. After formation of the peptide bond, excess reagents and dicyclohexylurea are washed away, which leaves the beads with the desired dipeptide product. Additional amino acids are linked by the same sequence of reactions. At the end of the synthesis, the peptide is released from the beads by adding HF, which cleaves the carboxyl ester anchor without disrupting peptide bonds. Protecting groups on potentially reactive side chains, such as that of lysine, are also removed at this time. This cycle of reactions can readily be automated.

If in vitro protein synthesis is not possible, practical, or economical (or desired or practical), then the following processes are preferred. The preferred method of protein synthesis from the encoding DNA prepared above is by culturing cells transformed with an expression vector containing this DNA and recovering the binding protein of interest or variant from the cell culture, all by known methods. See, e.g., Sambrook et al., supra. Either prokaryotic or eukaryotic cells (for example, if post-translational modifications are needed) and expression vectors appropriate to the cell and cell type may be used.

Alternatively, it is further envisioned that the precursor binding protein of interest and the candidate engineered binding domains of interest may be produced by homologous recombination, as provided for in WO 91/06667 published May 16, 1991. Briefly, this method involves transforming cells expressing endogenous binding protein with a construct (i.e., vector) comprising an amplifiable gene (such as dihydrofolate reductase [DHFR] or others discussed below) and at least one flanking region of a length of at least about 150 bp that is homologous with a DNA sequence at the locus of the coding region of the precursor gene to provide amplification of the precursor gene. The amplifiable gene must be at a site that does not interfere with expression of the precursor gene. The transformation is conducted such that the construct becomes homologously integrated into the genome of the primary cells to define an amplifiable region.

Primary cells comprising the construct are then selected for by means of the amplifiable gene or other marker present in the construct. The presence of the marker gene establishes the presence and integration of the construct into the host genome. No further selection of the primary cells need be made, since selection will be made in the second host. If desired, the occurrence of the homologous recombination event can be determined by employing PCR and either sequencing the resulting amplified DNA sequences or determining the appropriate length of the PCR fragment when DNA from correct homologous integrants is present and expanding only those cells containing such fragments. Also if desired, the selected cells may be amplified at this point by stressing the cells with the appropriate amplifying agent (such as methotrexate if the amplifiable gene is DHFR), so that multiple copies of the target gene are obtained. Preferably, however, the amplification step is not conducted until after the second transformation described below.

After the selection step, DNA portions of the genome, sufficiently large to include the entire amplifiable region, are isolated from the selected primary cells. Secondary mammalian expression host cells are then transformed with these genomic DNA portions and cloned, and clones are selected that contain the amplifiable region. The amplifiable region is then amplified by means of an amplifying agent if not already amplified in the primary cells. Finally, the secondary expression host cells now comprising multiple copies of the amplifiable region containing the binding protein of interest are grown so as to express the gene and produce the binding protein.

After synthesis, either in vivo, or in vitro, or otherwise, the protein is purified and isolated by means known in the art, for example by high-performance liquid chromatography. Alternatively, the methods used for protein purification employing engineered binding domains may also be used to purify these domains in the first place. For example, affinity chromatography, also used as a purification technique, is described above.

### Systems of the Invention

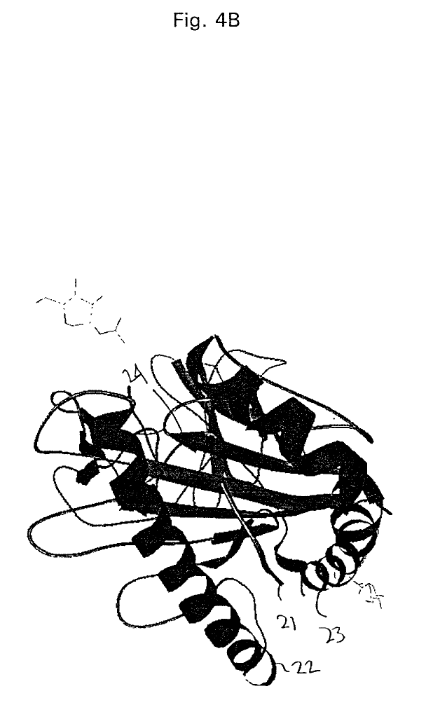

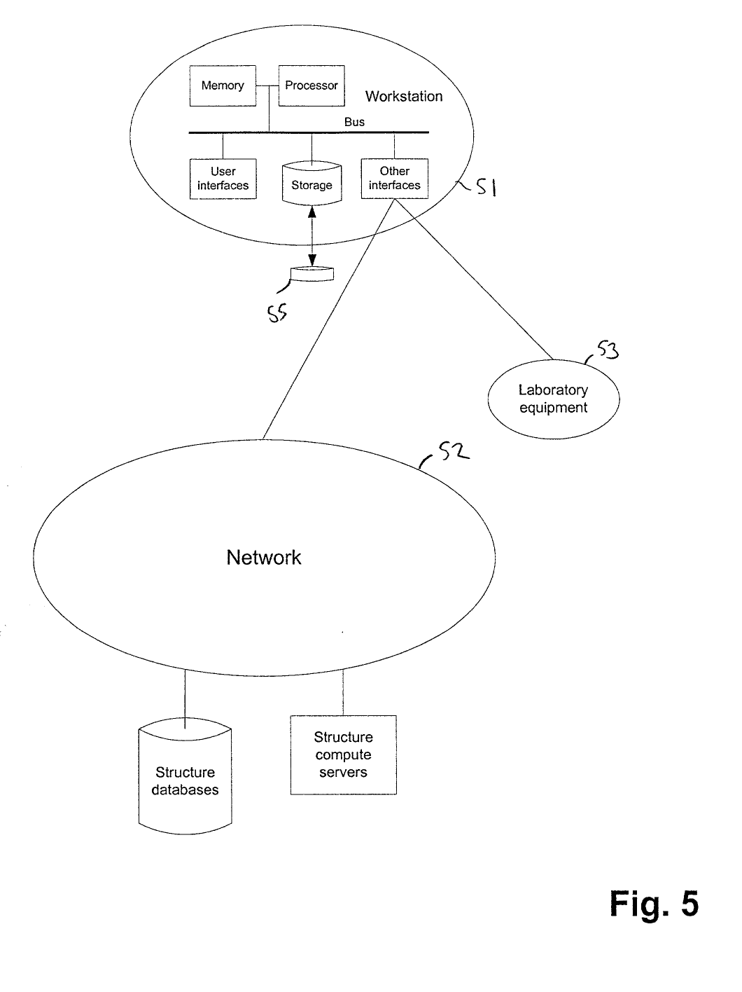

The present invention also includes systems and programs to carry out the methods of described herein. In one embodiment, the invention includes computers or computer systems for performing the CAMD design methods described above. FIG. 5 illustrates an exemplary such system which includes workstation-type computer 51, which may be a uni-processor or multiprocessor linked to network 52, which can be the Internet or an intranet. Computer 51 is illustrated here as a uni-processor configured in a standard manner with a processor, processor-accessible memory, permanent storage and various interfaces. Computer 51 is provided with user interfaces which provide for user command input and data output and, preferably, also for high-resolution graphical output of molecular models. The other interfaces can provide for, inter alia, communication over networks and, optionally, with local attached equipment.

Network 52 is typically the Internet which provides world-wide connection to resources from computer-assisted molecular modeling. For example, FIG. 6 illustrates a network-attached structure database, such as the Protein Data Bank for obtaining initial molecular structural data, and a network-attached structure computer server, such as the Swiss-Model which can perform some or all of the actual CAMD processes.

The programs to implement the above-described CAMD methods can be written in any standard computer language, such as C or C++. The source code can be translated into code executable by computer 51. The executable code can be loaded into the memory in order to cause the processor to carry out the CAMD methods described. As is well known in the computer arts, the executable code cooperates with data structures representing the proteins being designed, the precursor proteins, the alternative proteins, and the candidate proteins. In one embodiment, these data structures can be linked lists of structures representing the constituent amino acids, which in turn can simply be records with entries corresponding to the atoms making up the amino acids. These data structures also include necessary parameters, program control information, and so forth.

The code (source or executable) may be initially loaded into computer 51 from computer-readable media 55 onto which this code has been impressed. Computer-readable media 55 can be any type of optical or magnetic disk or other media, and can alternately represent download from a networked connected storage site.

In alternative embodiments, the systems of this invention may include the following additional features. It is preferable that the system provides molecular visualization software adapted to the display of biological macromolecules in order to permit a user to interactively examine models of the precursor binding domains and of alternative and candidate binding domains. Further, where this invention is practiced in connection with a laboratory for carrying out synthesis and assay methods, it is preferable that the CAMD systems be interfaced to the laboratory equipment so that to the extent possible the experimental processes can be automatically controlled. This may entail further software for the design of oligonucleotide primers, for determining experimental protocols for the assay and synthesis steps, for direct control of laboratory robots, micro-fluidics devices, and the like. Additionally, where the present invention is practiced for the large scale design and synthesis of numbers of engineered binding domains, software to integrate all methods of this invention, such as work-flow type software, is advantageous to create a binding domain “factory”.

### Uses of Engineered Binding Proteins

Generally, the engineered binding domains of the present invention can be used wherever binding proteins, for example, antibodies, have been used in the past, as well as for new uses used made possible for the first time by the present invention. Such new uses include, but are not limited to, applications of single domains, for example in engineered enzymatic catalysis, or in creating large numbers of binding domain, for example as protein libraries or protein chips, or so forth.

### Single Domains

In addition to use wherever uses single binding proteins have had in the past, the present invention provides for new uses of single binding proteins. For example, naturally-occurring precursor proteins of a wide range of biological activities can be engineered to provide new sequence-specific N- or C-terminal binding to the new target proteins. Possible uses of such engineered single domains are as diverse as the biological functions of the precursor proteins. These following illustrations are exemplary and not limiting.

### PDZ Domains

PDZ domains, which have natural roles in assembly of protein complexes, can be engineered according to the present invention and expressed intracellularly in order to cause the formation of new non-natural protein assemblies or complexes. Such new assemblies can be used as research tools and perhaps as therapeutic agents.

PDZ domains are firmly established as important in intracellular protein assembly and their mechanism of action established. PDZ domains are small (about 100 amino acids (and possible synthesized in vitro)) globular folds that mediate many protein-protein interactions. These domains were first recognized as conserved elements in the PSD-95 (postsynaptic density), Dgl (Discs-large), and ZO (zonula occludans) proteins; each of which is a member of the membrane-associated broad guanylate kinase (MAGUK) family, and each contains three copies of the PDZ element. It was subsequently demonstrated that a general function of PDZ domains is to promote clustering of proteins into complexes at the plasma membrane (Ponting. C. R et al. 1996. Bioassays 19:469-479). Examples of this include the clustering mediated by PSD-95 of Shaker-type K⁺ channels (Kim, E. et al. 1995. Nature 378:85-88). This type of interaction is believed to bring about the assembly of multi-protein signaling complexes to increase the efficacy of molecules involved in signaling and effector functions by concentrating them with respect to one another, as well as by localizing them to specific regions of the cell membrane (Craven. S E. and Bredt, D. S. 1998. Cell 93:495-498).

PDZ domains promote clustering through their binding to the C-terminal peptides of membrane proteins. The PDZ domain has been shown to recognize specifically the C-terminal motif (-x-Ser/Thr-x-Val-COO⁻), although peptides without this motif can also be bound (Songyang, Z. et al. 1997. Science 275:73-77). Several different proteins containing these ligand motifs can thus become clustered by association with a “scaffold” protein containing multiple PDZ domains. Further, the precise molecular details of PDZ-peptide recognition have been revealed by the crystal structures of PDZ domains, solved both in free and ligand-bound states (Doyle, D A. et al. 1996. Cell 85:1067-1076). PDZ are structurally related to PTB (phosphotyrosine binding) and PH (pleckstrin homology) domains (Harrison, S. C., 1996. Cell 86: 341-343) and share no sequence or structural relationship with TPR domains.

In view of such developments, the computational (and chemical) mutagenesis approach in this current method offers an efficient means to screen for desired mutated binding domains. In detail, the globular PDZ structure is composed mainly of a six-stranded β-sandwich, flanked by two α-helices. On its surface lies a peptide-binding groove defined by the edge of an antiparallel β-sheet and an α-helix. Binding of the ligand peptide in this groove positions the peptide to act as an additional antiparallel β-strand, thereby extending the sheet, a mode of binding referred to as β-augmentation (Harrison, S C. 1996. Cell 88:341-343). Unlike a classic protein-protein interaction that involves docking of relatively rigid complementary protein surfaces, the PDZ peptide interaction involves a ligand that is likely to be unstructured in its free form. This basic difference may impart an inherent flexibility to the range of potential PDZ-ligand recognition.

### TPR and Related Conditions

TPR and related binding domains engineered according to the present invention to bind to new targets may offer tools in cancer research and drug target screening. The TPR domains of Hop coordinate the functional cooperation of Hsp70/Hsc70 and Hsp90 in the folding of a number of proteins that feature prominently in cancer, including the androgen and estrogen receptors, and several proto-oncogenic protein kinases. Altering this binding by engineering or blocking it with potential drugs may interfere with the binding of Hsp70 or Hsp90 to Hop, thereby inhibiting the passage of client molecules to Hsp90 and changing the levels of various proto-oncogenic products. Similarly, blocking the binding of Hsp90 to other TPR partner proteins, such as certain immunophilins, might also reduce the functional levels of Hsp90 substrates.

In more detail, TPR domains depend on the target motif EEVD and binding to the terminal carboxyl group of a C-terminal region. The adaptor protein Hop mediates the association of the molecular chaperones Hsp70 and Hsp90. The TPR1 domain of Hop specifically recognizes the C-terminal heptapeptide of Hsp70 while the TRP2A domain binds the C-terminal pentapeptide of Hsp90, the sequences of both ending with the motif EEVD. The crystal structures of the TPR-peptide complexes show the peptides in an extended conformation, spanning a groove in the TPR domains (Scheufler, C. et al. 2000. Cell 101: 199-210). Peptide binding is mediated by electrostatic interactions with the EEVD motif, with the C-terminal aspartate acting as a two-carboxylate anchor, and by hydrophobic interactions with residues upstream of EEVD. The hydrophobic contacts with the peptide are critical for specificity. The structure shows that the TPR domains play an integral role in the ordered assembly of Hsp70-Hsp90 multi-chaperone complexes.

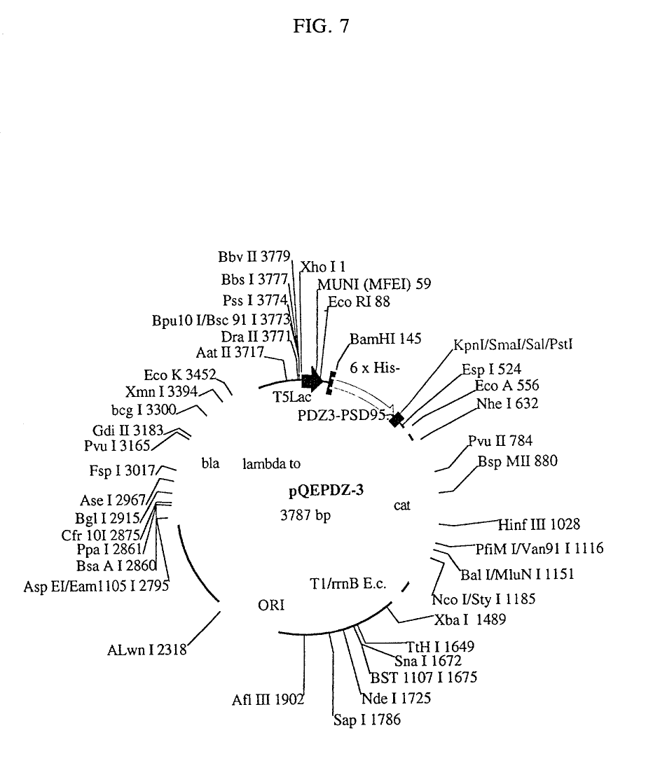

Furthermore, altering or blocking the binding to 14-3-3 domains, which are related to TPR domains, may offer new research or therapeutic tools related to manipulation of intracellular signaling pathways (Das et al., 1998. EMBO J. 17, 1192-1199). The 14-3-3 family of proteins have recently been identified as regulatory elements in intracellular signaling pathways: 14-3-3 proteins bind to oncogene and proto-oncogene products, including c-Raf-1, c-Bcr, and polyomavirus middle-T antigen; over expression of 14-3-3 activates Raf kinase in yeast and induces meiotic maturation in Xenopus oocyte. 14-3-3 domains form homodimers with nine α helices per monomer. A comparison of the TPR1-peptide complex with the recently determined structure of a 14-3-3 domain-peptide complex (Yaffe et al., 1997) suggested a different alignment (FIG. 7A). Upon fitting the 14-3-3 complex into the TPR1 complex in such a way as to maximize the fit of the two bound peptides, there is a striking super-positioning of functionality important residues of the two domains: five of the TPR1 helices align well with helices in the 14-3-3 domain (rmsd of 2.5 Å), and the five residues of TPR1 that form the two-carboxylate clamp can be superimposed with residues in 14-3-3 that are important for peptide binding (FIG. 7B). Four of these five residues in the 14-3-3 domain are responsible for all the electrostatic interactions with the bound peptide. It is apparent that large parts of the 14-3-3 domain do not have a structural equivalent in TPR domains. Interestingly, however, the portion of the 14-3-3 domain that cannot be aligned exclusively mediates contacts with the phosphate moiety but not with other regions of the bound peptide (Yaffe et al., 1997). This suggests that TPR domains and 14-3-3 domains are using the same architectural principles for peptide binding and that the functionality of 14-3-3 domains, i.e., the recognition of phosphorylated serine and threonine residues, is an additional feature that resides in a unique structural element outside the core common to both domain architectures that is used for sequence-specific peptide binding.

### Proline-Specific Peptidases

Engineering proline-specific peptidases may lead to peptidases of altered substrate specificity as well as providing candidate domains for binding to N-terminals of peptides. These enzymes bind to the terminal amino group of a protein and cleave a peptide bond that is either following or preceding a proline residue. The proline-specific aminopeptidases may be engineered using this invention to target different specific sequences at the N-terminal region of a protein to achieve specific binding and cleavage of the amide bond differently from the natural enzyme. By also engineering the catalytic site, the aminopeptidase activity may be reduced or eliminated, leaving only a specific binding function to a specific N-terminal peptide of a protein.

Proline-specific peptidases have been described in a wide variety of organisms and specifically cleave either the amide bond after a proline residue or the amide bond that precedes it (Yaron, A. & Naider, F. 1993. Crit. Rev. Biochem. Mol. Biol. 28, 31-81). Proline aminopeptidases specifically release the N-terminal residue from a peptide where the pen-ultimate residue is proline. These enzymes have significant sequence homology with (i) some other amino peptidases (e.g., methionine aminopeptidase, AMPM) and (ii) the Xaa-Pro dipeptidases, prolidases, that specifically cleave Xaa-Pro dipeptides. All these enzymes are activated in the presence of divalent metal ions, though the biologically active metal is not always certain. In vitro the prolyl peptidases are most active in the presence of Mn²⁻. Structural comparisons between AMPM and creatine amidinohydrolase (creatinase) and sequence comparisons between these proteins and aminopeptidase P (AMPP) and prolidase have suggested that the catalytic domains of all four enzymes have a common fold (Bazan, J. F. et al., 1994. Proc. Natl. Acad. Sci. USA 91, 2473-2477). The structures of a complex of AMPP with a dipeptide inhibitor and a low-pH inactive form offer a mechanism for peptide specific binding and hydrolysis (Wilce, M. et al., 1998. Proc. Natl. Acad. Sci. USA 95, 3472-3477).

### Class II MHC Proteins

Alternatively, specific binding to either terminal region of a protein may be obtained using the domain-engineering approach of the present invention, choosing a precursor binding domain where binding to the amine and carboxyl groups is not essential. Since Class II MHC proteins have this property, engineered Class II MHC binding domains thus can be used to more easily screen for specific interactions with other proteins.

Class II major histocompatibility complex (MHC) proteins are cell (B cells and macrophages) surface glycoproteins that bind peptides and present them to T cells as part of the mechanism for detecting and responding to foreign material in the body. The peptide-binding activity exhibits allele-specific preferences for particular side chains at some positions, usually at positions 1, 4, 6, and 9 (where the numbering refers to the normal MHC class II convention). Crystal structure of the well known human class II MHC protein HLA-DR1 in complex with the tight binding endogenous peptide A2 (103-117) shows the peptide-binding motif of HLA-DR1 has an aromatic residue at position+1, an arginine residue at position+2, and a small residue at position+6; these preferences can be understood in light of interactions observed in the peptide-MHC complex. Comparison of the structure with that of another MHC-peptide complex shows that completely different peptide sequences bind in essentially the same conformation and are accommodated with only minimal rearrangement of HLA-DR1 residues. Small conformational differences that are observed appear to be important in interactions with other proteins.

### Alteration of Cellular Protein Functions

The methods of this invention provides a practical way to select binding domains, such as PDZ domains, that will bind to the C termini of a wide range of proteins, raising interesting possibilities for conveniently modifying protein localization, metabolism, and/or function in vivo. Of course, experimental methods for directing protein localization are well established and include appending a cis-acting sequence such as a nuclear localization signal (NLS), or a lipid modification signal like the CAAX box. Alternatively, the addition of a dimerization domain (like a leucine zipper) can direct a protein to the locale of its cognate dimerization partner. A significant difference and advantage of this method, is that it offers the potential to alter the localization or activity of a native protein expressed from an unmodified gene by causing the intracellular expression of an engineering domain with an appended localization, modification, or metabolism signal. In another similar embodiment, an existing protein with a peptide binding domain may be altered by the methods of this invention to bind a new partner and thereby promote a formation of a non-natural protein assemblage with perhaps useful research or therapeutic possibilities.

The current interest in the development of “antisense” protein technology provides another possible application. This interest derives from the idea that it would be of general use to create recombinant proteins that bind specifically to particular cellular proteins, and thereby abolish or alter their functions in vivo. In contrast, binding domains engineered by the methods of this inventions and for example derived from the precursors described above, do not suffer from this particular limitation. It is clear how intracellular expression of engineered domain may act in a dominant negative fashion; more creative positive applications would seem inevitable. Proteins can be engineered to have multiple binding domains with different ligand specificities. The use of concatemers of different engineered domains may allow the creation of “chimeric” protein clusters with novel signaling or enzymatic properties.

### Enzynamatic Binding Sites

The active site of an enzyme is the region that binds the substrates (and the prosthetic group, if any) and contains the residues that directly participate in the making and breaking of bonds. These latter residues, known as the catalytic groups, are important in enzyme structure and binding specificity. Binding specificity is further dependent on the region spatially adjacent to the catalytic residues that further contribute to substrate binding, either specifically or non-specifically. Engineering such catalytic residues and/or the surrounding binding regions by the methods of the present invention can be used to develop enzyme variants to carry out a desired new protein-enzyme interactions, either new catalytic activities or new substrate specificities. This engineering is preferably done by the methods of this invention and in view of the following generalizations concerning enzymatic binding sites.

The active site takes up a relatively small part of the total volume of an enzyme. Most of the amino acid residues in an enzyme are not in contact with the substrate. Nearly all enzymes have more than 100 amino acid residues, which gives them a mass greater than 10 kd and a diameter of more than 25 Å. These other residues may provide stable scaffolding enabling the engineering of this invention. The active site is a three-dimensional entity formed by groups that usually come from different parts of the linear amino acid sequence. Indeed, residues far apart in the linear sequence may interact more strongly than adjacent residues in the amino acid sequence, as has already been seen for myoglobin and hemoglobin. It is often a cleft or crevice. In all enzymes of known structure, substrate molecules are bound to a cleft or crevice. Water is usually excluded unless it is a reactant. The nonpolar character of much of the cleft enhances the binding of substrate. However, the cleft may also contain polar residues. It creates a micro-environment in which certain of these residues acquire special properties essential for catalysis. The internal positions of these polar residues are biologically crucial exceptions to the general rule that polar residues are exposed to water.

Substrates are bound to enzymes by multiple weak attractions. Enzyme-substrate complexes usually have equilibrium dissociation constants that range from 10 mM to 10 nM, corresponding to free energies of interaction ranging from −3 to −12 kcal/mol, and are due to such reversible interactions as electrostatic bonds, hydrogen bonds, van der Waals forces, and hydrophobic interactions. Van der Waals forces become significant in binding only when numerous substrate atoms can simultaneously come close to many enzyme atoms. Hence, the enzyme and substrate should have complementary shapes. The directional character of hydrogen bonds between enzyme and substrate often enforces a high degree of specificity. The specificity of binding depends on the precisely defined arrangement of atoms in a binding region. To fit into the region, a substrate at least usually needs a matching shape. It is now evident that the shapes of the active sites of some enzymes may be markedly modified by the binding of substrate. The active sites of these enzymes have shapes that are complementary to that of the substrate only after the substrate is bound. This is a dynamic recognition process called induced fit.

For example, engineering the residues that promote substrate binding may by used to alter the substrate specificity of an enzyme. On the other hand, engineering the catalytic residues, which are often spatially distant, may alter reaction rates, the nature of the catalyzed reaction, or even substantially destroy catalytic action.

### Protein Libraries/Substrates

Biochemical studies of protein activity have usually focused on the analysis of single molecules. However, the rapid pace of discovery of new genes and gene products by large-scale genomics initiatives has necessitated alternative strategies for analyzing protein function. Molecular genetic screening techniques involving easily manipulated model organisms such as Escherichia coli, Saccharomyces cerevisiae, and Drosophila melanogaster have increased the number of proteins that can be analyzed in a single experiment. These studies tend to yield multiple subjects—peptides, proteins, or mutants—for additional rounds of experimentation on a molecule-by-molecule basis. Thus, the challenge is to develop high-throughput approaches to systematic and global protein analysis that place functionally unclassified proteins into a biological context.

The engineering methods of the current invention have the ability of rapidly and economically generating large numbers of specific binding domains, i.e., to a substantial fraction of the proteins expressed in a cell. These binding proteins clearly make available, for the first time, powerful new approaches in screening for large numbers target proteins with specific N- or C-terminal amino acid sequences. Several approaches for the systematic use of these binding proteins are possible, for example, as protein “libraries” or as protein arrays on substrates, also known as proteins “chips”.

Libraries of binding domains are one such high-throughput approach. In a library screen, a library of engineered binding domains is tested in batch, such as by contacting a sample of the library with a set of targets (peptides or proteins), followed by a selection step using downstream techniques, such as PCR, hybridization, or sequencing, is imposed to isolate candidate library members with desired binding properties, such as binding with one or more of the targets. Binding domain libraries can be maintained in several ways known in the art, several of which have been described above. For example, to name a few, domain libraries can be maintained for use in the various two-hybrid assays described above, such as libraries of vectors in yeast expressing binding domains fused with a transcriptional activator (or DNA binding domain). Domain libraries can also be maintained as RNA-domain fusions, or perhaps preferably, as phage display libraries. Libraries are generally maintained in recombinant organisms (also called “recombinant entities” herein), where phage and prokaryotic and eukaryotic cells are considered as “organisms.”

As compared to protein chips, the library approach offers certain advantages. Unlike arrays, libraries are not limited by the requirement that each individual component be observable, and so the upper size limit is orders of magnitude in excess of that of even the largest practical array. Indeed, currently available DNA arrays contain from 10³ to 10⁴ members, while libraries containing several-fold coverage of complex genomes with 10⁶ to 10⁷ members are routinely used. Conceivably, a single library may contain specific binding domains for substantially all expressed proteins in a cell. Further, the construction of libraries is less expensive, while the construction of arrays using currently available technology may require considerable investments, and efforts to ensure that the array meets quality standards may be significant.

Conversely, the array format is now an established method for analysis of nucleic acids, and the use of oligonucleotide arrays (“DNA chips”) for gene expression analysis has recently been reviewed (Debouck, C. & Goodfellow, P. N. DNA microarrays in drug discovery and development. Nat. Genet. 21(1 Suppl), 48-50 (1999)). Although in the past few years, this approach has been adapted for small protein studies, the present invention provides protein chips with radically increased capabilities. Conceivably, a single chip could contain binding domains for substantially all the proteins in a cell. Also, the binding domains placed on a protein chip may be selected to suit the aims of a particular experiment. Thus, a protein chip might contain binding domains for a substantial fraction of the signaling proteins, cell-cycle control proteins, phosphatases and kinases, or so forth in a cell, or of the proteins specific to organism development or its stages.

Using protein chips, elements may be screened with a selection step or alternatively, they may be screened in parallel assays. Typically, a sample of target proteins, for example extracted from a cell or organism, are first labeled, for example with fluorescent or biotin labels as known in the art, and then contacted with a protein chip in binding conditions. Unbound target is washed away and the label visualized. Positives are comparable and immediately identified by their position in the array. Thus the array format uses a precise, spatially ordered arrangement of elements that allows them to be examined side-by-side.

Protein arrays (“chips”) may generally be produced using and adapting materials and methods already developed for DNA chips. Robot and other automatic equipment may also be advantageously adapted to these new arrays. In one series of embodiments, substrates, which may be membranes, papers, plastics, or silica surfaces, are substantially flat, and are prepared and activated for covalent conjugation of the domains as is known in the art, perhaps with the attachment of linker moieties if necessary. (Alternately, the binding domain may be activated.) The binding domains are then contacted with the substrates under conjugating conditions to form the chips. Such binding-domain-substrate attachment is similar to that described above with respect to affinity chromatography. Binding domains can be placed on substrates by a variety of methods, such as micro-pipetting, ink-jet-type printing, or so forth.

In another series of embodiment, regions for individual binding domains may be defined by physical wells, depressions, or so forth, made in the following fashions. Protein chip substrates are preferably cast from master molds which have been stamped, milled, or etched using conventional micro fabrication or micro lithographic techniques. Preferably conventional micro lithographic techniques and materials are utilized in the production of the master molds (and the chip substrates themselves). Once a master mold has been produced, the master mold may then be used directly to mold the protein chips per se. Alternatively, secondary or tertiary molds may be cast from the master mold and the protein chips cast from these secondary or tertiary molds. The master mold may be made from any material that is suitable for micro fabrication or micro lithography, with silicon, glass, quartz, polyimides, and polymethylmethacrylate (Lucite) being preferred. For micro lithography, the preferred material is silicon wafers.

Once the appropriate master, secondary, or tertiary mold has been produced, the protein chip is cast. The protein chip may be cast in any solid support that is suitable for casting, including either porous or non-porous solid supports. Ceramics, amorphous silicon carbide, castable oxides that produce casts of SiO₂ when cured, polyimides, polymethylmethacrylates, and polystyrenes are preferred solid supports, with silicone elastomeric materials being most preferred. Of the silicone elastomeric materials, polydimethylsiloxane (PDMS) is the most preferred solid support. An advantage of silicone elastomeric materials is the ease with which they are removed from the mold due to their flexible nature. The substrate surface (or binding domains) may or may not additionally be activated to further secure their attachment.

Protein chips offer some significant advantages over protein libraries. If 1,000 elements are examined within an array format, a single experiment may be sufficient to yield 1,000 assay results, each directly comparable with the others. Moreover, the identity of each component in an array may be rapidly determined simply by its position. When screened as part of a library, only a subset of elements satisfying some criterion will be detected, and subsequent identification of isolated components typically requires significant downstream efforts. Library components are likely to compete with each other according to specific features of the screen, and this may lead to pleiotropic effects whereby the signal emanating from components with strong or dominant effects (including false positives) may obscure those whose action is more subtle. Thus, arrays have the advantage of being scalable, and their organized nature lends itself to high-throughput screening using robotic, imaging, or analytical methods.

### Diagnostic Kits

Engineered binding domains, including the engineered PDZ domains which recognize various short terminal sequences at C-terminus of a target protein, can be used for diagnostic purposes to detect, diagnose, or monitor diseases and/or disorders associated with the aberrant expression and/or activity of a protein of interest. The invention provides for the detection of aberrant expression of a protein of interest, comprising (a) assaying the expression of the protein of interest in cells or body fluid of an individual using one or more engineered binding domains specific to the protein of interest and (b) comparing the quantity of protein of interest with a standard protein quantity, whereby an increase or decrease in the assayed protein amount compared to the standard protein quantity is indicative of aberrant expression. The aberrant expression of a protein of interest may provides a diagnostic assay for diagnosing a disorder, whereby an increase or decrease in the assayed protein amount compared to the standard protein quantity is indicative of a particular disorder. With respect to cancer, the presence of a relatively high amount of protein of interest may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Engineered binding domains of the invention can be used to assay protein levels in a biological sample by first immobilizing engineered binding domains on a substrate. Various approaches are described above with respect to protein arrays. In fact, in a simplest embodiment, the diagnostic substrates here can be regarded as single “spot” protein chips. Proteins in biological samples can be labeled as is known in the art, for example by fluorescent labels, such as fluorescein and rhodamine, and biotin (Harlow et al. supra, 319-358). Binding domain-protein interaction can be observed by contacting the labeled sample to the immobilized binding domain, and then by detecting fluorescence after washing with proper buffer solutions.

The present invention provides kits that can be used in the above methods. In one embodiment, a kit comprises an engineered binding domain of the invention, preferably a purified binding domain, labeled with biotin and immobilized on a surface. Preferably, the kits of the present invention further comprise a control binding domain which does not bind the protein of interest, but may bind to a non-naturally occurring protein which appears in the kit and is introduced into a sample to be assayed for purposes of normalization. In another embodiment, the kits of the present invention also includes sample preparation reagents including labeling materials, washing buffers, and means for reading the label (if needed ). Preferably, the kit includes sufficient materials for several assays, for example by including a substrate with a number of spots of wells with engineered binding domain (and also a number of spots or wells with the control binding domain)

In one diagnostic configuration, a labeled test serum is mixed with a surface-bound binding domain obtained by the methods of the present invention. After binding with specific protein of interest and removing unbound serum components by washing, is detected by incubating the solid phase in the presence of a suitable fluorometric, luminescent or colorimetric substrate (Sigma, St. Louis, Mo.).

### Other Embodiments/Uses

In view of the above description, it will be readily apparent that this invention can engineer binding domains to both the N- and to the C-termini (or bivalent domains binding both termini) of proteins, indiscriminately. Engineering may include with the previously described CAMD techniques. Alternatively, the binding domains can engineered using the biotechnologies of combinatorial library synthesis, for example by directed mutagenesis, and library screening, for example by phase display.

### Examples

The following examples demonstrate, first, use of the methods of the present invention to redesign a PDZ domain to bind either to its natural target peptide sequence or to a new, non-natural target peptide sequence, and, second, binding assays of the wild-type and the redesigned domains with the natural and new targets. In particular, the yeast two-hybrid assays demonstrates that the redesigned PDZ domains bind with affinity and specificity to their proper targets in the midst of thousands intracellular proteins.

### The PDZ Domain

PDZ domains were chosen as an example of the present invention. These domains are found in many organisms where their main function is to target C-terminal sequences proteins, and thereby to localize their targets at specific sites within cells (Fanning & Anderson, 1999). For example, transport proteins with PDZ domains can transport the protein target of the PDZ domain to specific locations in the cell (Bunn et al., (1999). Mol Biol Cell 10(4), 819-32).

PDZ domains are small (about 100 amino acid residues), usually soluble, cytoplasmic, and with no disulfide bridges. Hence they provide a molecular framework that can be used both in vitro and in vivo. PDZ domains have been modified by random mutagenesis to new binding specificities (Schneider et al., 1999, Nature Biotech. 17, 170-175). Several spatial structures of PDZ domains in complex with their target peptide have been solved and are available in protein structure databases (see Table 1 above). In these structures, the target peptide is bound in an extended conformation inserted between a β-strand (elongating thus the β-sheet of the domain by one more β-strand) and an α-helix (Doyle et al., 1996) (FIGS. 2A-C).

Structure 1BE9, the PDZ3 domain of PSD-95 in complex with the C-terminal peptide sequence of Cript, the highest resolution X-ray structure available of a PDZ domain in a complex with its target, was used as the redesign precursor. The 1BE9 coordinate file describes the PDZ3 domain, contained in a fragment from residues 301 to 415 of PSD-95, and its bound target peptide, residues 5 to 9 of Cript (SEQ ID NO: 1; the first 4 residues are disordered). Using determined structures for other PDZ domains would have resulted in similarly good results. In fact, other PDZ domains that recognize 7 residues instead of 5 could have been used to obtain greater specificity.

### PDZ Domain Redesign

Defining the Space of Alternative PDZ Domains

The method (referred to herein as “Perla”) disclosed in U.S. patent application Ser. No. 09/387,741, filed Aug. 31, 1999, entitled “Computer-based method for macromolecular engineering and design” and by one or more of the inventors of the present application (incorporated by reference herein for all purposes), was used to model the binding of C-terminals of selected peptide sequences arranged within the binding region of the PDZ domain, in the manner in which the natural target is bound (FIGS. 2A-C), and then to evaluate changed residues surrounding the bound peptide in order to redesign the PDZ domain to recognize the new target sequences.

Five residue sequences were modeled in place of the initial target, while mutated PDZ domains were constructed and evaluated by Perla. Interacting residues forming the target binding pocket were selected for mutation through visual inspection of the three-dimensional structure. The following eleven variable residues were selected, as previously described, for mutation: L323, F325, N326, I327, I328, E331, H372, E373, A376, L379 and K380. These variable residues form a first structural layer around the target binding pocket. Three additional variable residues were also selected for mutation to improve the design of the binding domain for some target proteins: I338, L342 and I359.

Amino acids substitutions (mutations) at the variable residues were selected according to the amino-acid-type rules described above (Table 5). This limited the number of mutants modeled by Perla and shortened computation time. According to the amino-acid-type rules, hydrophobic residues were tried at buried positions, and polar ones at solvent-exposed positions. More limited choices were selected for certain residues spatially close to the new target sequence, e.g., only basic amino acids if close to an acidic amino acid (and conversely), or hydrophobic amino acids if close to non-polar residues, or polar amino acids if hydrogen bond were possible between the PDZ binding domain and the new target. Table 6 above lists the residues determined to be buried or solvent exposed, and the residues of the target peptide sequence contacted (as defined quantitatively by Perla).

Additional residues with non-fixed side chains (see above) were defined by Perla as those in spatial proximity to any of the 11 variable domain residues or the 5 target residues. Peturbation of the side chains of these additional non-fixed residues allowed redesigned domains to be more optimal in terms of both their interactions with the bound target and also with other residues of the PDZ domain precursor. The selected flexible residues were: I314, I316, R318, D332, E334, I336, F337, I338, S339, F340, I341, L342, P346, D348, L349, S350, L353, K355, D357, Q358, I359, V362, V365, L367, S371, Q374, I377, N381, V386, I388, Y397, R399, F400, E401. A number of Gly (3232, 324, 329, 330, 333, 335, 345 and 356) were also selected but have no side chains to be modeled. Modeled Ala side chains (the methyl group of which can rotate) were residues 347, 370, 375, 378 and 382. All other residue side chains were spatially fixed.

### Re-Design to Bind Natural Target

The C-terminal sequence of the natural target protein of the PDZ domain is listed above as SEQ ID NO: 1. Perla was first used to engineer residues of the PDZ domain to bind to natural target, as if the identity of amino acids in the binding interface were not known. All together, a sequence space of approximately 48×10⁶ sequences was considered, trying the following amino acid mutations:

TABLE 7
Results of Case 1

| Residue | Native PDZ | Alternative Amino | Best Results | Best Results |
|---|---|---|---|---|
| Position | Amino Acid | Acids Tried | of Run 1 | of Run 2 |
| 323 | Leu | A, V, I, L, F. | Leu | Phe |
| 325 | Phe | A, V, I, L, F | Leu | Phe |
| 326 | Asn | S, T, N, Q, D, E, | Lys | Lys |
| | | K, R | | |
| 327 | Ile | A, V, I, L, F | Ile, Leu | Ile, Leu |
| 328 | Ile | S, T, N, Q, D, E, | Lys, Thr | Thr |
| | | K, R | | |
| 331 | Glu | D, E | Glu | Glu |
| 372 | His | A, V, I, L, F | Ala, Leu | Ala, Ile, Leu |
| 373 | Glu | N, Q, D, E | Glu | Glu |
| 376 | Ala | A, V, I, L, F | A, V, L, F | A, V, L, F |
| 379 | Leu | A, V, L, F | Leu, Phe | Ile, Leu, Phe |
| 380 | Lys | N, Q, D, E, K, R | Lys, Arg | Lys, Arg |

At most interacting sites, conventional rules were followed: polar or charged amino acids at solvent-exposed positions and non-polar amino acids for buried positions. At certain sites inspection of the protein structures was necessary. Position 328 points towards the solvent-exposed protein surface but is partly buried between the bound peptide and the rest of the PDZ domain; the original Ile amino acid is thus quite optimal. Polar and charged amino acids were tried at this position in order to optimize the interaction with residues N6 and S8 from the bound protein. Position 331 is solvent-exposed and should interact with K5 from the bound protein; thus only acidic amino acids were considered. Position 372 and 376 are also solvent-exposed but could be buried by the bound peptide or protein; thus hydrophobic amino acids were tried at these positions.

Two computation runs were conducted with Perla, as explained above. Computation times were about 4 hours and 8 hours for the first and second runs, respectively (on an Silicon Graphics (SGI) computer). The second computation run allowed more flexibility of the side chain rotamer conformations. As a consequence larger amino acids were selected, e.g., Phe for positions 323 and 325. When choosing which sequences should be tried, special experimental attention was paid to larger residues, which provide for more interactions and better binding but could after all not fit within the binding pocket. If only few engineered binding domains were to be tried, computation runs that allowed less flexibility were used. Whenever more sequences would be tested, the proper strategy was to try sequences obtained in the various runs.

Experimental testing results are described below.

### Re-Design to Bind a Kinesin

The kinesin motor protein Eg5 was selected as a non-natural target protein, and the PDZ domain was engineered with Perla to bind specifically to a peptide sequence of this target. Eg5 is a cytoskeletal protein with a well-defined sub-cellular localization and a clear phenotypic effect upon inactivation. The C-terminus of murine Eg5 does not fulfill the general sequence consensus for sequences that bind PDZ domains, and therefore it is difficult to redesign a PDZ domain to bind Eg5. The C-terminal sequence of Eg5 is the following:

Thr Ser Ile Asn Leu (SEQ ID NO: 2)

See, Ferhat et al., (1998). J. of Neurosci. 18, 7822-7835.

The resign was carried out in two steps, first, focusing on the binding pocket as defined above, and then, including one additional residue position. A sequence space of approximately 72×10⁶ sequences was first considered, mutating the following amino acids:

TABLE 8
Case 2-A

| Residue | Native PDZ | Alternative Amino | Best Results | Best Results |
|---|---|---|---|---|
| Position | Amino Acid | Acids Tried | of Run 1 | of Run 2 |
| 323 | Leu | A, V, I, L, F | Leu | Leu |
| 325 | Phe | A, V, I, L, F | Ile, Leu | Leu |
| 326 | Asn | S, T, N, Q, D, E, | Lys | Lys |
| | | K, R | | |
| 327 | Ile | A, V, I, L, F | Ile, Leu, Phe | Ile, Phe |
| 328 | Ile | S, T, N, Q, D, E, | Lys | Lys |
| | | K, R | | |
| 331 | Glu | D, E | Glu | Glu |
| 372 | His | A, V, I, L, F | Leu | Ile, Leu |
| 373 | Glu | N, Q, D, E, K, R | Gln | Glu, Gln, |
| | | | | Lys |
| 376 | Ala | A, V, I, L, F | Ala, Val | Ala, Val |
| 379 | Leu | A, V, I, L, F | Ile, Leu, Phe | Ile, Leu, Phe |
| 380 | Lys | N, Q, D, E, K, R | Lys, Arg | Lys, Arg |

The choice of amino acids to try at the different positions differed from the previous design in the addition of basic residues at position 373. Visual inspection of the most promising redesigned PDZ domains (because of lower energy scores) suggested that residue 342 could interact with Asn8 from the bound protein, if mutated. Also, a hydrophobic cluster was observed to be formed between residues 372, 376, 379 plus Ile7 and Leu9 from the bound protein, and that residue 373 could be designed as non-polar amino acid to increase the size of this cluster. Hence, a second design with Perla was carried out to interatively improve the previous results. At some positions, only the best amino acids discovered in the previous design were tried. Polar and charged amino acids were tried at position 342, and position 326 (which are close in space) was completely redesigned. The size of the new sequence space was 4.8×10⁵ sequences:

TABLE 8
Case 2-B

| Residue | Native PDZ | Alternative Amino | Best Results | Best Results |
|---|---|---|---|---|
| Position | Amino Acid | Acids Tried | of Run 1 | of Run 2 |
| 323 | Leu | L, F | Leu | Leu |
| 325 | Phe | I, L, F | Leu | Leu |
| 326 | Asn | S, T, N, Q, D, E, | Thr, Lys | Thr, Lys |
| | | K, R | | |
| 327 | Ile | I, L, F | Ile, Leu | Ile, Phe |
| 328 | Ile | K | Lys | Lys |
| 331 | Glu | E | Glu | Glu |
| 342 | Leu | S, T, N, Q, D, E, | Lys, Arg | Lys, Arg |
| | | K, R | | |
| 372 | His | A, V, I, L, F | Leu | Leu |
| 373 | Glu | A, V, I, L, F, K, M | Ile | Ile |
| 376 | Ala | A, V | Ala, Val | Ala, Val |
| 379 | Leu | I, L, F | Ile, Leu, Phe | Ile, Leu, Phe |
| 380 | Lys | K, R | Lys, Arg | Lys, Arg |

The finally engineered K342 did not directly interact with Asn8 from the bound protein, but displaced the Asn8 side chain so that it formed a hydrogen bond with the redesigned T326. K328 was selected because it formed a salt-bridge with E401, and has no real interaction with the bound protein. I373 nicely complemented the hydrophobic cluster previously designed.

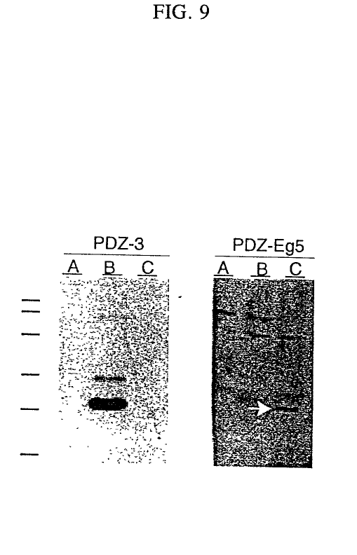

For experimental assay results see FIGS. 8 and 9.

### Selection of Sequences to be Tested Experimentally

Herein, for simplicity, only selected sequences of those that were proposed by the computer-based design method were experimentally tested. However, when many sequences can be tested experimentally, it is preferred to construct a defined library combining the selection of (best) amino acids obtained with the computer-based design method.

The sequences for testing were selected as follows. Residues (of a natural PDZ binding domain) were mutated when (i) the mutation was believed to improve the binding affinity and/or the selectivity (specificity) and (ii) the risk to disrupt or destabilize the binding domain was low compared to the expected gain in binding. Preferred mutations were selected, if possible, to conserve the chemical nature of the residue (conservative mutation). Non-conservative mutations were considered if required, for example at positions where key interactions with the target sequence could be established.

For the PDZ domain re-engineered to bind to its natural targets (having a Ser or Thr at positions−3 from the C-terminus), the two mutations N326K and I328T were selected for testing. His372 might have been mutated (either to Ala, Ile, or Leu) as proposed by the computer-based algorithm. However, examination of the various natural PDZ domains indicated that target sequences with a Ser or Thr at positions−3 (from the C-terminus) are recognized by PDZ domains having a conserved His residue. If His was included in the computational design, it was in fact preferred by the computer-based design method because a favorable hydrogen bond between His and the Thr side chain could be formed.

For the PDZ domain re-engineered to bind a kinesin, the mutations N326T, I328K, H372L, E373I, either A376 (no mutation) or A376V, and L379I were selected for testing. In this case His372 was mutated as the target sequence had no Thr/Ser at position−3. Thus, the H372L mutation should provide specific binding to the new target.

Additionally, all proteins had the mutations Q374E and I377A, on the surface of the α-helix. These positions are not part of the binding pocket, and the mutations were introduced to stabilize the α-helix and the protein. In principle, this increased the tolerance of the protein to manipulation of its binding site pocket.

### Redesigned PDZ Domain Assays

The redesigned PDZ domains were first cloned that assayed for binding to their new target.

Cloning the PDZ Domain

Redesign and assays were carried out using the third PDZ domain of the mouse PSD-95 protein, which spans amino acid 302 to 402 (Doyle et al, 1996). This fragment was amplified from a mouse brain cDNA library by PCR using primers PDZ-3U:

| | | |
|---|---|---|
| PDZ-3U: | TATGGATCCC TAGGGGAGGA AGATATTCCC CGGGAA | (SEQ ID NO: 3) |
| and PDZ-3L: | CGAGGTACCT CCCTTGGCCT CGAATCGGCT ATACTCTTCT GG | (SEQ ID NO: 4) |

(All nucleotide sequences are listed in the standard manner, from the 5′ end on the left to the 3′ end on the right.)

The amplified PCR fragment was cloned in the pQE30 expression vector (Qiagen) using the BamHI and KpnI sites to obtain the plasmid pQEPDZ-3 that encodes a 6Xhis-PDZ3 fusion (FIG. 7).

Peptide-GFP Fusions

To estimate the ability of the engineered PDZ domains to bind to the C-terminal regions of the target proteins, GFP (green fluorescent protein) derivatives were produced that contain the 8 last C-terminal amino acid residues of the target proteins. GFP-PEP contains the peptide that is known to be a target of the PDZ-3 domain of the PSD-95 protein (Doyle et al, 1996). GFP-EG5 contains the C-terminal octapeptide from the mouse Eg5 kinesin (Ferhat et al, 1999), namely:

Pro Leu His Thr Ser Ile Asn Leu (SEQ ID NO: 5)

GFP-PEP was prepared by cloning the annealed oligonucleotides pep-PDZ1:

| | | |
|---|---|---|
| pep-PDZ1: | GATCTACTAA AAACTATAAG GGAACCAGCG TATAATGA | (SEQ ID NO: 6) |
| and pep-PDZ2: | AGCTTCATTA TACGCGGTTC CCTTATAGTT TTTAGTA | (SEQ ID NO: 7) |

between the BgIII and HindIII sites of plasmid pEGPPC-1 (Clontech).

GFP-EG5 was produced by cloning the annealed oligonucleotides Eg5-1:

| | | |
|---|---|---|
| Eg5-1: | GATCTCCGCT TCACACCTCC ATAAACCTCT AGTAA | (SEQ ID NO: 8) |
| and Eg5-2 | AGCTTTACTA GAGGTTTATG GAGGTGTGAA GCGGA | (SEQ ID NO: 9) |

between the BgIII and HindIII sites of plasmid pEGPPC-1 (Clontech).

Mutant Constructs

A pQEPDZ-3 plasmid that had HinDII or PvuII restriction sites as unique sites within the pPDZ-cassette was first constructed. Nucleosides 243 and 246 of the PDZ domain coding sequence were changed to C in order to obtain a unique NgoMIV restriction site (without changing the sequence of the encoded protein).

To obtain PDZ Eg5A, two cloning steps were carried out. First, the annealed oligonucleotides Eg5-1L:

| | |
|---|---|
| Eg5-1L: | |
| CTGGACCCCC AGCAAGGATG AAGGAGATGA AGATGCCTTC ACCGTCCTCG CCGCCCTTAA TGGTGAAGCC CAGGCCG | (SEQ ID NO: 10) |
| and Eg5-1U: | |
| CTGGGCTTCA CCATTAAGGG CGGCGAGGAC GGTGAAGGCA TCTTCATCTC CTTCATCCTT GCTGGGGGT CCAG | (SEQ ID NO: 11) |

were inserted between the BstXI and PvuII sites of the pPDZ-cassette. Second, the annealed oligonucleotides Eg5A-2L:

| | | |
|---|---|---|
| Eg5 A-2L: | CCGGCGTTCT TGATGGCAGC GCAGCCTCAA TTAGACTGGC ATTGCGCAGG TC | (SEQ ID NO: 12) |
| and Eg5A-2U: | GACCTGCGCA ATGCCAGTCT AATTGAGGCT GTCGCTGCCA TCAAGAACG | (SEQ ID NO: 13) |

were inserted between the HinDII and NgoIV sites of the resulting plasmid.

PDZ Eg5B was obtained by cloning the annealed oligonucleotides Eg5-1L and Eg5-1U between the BstXI and PvuII sites of the PDZ cassette and cloning the annealed oligonucleotides Eg5B-2L:

| | | |
|---|---|---|
| Eg5B-2L: | CCGGCGTTCT TGATGGCAGC GGCAGCCTCA ATTAGACTGG CATTGCGCAG GTC | (SEQ ID NO: 14) |
| and Eg5B-2U: | GACCTGCGCA ATGCCAGTCT AATTGAGGCT GCCGCTGCCA TCAAGAACG | (SEQ ID NO: 15) |

between the HinDII and NgoIV sites of the resulting plasmid.

The PDZ (PDZ*) mutant with affinity to the wild type peptide was obtained cloning the annealed oligonucleotides PDZ-WT1L:

| | |
|---|---|
| PDZ-WTIL: | |
| CTGGACCCCC AGCAAGGATG AAGGAGATGA AGATGCCTTC ACCGTCCTCG CCACCGGTAA TCTTGAAGCC CAGGCCG | (SEQ ID NO: 16) |
| and PDZ-WT1U: | |
| CTGGGCTTCA AGATTACCGG TGGCGAGGAC GGTGAAGGCA TCTTCATCTC CTTCATCCTT GCTGGGGGTC CAG | (SEQ ID NO: 17) |

between the BstXI and PvuII sites of the PDZ cassette and cloning the annealed oligonucleotides Second, the annealed oligonucleotides PDZ-WT2L:

| | | |
|---|---|---|
| PDZ-WT2L: | CCGGCGTTCT TCAGGGCAGC GGCAGCCTCT TCATGACTGG CATTGCGCAG GTC | (SEQ ID NO: 18) |
| and PDZ-WT2U: | GACCTGCGCA ATGCCAGTCA TGAAGAGGCT GCCGCTGCCC TGAAGAACG | (SEQ ID NO: 19) |

were inserted between the HinDII and NgoIV sites of the resulting plasmid.

### Two-Hybrid Assays

The two parts of the yeast two-hybrid system were prepared as follows. On one hand, either of the GFP derivatives carrying the C-terminal protein sequences (GFP-pep and GFP-Eg5) were cloned between the NcoI and BamHI sites the pGBKT7 plasmid (Clontech) to obtain a fusion with the DNA binding domain of Gal4. On the other hand, the modified PDZ domains were cloned between the EcoRI and XhoI sites of pGADT7 (Clontech) to obtain a fusion with the activation domain of Gal4. The resulting plasmids were introduced in the yeast strain J569a using the method described by Agatep et al, 1998. Yeast cells were plated in SD lacking Trp and Leu, and then stricken in selective SD medium lacking Trp, Leu, His, and Adenine. Cells only grow in this selective medium if there is an interaction between the expression products of the two plasmids.

The three GFP derivatives, GFP-PEP; GFP-Eg5 and the original GFP fused to the binding domain of Gal4 were tested in all possible pair-wise combinations with the redesigned PGADT7-PDZEg5B and the original PDZ domains fused to the activation domain. Yeast cells transfected with only one of the two plasmids were also tested as controls.

FIG. 8 illustrates the results. Out of all these combinations only two allow viability of the transfected yeast cells plated in selective medium. The first combination is PGADT7-PDZ/EGF-PEP which shows the interaction of the unmodified PDZ with its natural target, thus providing a positive control for this experiment. The second combination that gives a positive result is GFP-Eg5/PGADT7-PDZEg5B. This result indicates that the re-designed PDZ interacts with the GFP-Eg5 fusion. Taken altogether, these observations show that the re-designed PDZ domain efficiently interacts with the Eg5 peptide.

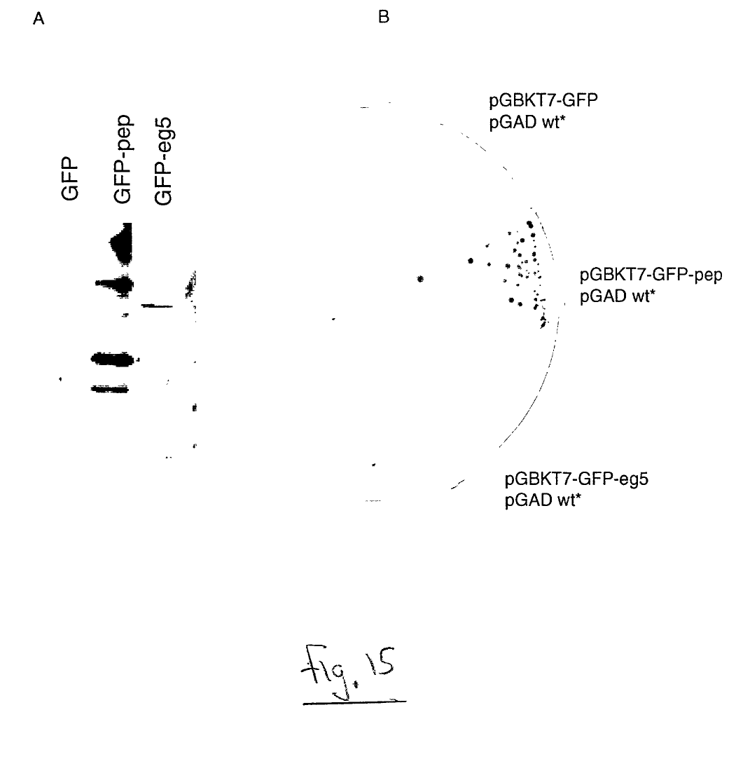

A further yeast two-hybrid system was constructed in a similar manner in order to assay the binding of PDZ-Wt*, the PDZ domain redesigned to bind to its natural target peptide sequence (referred to herein as “pep”). Panel B of FIG. 15 illustrates the results. Here, growth of viable cells correctly resulted only where PDZ-Wt* fused to the activation domain of gal4 was paired with GFP-pep fused to the binding domain of GAL4. No growth occurred with GFP alone of with GFP-Eg5 fused to the GAL4 binding domain.

### Affinity Chromatography Assays

To carry out the affinity chromatography assay, bacterially-expressed PDZ domains and EGFP derivatives were produced and fused to the different target peptides. The original and modified PDZ domains were expressed in E. coli XL1-Blue as fusions with 6XHis. The EGFP-peptides fusions were expressed in E. coli BL21 as described in Sambrook et al, 1989, Molecular Cloning: A Laboratory Manual, 2nd Ed., Vol 3. The bacterially expressed PDZ domains were then bound to nickel resin (Ni-NTA agarose, Quiagen) as described by the manufacturer. The resin-bound PDZ domains were then incubated with E.coli extracts made from bacteria expressing each of the target peptide-GFP fusions and washed four times with 50 mM NaH₂PO₄, 300 mM NaCl; 20 mM Imidazole; 0,1% of TritonX-100 to remove any unbound proteins. The bound proteins were eluted using 50 mM NaH₂PO₄, 300 mM NaCl; 200 mM Imidazole and analyzed by Western blot using antibodies against GFP to determine the presence of EGFP fused to the target peptide.

FIG. 9 illustrates the results of the chromatography. When the original PDZ was immobilized on a solid phase it was not able to bind either GFP or the GFP-Eg5 fusion, but it could very efficiently bind GFP fused to its target C-terminal peptide. This showed that these two molecules interacted in vitro and at the same time showed that affinity purification assay can be used to reveal such an interaction, as well as to purify, or enrich, the target protein. When the PDZ that had been designed to recognize the Eg5 C-terminal peptide was immobilized in the solid phase it was not able to bind GFP or its original target peptide, but it did bind the fusion protein that contained GFP and the Eg5 C-terminal peptide. This observation provided further evidence substantiating the conclusion that the re-designed PDZ domain indeed efficiently recognized the peptide with which it was intended to interact.

The same experiments were performed with the PDZ-Wt* and are illustrated in panel A of FIG. 15. Here, PDZ-Wt*, bound to nickel resin as above, could bind only GFP fused to its natural target pep. (The topmost band is residual anti-GFP antibody, while the bottom two bands are GFP-pep fusions one of which is partially truncated.) This redesigned domain did not bind either GFP alone or GFP fused to Eg5.

### Micro-Calorimetric Assay

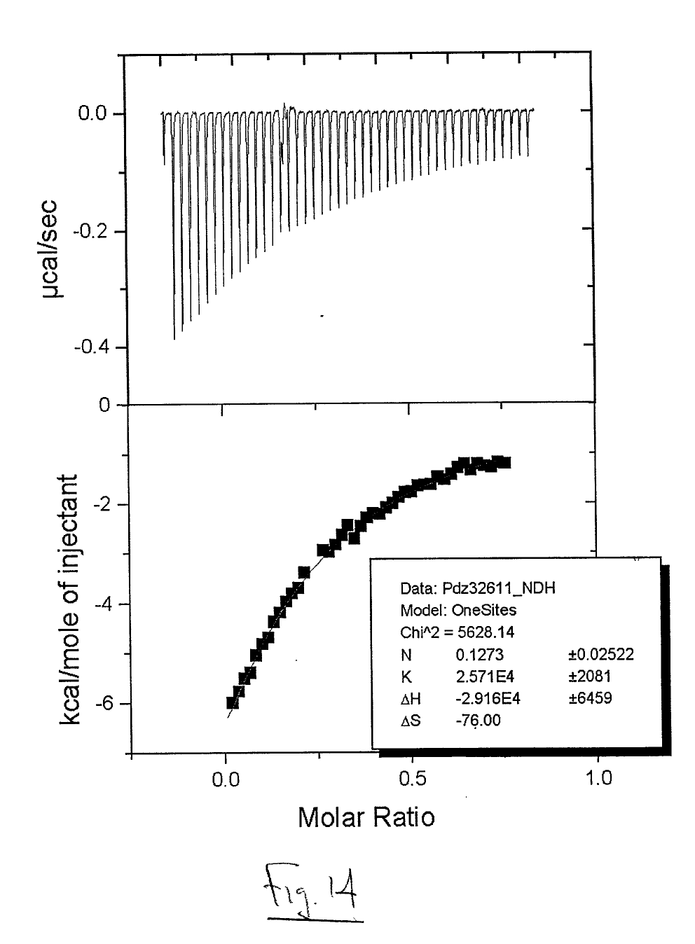

FIG. 14 illustrates the routine use of micro-calorimetry to determine the binding parameters of the wild-type PDZ (PDZ3 of PSD095) domain with its natural target (Biotin-GG-KQTSV). This figure present the output of an automated micro-calorimetry experiment returning the stoichiometry constant (n), the affinity constant (K), the enthalpy of binding (H), and the entropy of binding (S). Here, it is measured that the affinity constant is 2.57×10⁴ M⁻¹ (the corresponding dissociation constant is 39 μM).

### Expression in Mammalian Cells

Redesigned domains were tested for correct expression and function in mammalian cells. The fragment BamHI-NheI from the plasmid pQEPDZeg5B containing the sequence of the PDZ mutant was cloned in the BamH/XbaI site of pEGPF-C1 (Clontech). The resulting protein is a carboxy fusion of PDZ domain to EGFP.

NIH 3T3, mouse embryo cells, were transiently transfected with 10 μg of plasmid DNA by electroporation using a Multiporator (Eppendorf). The cell concentration was set to b 1×10⁶ cells/ml. Electroporation was carried out as described by the manufacturer (480 V, 100 μs, 1 pulse). After electroporation, the cells were cultured for 48 h prior to microscopy analysis.

For immunofluorescence microscopy, cells were fixed at room temperature for min with 4% of paraformaldehyde (PFA) in PBS (phosphate buffered saline), washed twice with PBS and fixed with cold methanol for min at −20 C. The methanol was removed and the samples were rehydrated with PBS containing 0.03% TritonX-100 (PBST) for 5 min. After re-hydration cells were treated for 15 min with PBS containing 25 mM Gly. The cells were then incubated with an anti-αTubulin antibody (N356 Amersham) diluted 1:1000 in PBST for 1 h at room temperature. After two washes with PBSB the cells were incubated with an anti-mouse IgG antibody diluted 1:1000 in PBST(Alexa™-594 Goat, Molecular Probes). After three washes with PBST the cells were mounted in vectashield (Vector Laboratories).

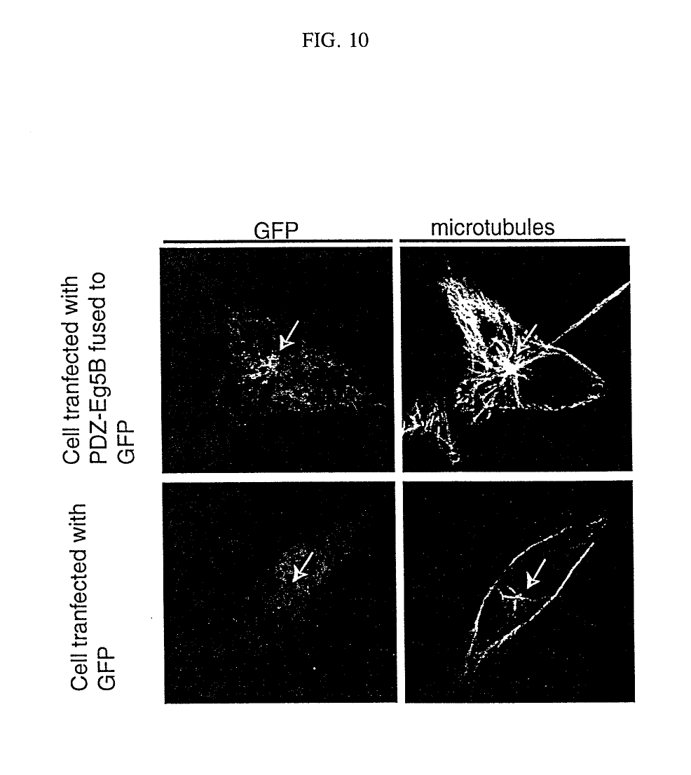

Cells were observed with a Leica confocal Microscope using a 40×oil immersion objective. FIG. 10 illustrated sub-cellular localization of a protein fusion made of GFP and a PDZ domain that had been engineered to recognize the centrosome-associated protein Eg5. The redesigned PDZ domain that recognized the C-terminus of Eg5 (PDZ-Eg5B) fused to GFP can be seen to accumulate around the microtubule organizing center where Eg5 is located (Cell 83:1159-1169 (1995); J Neurosciences 18:7822-7835 (1998)).

The invention described and claimed herein is not to be limited in scope by the preferred embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

A number of references are cited herein, the entire disclosures of which are incorporated herein, in their entirety, by reference for all purposes. Further, none of these references, regardless of how characterized above, is admitted as prior to the invention of the subject matter claimed herein.

What is claimed is:

1. A method of engineering one or more binding macromolecules to adequately bind to a selected target macromolecule, wherein macromolecules comprise linear polymers of monomers, said method comprising:
providing, as a first candidate binding macromolecule, a precursor macromolecule which binds to one or more terminal portions of an initial target macromolecule,
deriving alternative candidate binding macromolecules by replacing one or more monomers of a current candidate with new monomers, wherein the new monomers are selected by rational engineering methods so that the alternative candidates are predicted to bind with one or more terminal portions of the selected target macromolecule, and wherein the rational engineering methods depend on
(i) concerning the selected target macromolecule, input data comprising representations of one or more of its terminal monomer sequences, and
(ii) concerning the precursor macromolecule, input data comprising representations of its monomer sequence and the monomer sequences of one or more of the terminal portions of the initial target macromolecule bound by the precursor,
screening the alternative candidates for new candidates with improved estimated binding to terminal portions of the selected target, wherein the binding is estimated by rational methods in dependence on the input data, and
repeating, if necessary, the steps of deriving and screening until the estimated binding of one or more candidates is adequate, whereby one or more candidate macromolecules are engineered to bind to one or more terminal portions of the selected target macromolecule.

2. The method of claim 1 wherein the input data concerning the precursor macromolecule further comprises representations of its three-dimensional (3D) structure.

3. The method of claim 1 wherein the input data concerning the selected target macromolecule consists essentially of representations of one or more of its terminal monomer sequences.

4. The method of claim 1 further comprising the step of synthesizing one or more of the candidate binding macromolecules having adequate evaluated binding.

5. The method of claim 4 further comprising:
measuring the actual binding of the synthesized candidate macromolecules to the selected target macromolecule, and
repeating, if necessary, the steps of deriving, screening, and repeating and the further steps of synthesizing and measuring, until the measured actual binding of the synthesized candidates is adequate.

6. The method of claim 1 wherein binding is adequate if the dissociation constant (K_{d}) of a synthesized candidate macromolecule from the selected target macromolecule is less than a preselected maximum value.

7. The method of claim 6 wherein the maximum value is less than approximately 1 mM, or less than approximately 100 μM.

8. The method of claim 1 wherein the rational engineering or estimation methods comprise one or more computer-assisted molecular design (CAMD) methods.

9. The method of claim 1 wherein the steps of deriving and screening are repeated at least twice, wherein the rational engineering or estimating methods used in the later repetitions comprise more accurate methods, and wherein the rational engineering or estimating methods used in the earlier repetitions comprise less accurate methods.

10. The method of claim 1 wherein the monomers are amino acids, and wherein the macromolecules are peptides or polypeptides.

11. A method of engineering one or more binding polypeptides to adequately bind to a selected target polypeptide comprising:
providing, as a first candidate binding polypeptide, a precursor polypeptide which binds to one or more terminal peptide sequences of an initial target polypeptide,
deriving alternative candidate binding polypeptides by replacing one or more amino acid residues of a current candidate with new residues, wherein the new residues are selected by rational engineering methods so that the alternative candidates are predicted to bind with one or more terminal peptide sequences of the selected target polypeptide, and wherein the rational engineering methods depend on
(i) concerning the selected target polypeptide, input data comprising representations of one or more of its terminal peptide sequences, and
(ii) concerning the precursor polypeptide, input data comprising representations of its amino acid sequence, of the amino acid sequences of one or more of the terminal peptide sequences of the initial target polypeptide bound by the precursor,
screening the alternative candidates for new candidates with improved estimated binding to terminal peptide sequences of the selected target, wherein the binding is estimated by rational methods in dependence on the input data, and
repeating, if necessary, the steps of deriving and screening until the estimated binding of one or more candidates is adequate, whereby one or more candidate polypeptides are engineered to bind to one or more terminal peptide sequences of the selected target polypeptide.

12. The method of claim 11 wherein the precursor polypeptide comprises one or more PDZ-type domains, or one or more TPR-type domains, or one or more proline-specific-peptidase-type domains, or one or more class-II-MHC-protein-type domains.

13. The method of claim 11 wherein polypeptides comprise peptides having peptide sequences with lengths of less than approximately 20, or 15, or 10, or 5 residues.

14. The method of claim 13 wherein the one or more terminal peptide sequences of the selected target polypeptide comprise either its N-terminal or its C-terminal peptide sequence, or both.

15. The method of claim 11 wherein the input data concerning the precursor polypeptide further comprises representations of its three-dimensional (3D) structure.

16. The method of claim 11 wherein the input data concerning the selected target polypeptide consists essentially of representations of one or more of its terminal peptide sequences.

17. The method of claim 11 further comprising the step of synthesizing one or more of the candidate binding polypeptides having adequate evaluated binding.

18. The method of claim 17 wherein the step of synthesizing further comprises expression by recombinant genetic engineering methods or synthesis by chemical means.

19. The method of claim 17 wherein the step of synthesizing further comprises synthesizing one or more candidate binding polypeptides fused to one or more other polypeptides.

20. The method of claim 19 wherein the one or more other polypeptides occur in a cell of an organism.

21. The method of claim 19 wherein the one or more other polypeptides comprise green fluorescent protein.

22. The method of claim 17 further comprising:
measuring the actual binding of the synthesized candidate polypeptides to the selected target polypeptide, and
repeating, if necessary, the steps of deriving, screening, and repeating and the further steps of synthesizing, and measuring, until the measured actual binding of the synthesized candidates is adequate.

23. The method of claim 22 wherein the step of measuring further comprises performing affinity chromatography, or biosensor analysis, or micro-calorimetry.

24. The method of claim 22 wherein the step of measuring further comprises performing a yeast two-hybrid assay, or a phage display assay, or RNA-protein fusions.

25. The method of claim 22 further comprising the step of measuring binding specificity by measuring the actual binding of the synthesized candidate polypeptides to a plurality of polypeptides different from the selected target polypeptide.

26. The method of claim 11 wherein binding is adequate if the dissociation constant (K_{d}) of a synthesized candidate polypeptide from the selected target polypeptide is less than a preselected maximum value.

27. The method of claim 26 wherein the maximum value is less than approximately 1 mM, or less than approximately 100 μM.

28. The method of claim 11 wherein the rational engineering or estimating methods for polypeptides further comprise methods based on by a priori chemical or physical principles, or on rules derived from empirical knowledge, or on knowledge in the art.

29. The method of claim 28 further comprising storing information relating to previously engineered binding polypeptides to supplement the empirical knowledge or the knowledge in the art.

30. The method of claim 28 wherein the rational engineering or estimating methods based on principles further comprise one or more computer-assisted molecular design (CAMD) methods for polypeptides.

31. The method of claim 30 wherein the CAMD methods for polypeptides comprise methods which approximate side-chain conformations by rotamers from a rotamer-library, and which approximate polypeptide backbone conformations by an inverse-folding approach in dependence on a known 3D structure.

32. The method of claim 31 wherein the CAMD methods comprises a Perla method.

33. The method of claim 28 wherein the rational engineering or estimating methods based on rules further comprise rules derived from examples of sequence homology with known peptide-sequence-binding polypeptides, or derived from examples of polypeptides that bind to peptide sequences, or derived from examples of chimeric polypeptides formed from known peptide-sequence-binding polypeptides.

34. The method of claim 33 wherein the rules express peptide-sequence-binding specificities of peptide-sequence-binding polypeptides, or wherein the rules express how peptide-sequence-binding specificities of polypeptides may be modified.

35. The method of claim 28 wherein the rational engineering or estimating methods based on common knowledge further comprise rules for classifying amino acids into types with similar physical and chemical properties.

36. The method of claim 11 wherein the steps of deriving and screening are repeated at least twice, wherein the rational engineering or estimating methods used in the later repetitions comprise more accurate methods, and wherein the rational engineering or estimating methods used in the earlier repetitions comprise less accurate methods.

37. The method of claim 11 wherein the precursor polypeptide binds to two or more terminal peptide sequences of the initial target polypeptide, and wherein the step of repeating, if necessary, repeats the steps of deriving and screening until the estimated binding of one or more candidates to two or more terminal peptide sequences of the selected target polypeptide is adequate.

38. The method of claim 37 wherein the two or more terminal peptide sequences of the selected target polypeptide comprise both the N-terminal and the C-terminal peptide sequences, whereby one or more candidate polypeptides are engineered to bind bivalently to the selected target polypeptide.

39. The method of claim 11 further comprising a step of performing methods of nuclear magnetic resonance spectroscopy or x-ray crystallography to obtain structural data concerning one or more candidates, and wherein the rational engineering and estimating methods further depend on input of this structural data.

40. A method of engineering one or more binding polypeptides to adequately bind to a selected target polypeptide, the method comprising:
providing, as a first candidate binding polypeptide, a precursor polypeptide which binds to one or more N-terminal peptide sequences of an initial target polypeptide, wherein a peptide sequence has a length of less than approximately 20, or 15, or 10, or 5 residues,
deriving alternative candidate binding polypeptides by replacing one or more amino acid residues of a current candidate with new residues,
screening the alternative candidates for new candidates with improved binding to N-terminal peptide sequences of the selected target polypeptide, and
repeating, if necessary, the steps of deriving and screening until the binding of one or more candidates is adequate.

41. The method of claim 40 wherein the precursor polypeptide comprises one or more PDZ-type domains, or one or more TPR-type domains, or one or more proline-specific-peptidase-type domains, or one or more class-II-MHC-protein-type domains.

42. The method of claim 40 wherein the steps of deriving and screening further comprise rational engineering or estimation methods for polypeptides based on by a priori chemical or physical principles, or on rules derived from empirical knowledge, or on knowledge in the art.

43. The method of claim 42 wherein the precursor polypeptide has a known three-dimensional (3D) structure.

44. The method of claim 40 wherein the steps of deriving and screening further comprise synthesizing one or more of the alternative candidate binding polypeptides.

45. A computer system for engineering one or more binding polypeptides from a selected precursor polypeptide, wherein the precursor polypeptide binds to one or more terminal peptide sequences of an initial target polypeptide, and wherein the binding polypeptides adequately bind to a selected target polypeptide, the system comprising:
a processor, and
a memory accessible to the processor, wherein the memory is configured with
(a) data for representing the precursor polypeptide, the initial target polypeptide, the selected target polypeptide, and further candidate polypeptides, and wherein
(i) the data for representing the selected target polypeptide comprises data representing one or more of its terminal peptide sequences, and
(ii) the data representing the precursor polypeptide comprises data representing its amino acid sequence and of the amino acid sequences of the one or more terminal peptide sequences of the initial target polypeptide bound by the precursor, and
(b) instructions for causing the processor, in dependence on the represented data, to perform the steps of
(i) rational engineering methods for deriving alternative candidate binding polypeptides by replacing one or more amino acid residues of a current candidate with new residues so that the alternative candidates are predicted to bind with one or more terminal peptide sequences of the selected target polypeptide,
(ii) rational binding-estimating methods for screening the alternative candidates for new candidates with improved estimated binding to terminal peptide sequences of the selected target, and
(iii) repeating, if necessary, the steps of rational engineering and estimating until the estimated binding of one or more candidates is adequate, whereby one or more candidate polypeptides are engineered to bind to one or more terminal peptide sequences of the selected target polypeptide.

46. The system of claim 45 wherein data represents polypeptides as comprising peptides having peptide sequences with lengths of less than approximately 20, or 15, or 10, or 5 residues.

47. The system of claim 45 wherein the data represents one or more terminal peptide sequences of the selected target polypeptide as comprising either its N-terminal or its C-terminal peptide sequence, or both.

48. The system of claim 45 wherein the data representing the precursor polypeptide further comprises data representing its three-dimensional (3D) structure, and wherein the data representing the selected target polypeptide consists essentially of data representing its one or more of its terminal peptide sequences.

49. The system of claim 45 wherein the selected precursor polypeptide binds to two or more terminal peptide sequences of the initial target polypeptide, and wherein the instructions for repeating, if necessary, cause the processor to repeat the steps of rational engineering and rational estimating until the estimated binding of one or more candidates to two or more terminal peptide sequences of the selected target polypeptide is adequate.

50. The method of claim 49 wherein the two or more terminal peptide sequences of the selected target polypeptide comprise both the N-terminal and the C-terminal peptide sequences, whereby one or more candidate polypeptides are engineered to bind bivalently to the selected target polypeptide.

51. The system of claim 45 wherein the instructions for causing the processor to perform the steps of rational engineering or of rational binding-estimating further comprise instructions for performing methods based on by a priori chemical or physical principles, or on rules derived from empirical knowledge, or on knowledge in the art.

52. The method of claim 51 wherein the methods based on principles further comprise one or more computer-assisted molecular design (CAMD) methods for polypeptides.

53. The method of claim 52 wherein the CAMD methods for polypeptides comprise methods which approximate side-chain conformations by rotamers from a rotamer-library, and which approximate polypeptide backbone conformations by an inverse-folding approach in dependence on a known 3D structure.

54. The method of claim 53 wherein the CAMD methods comprises a Perla method.

55. The system of claim 52 wherein the instructions for causing the processor to perform a CAMD method further comprise instructions for performing two or more CAMD methods of increasing accuracy.

56. A computer-readable medium with encoded instructions stored therein for causing a computer to perform the method of claim 45

57. A polypeptide for binding to a selected target polypeptide engineered according to the method of claim 11.

58. A vector for causing expression in a host cell of a polypeptide engineered for binding to a selected target polypeptide according to the method of claim 11.

59. A cell comprising a nucleic acid sequence encoding a polypeptide engineered for binding to a selected target polypeptide according to the method of claim 11.

60. The cell of claim 59 further comprising the engineered polypeptide.

61. The cell of claim 59 further comprising the engineered polypeptide fused to a partner polypeptide comprising a peptide or a polypeptide.

62. The cell of claim 61 wherein the partner polypeptide comprises a polypeptide sequence causing the localization of the fusion to a selected intracellular compartment.

63. The cell of claim 61 wherein the partner polypeptide comprises a polypeptide sequence causing degradation of the fusion.

64. The cell of claim 61 wherein the partner polypeptide comprises a label.

65. The cell of claim 64 wherein the label is green fluorescent protein.

66. A method for altering the function of a first cellular protein, which does not naturally bind to a second cellular protein, comprising:
providing a binding protein engineered according to the method of claim 11 for binding to the second cellular protein, and
expressing the binding protein fused to the first cellular protein so that first cellular protein as part of the fusion binds non-naturally to the second cellular protein.

67. A cell comprising a nucleic acid encoding a cellular protein altered according to claim 66.

68. A method for altering the function of a selected cellular protein, which naturally binds to an initial polypeptide, comprising:
engineering the selected cellular protein to bind to a new target polypeptide according to the method of claim 11, and
expressing the engineered selected cellular protein.

69. The method of claim 68 wherein the initial polypeptide is a part of a first cellular protein to which the selected cellular protein naturally binds, and wherein the new target polypeptide is part of a second cellular protein to which the selected cellular protein does not naturally bind.

70. The method of claim 68 wherein the selected cellular protein is an enzyme, and wherein the engineered selected protein has altered substrate specificity or enzymatic activity.

71. A cell comprising a cellular protein with function altered according to claim 69.

72. A method for assaying for one or more target polypeptides in a sample comprising:
contacting, in binding conditions, the sample with binding polypeptides, wherein one or more binding polypeptides are engineered by the method of claim 11 to bind to one or more of the target polypeptides, and
assaying for binding polypeptides bound to their respective target polypeptides, whereby the target polypeptides are assayed.

73. The method of claim 72 further comprising, before the step of contacting, a step of attaching one or more binding polypeptides to a substrate.

74. The method of claim 72 wherein the step of assaying further comprises performing affinity chromatography, or biosensor analysis, or nuclear magnetic resonance spectroscopy, or micro-calorimetry.

75. The method of claim 72 wherein the step of assaying further comprises performing a yeast two-hybrid assay, or a phage display assay, or RNA-protein fusion.

76. The method of claim 72 wherein the one or more target polypeptides is a single target polypeptide.

77. A method of determining the cellular localization of a target protein comprising:
providing a binding protein engineered by the method of claim 11 to bind to the target protein,
contacting the cell with the binding protein under binding conditions, and
assaying for the presence and location in the cell of the binding protein bound to the target protein.

78. The method of claim 77 wherein the binding protein is fused to a polypeptide label.

79. The method of claim 77 wherein the step of assaying further comprises performing an immuno-chemical method using antibodies to the binding protein.

80. The method of claim 77 wherein the step of contacting comprises expressing the binding protein in the cell.

81. A method for assaying for target proteins in a sample from an organism comprising:
contacting, in binding conditions, the sample with binding polypeptides, wherein the binding polypeptides bind to one or more terminal peptide sequences of a plurality of selected proteins expressed in the organism, and wherein the plurality of selected expressed proteins comprises more than 50 different proteins, and
assaying for binding polypeptides bound to their respective target proteins, whereby the target proteins are assayed.

82. The method of claim 81 further comprising a step of engineering the binding proteins to bind to the terminal peptide sequences of the selected plurality of proteins by the method of claim 11.

83. The method of claim 81 further comprising the step of expressing the binding proteins in members of one or more libraries of recombinant entities.

84. The method of claim 81 wherein the terminal peptide sequences are N-terminal sequences, or C-terminal sequences, or both, having lengths less than approximately 15 amino acids.

85. The method of claim 81 wherein the plurality of selected proteins comprises more than 500 or more than 5,000 different proteins.

86. The method of claim 81 wherein the plurality of selected proteins comprises less than 5,000 or less than 50,000 different proteins.

87. The method of claim 81 wherein the plurality of selected proteins comprises more than 0.5% of the proteins expressed in a cell of the organism.

88. The method of claim 81 wherein the plurality of selected proteins comprises less than 50% or less than 80% of the proteins expressed in a cell of the organism.

89. The method of claim 81 wherein the binding polypeptides are attached to one or more substrates.

90. A library comprising recombinant organisms expressing a plurality of binding polypeptides,
wherein each binding polypeptide binds to one or more terminal peptide sequences of each of a plurality of selected proteins expressed in an organism, and
wherein the plurality of selected expressed proteins comprises more than 50 different proteins.

91. The library of claim 90 wherein the recombinant organisms comprise phage particles.

92. The library of claim 90 wherein the recombinant organisms are comprise cells for use in a two-hybrid assay.

93. The library of claim 90 wherein the terminal peptide sequences are N-terminal sequences, or C-terminal sequences, or both, having lengths less than approximately 15 amino acids.

94. The library of claim 90 wherein the plurality of selected proteins comprises more than 500 different proteins.

95. The library of claim 90 wherein the plurality of selected proteins comprises more than 5,000 different proteins.

96. The library of claim 90 wherein the plurality of selected proteins comprises less than 5,000 different proteins.

97. The library of claim 90 wherein the plurality of selected proteins comprises less than 50,000 different proteins.

98. The library of claim 90 wherein the plurality of selected proteins comprises more than 0.5% of the proteins expressed in a cell of the organism.

99. The library of claim 90 wherein the plurality of selected proteins comprises less than 50% of the proteins expressed in a cell of the organism.

100. The library of claim 90 wherein the plurality of selected proteins comprises less than 80% of the proteins expressed in a cell of the organism.

101. The library of claim 90 wherein the binding proteins are engineered to bind to the terminal peptide sequences of the selected plurality of proteins by the method of claim 11.

102. A polypeptide array comprising:
a substrate with at least one surface, and
a plurality of binding polypeptides regularly arranged on the surface,
wherein each binding polypeptide binds to one or more terminal peptide sequences of each of a plurality of selected proteins expressed in an organism, and
wherein the plurality of selected expressed proteins comprises more than 50 different proteins.

103. The array of claim 102 wherein the binding polypeptides are covalently attached to the surface.

104. The array of claim 102 wherein the substrate comprises glass or plastic.

105. The array of claim 102 wherein the terminal peptide sequences are N-terminal sequences, or C-terminal sequences, or both, having lengths less than approximately 15 amino acids.

106. The array of claim 102 wherein the plurality of selected proteins comprises more than 500 different proteins.

107. The array of claim 102 wherein the plurality of selected proteins comprises more than 5,000 different proteins.

108. The array of claim 102 wherein the plurality of selected proteins comprises less than 5,000 different proteins.

109. The array of claim 102 wherein the plurality of selected proteins comprises less than 50,000 different proteins.

110. The array of claim 102 wherein the plurality of selected proteins comprises more than 0.5% of the proteins expressed in a cell of the organism.

111. The array of claim 102 wherein the plurality of selected proteins comprises less than 50% of the proteins expressed in a cell of the organism.

112. The array of claim 102 wherein the plurality of selected proteins comprises less than 80% of the proteins expressed in a cell of the organism.

113. The array of claim 102 wherein the binding polypeptides are engineered to bind to the terminal peptide sequences of the selected plurality of proteins by the method of claim 11.

114. A polypeptide-RNA-fusion array comprising:
a substrate with at least one surface, and
a plurality of binding-polypeptide-RNA fusions regularly arranged on the surface,
wherein each binding polypeptide of the fusions binds to one or more terminal peptide sequences of each of a plurality of selected proteins, and
wherein the RNAs of the fusions comprise sequences that encode for the corresponding fused binding polypeptides.

115. The array of claim 114 wherein the terminal peptide sequences are N-terminal sequences, or C-terminal sequences, or both, having lengths less than approximately 15 amino acids.

116. The array of claim 114 wherein the plurality of selected proteins comprises more than 500 different proteins.

117. The array of claim 114 wherein the plurality of selected proteins comprises less than 5,000 different proteins.

118. A method of purifying one or more selected proteins from a sample comprising:
providing one or more binding polypeptides that bind to one or more of the terminal peptide sequences of one or more selected proteins, wherein the binding polypeptides are engineered by the method of claim 11,
contacting the sample in binding conditions with the binding polypeptides so that selected proteins in the contacted sample are bound to the binding proteins,
washing the contacted sample in washing conditions so that unbound proteins are removed while bound selected proteins remain, and
eluting the washed sample in eluting conditions so that bound selected proteins are removed from the binding polypeptides, whereby the eluted selected proteins are purified from the sample.

119. The method of claim 118 wherein the step of providing further provides the binding polypeptides attached to a substrate.

120. The method of claim 118 wherein the steps of contacting, washing, and eluting are performed by the methods of affinity chromatography.

121. The method of claim 118 wherein at least two of the selected proteins are bound in a protein complex.

122. A method of engineering one or more binding macromolecules to adequately bind to a selected target macromolecule, wherein macromolecules comprise linear polymers of monomers, said method comprising:
providing, as a first candidate binding macromolecule, a precursor macromolecule which binds to one or more terminal portions of an initial target macromolecule,
deriving alternative candidate binding macromolecules by replacing one or more monomers of a current candidate with new monomers, wherein the new monomers are selected by rational engineering methods so that the alternative candidates are predicted to bind with one or more terminal portions of the selected target macromolecule, and wherein the rational engineering methods depend on
(i) concerning the selected target macromolecule, input data consisting essentially of representations of one or more of its terminal monomer sequences, and
(ii) concerning the precursor macromolecule, input data comprising representations of its monomer sequence, its three-dimensional (3D) structure, and the monomer sequences of one or more of the terminal portions of the initial target macromolecule bound by the precursor,
screening the alternative candidates for new candidates with improved estimated binding to terminal portions of the selected target, wherein the binding is estimated by rational methods comprising one or more computer-assisted molecular design (CAMD) methods in dependence on the input data, and
repeating, if necessary, the steps of deriving and screening until the estimated binding of one or more candidates is adequate, wherein binding is adequate if the dissociation constant (K_{d}) of a synthesized candidate polypeptide from the selected target polypeptide is less than approximately 100 μM, whereby one or more candidate macromolecules are engineered to bind to one or more terminal portions of the selected target macromolecule.

123. A method of engineering one or more binding polypeptides to adequately bind to a selected target polypeptide comprising:
providing, as a first candidate binding polypeptide, a precursor polypeptide which binds to one or more terminal peptide sequences of an initial target polypeptide, wherein the one or more terminal peptide sequences of an initial target polypeptide comprise either an N-terminal, or a C-terminal peptide sequence, or both, with lengths of less than approximately 15 residues,
deriving alternative candidate binding polypeptides by replacing one or more amino acid residues of a current candidate with new residues, wherein the new residues are selected by rational engineering methods so that the alternative candidates are predicted to bind with one or more terminal peptide sequences of the selected target polypeptide, wherein the one or more terminal peptide sequences of the selected target polypeptide comprise either an N-terminal, or a C-terminal peptide sequence, or both, with lengths of less than approximately 15 residues, and wherein the rational engineering methods depend on
(i) concerning the selected target polypeptide, input data consisting essentially of representations of one or more of its terminal peptide sequences, and
(ii) concerning the precursor polypeptide, input data comprising representations of its amino acid sequence, of its three-dimensional (3D) structure, and of the amino acid sequences of one or more of the terminal peptide sequences of the initial target polypeptide bound by the precursor,
screening the alternative candidates for new candidates with improved estimated binding to terminal peptide sequences of the selected target, wherein the binding is estimated by rational methods comprising one or more computer-assisted molecular design (CAMD) methods for polypeptides in dependence on the input data, and
repeating, if necessary, the steps of deriving and screening until the estimated binding of one or more candidates is adequate, wherein binding is adequate if the dissociation constant (K_{d}) of a synthesized candidate polypeptide from the selected target polypeptide is less than approximately 100 μM, whereby one or more candidate polypeptides are engineered to bind to one or more terminal peptide sequences of the selected target polypeptide.

124. The method of claim 123 wherein the CAMD methods for polypeptides comprise methods which approximate side-chain conformations by rotamers from a rotamer-library, and which approximate polypeptide backbone conformations by an inverse-folding approach in dependence on a known 3D structure.

125. The method of claim 123 wherein the rational engineering or estimating methods further comprise rules derived from examples of sequence homology with known peptide-sequence-binding polypeptides, or derived from examples of polypeptides that bind to peptide sequences, or derived from examples of chimeric polypeptides formed from known peptide-sequence-binding polypeptides.

126. The method of claim 123 wherein the terminal peptide sequences have lengths of less than approximately 10 residues.

127. A method of engineering one or more binding polypeptides to adequately bind to a selected target polypeptide comprising:
providing, as a first candidate binding polypeptide, a precursor polypeptide which binds to two or more terminal peptide sequences of an initial target polypeptide, wherein the two or more terminal peptide sequences of an initial target polypeptide comprise either N-terminal or C-terminal peptide sequences, or both, with lengths of less than approximately 15 residues,
deriving alternative candidate binding polypeptides by replacing one or more amino acid residues of a current candidate with new residues, wherein the new residues are selected by rational engineering methods so that the alternative candidates are predicted to bind with two or more terminal peptide sequences of the selected target polypeptide, wherein the two or more terminal peptide sequences of the selected target polypeptide comprise either N-terminal or C-terminal peptide sequences, or both, with lengths of less than approximately 15 residues, and wherein the rational engineering methods depend on
(i) concerning the selected target polypeptide, input data consisting essentially of representations of two or more of its terminal peptide sequences, and
(ii) concerning the precursor polypeptide, input data comprising representations of its amino acid sequence, of its three-dimensional (3D) structure, and of amino acid sequences of two or more of the terminal peptide sequences of the initial target polypeptide bound by the precursor,
screening the alternative candidates for new candidates with improved estimated binding to terminal peptide sequences of the selected target, wherein the binding is estimated by rational methods comprising one or more computer-assisted molecular design (CAMD) methods for polypeptides in dependence on the input data, and
repeating, if necessary, the steps of deriving and screening until the estimated binding of two or more candidates is adequate, wherein binding is adequate if the dissociation constant (K_{d}) of a synthesized candidate polypeptide from the selected target polypeptide is less than approximately 100 μM, whereby one or more candidate polypeptides are engineered to bind to one or more terminal peptide sequences of the selected target polypeptide.

128. A computer system for engineering one or more binding polypeptides from a selected precursor polypeptide, wherein the precursor polypeptide binds to one or more terminal peptide sequences of an initial target polypeptide, and wherein the binding polypeptides adequately bind to a selected target polypeptide, the system comprising:
a processor, and
a memory accessible to the processor, wherein the memory is configured with
(a) data for representing the precursor polypeptide, the initial target polypeptide, the selected target polypeptide, and further candidate polypeptides, and wherein
(i) the data for representing the selected target polypeptide consists essentially of data representing one or more of its terminal peptide sequences, wherein the one or more terminal peptide sequences of the selected target polypeptide comprise either an N-terminal, or a C-terminal peptide sequence, or both, with lengths of less than approximately 15 residues, and
(ii) the data representing the precursor polypeptide comprises data representing its amino acid sequence, its three-dimensional (3D) structure, and one or more of the amino acid sequences of the terminal peptide sequences of the initial target polypeptide bound by the precursor, wherein the one or more terminal peptide sequences of the initial target polypeptide comprise either an N-terminal or a C-terminal peptide sequence, or both with lengths of less than approximately 15 residues, and
(b) instructions for causing the processor, in dependence on the represented data, to perform the steps of
(i) rational engineering methods for deriving alternative candidate binding polypeptides by replacing one or more amino acid residues of a current candidate with new residues so that the alternative candidates are predicted to bind with one or more terminal peptide sequences of the selected target polypeptide,
(ii) rational binding-estimating methods comprising one or more computer-assisted molecular design (CAMD) methods for polypeptides for screening the alternative candidates for new candidates with improved estimated binding to terminal peptide sequences of the selected target, and
(iii) repeating, if necessary, the steps of rational engineering and estimating until the estimated binding of one or more candidates is adequate, wherein binding is adequate if the dissociation constant (K_{d}) of a synthesized candidate polypeptide from the selected target polypeptide is less than approximately 100 μM, whereby one or more candidate polypeptides are engineered to bind to one or more terminal peptide sequences of the selected target polypeptide.

129. The method of claim 128 wherein the terminal peptide sequences have lengths of less than approximately 10 residues.

130. A polypeptide for binding to a selected target polypeptide engineered according to the method of claim 123.

131. A method for assaying for one or more target polypeptides in a sample comprising:
contacting, in binding conditions, the sample with binding polypeptides, wherein one or more binding polypeptides are engineered by the method of claim 123 to bind to one or more of the target polypeptides, and
assaying for binding polypeptides bound to their respective target polypeptides, whereby the target polypeptides are assayed.

132. A polypeptide array comprising:
a substrate with at least one surface, and
a plurality of binding polypeptides regularly arranged on the surface,
wherein each binding polypeptide binds to one or more N-terminal or C-terminal peptide sequences, or both having lengths less than approximately 15 amino acids of each of a plurality of selected proteins expressed in an organism, and
wherein the plurality of selected expressed proteins comprises more than 500 different proteins and less than 50,000 different proteins.

133. A polypeptide array comprising:
a substrate with at least one surface, and
a plurality of binding polypeptides regularly arranged on the surface,
wherein each binding polypeptide binds to one or more N-terminal or C-terminal peptide sequences, or both, having lengths less than approximately 15 amino acids of each of a plurality of selected proteins expressed in an organism, and
wherein the plurality of selected expressed proteins comprises more than 5000 different proteins.
